(19) United States
(12) Patent Application Publication
(10) Pub. No.: US 2023/0416822 A1
(43) Pub. Date: Dec. 28, 2023

(54) DETECTION AND TREATMENT OF CONDITIONS CHARACTERIZED BY PERFUSION SHORTAGE

(71) Applicant: Falcon BioScience, LLC, Brooklyn, NY (US)

(72) Inventor: Henricus J. DUCKERS, Brooklyn, NY (US)

(21) Appl. No.: 17/926,306

(22) PCT Filed: May 19, 2021
(86) PCT No.: PCT/US2021/033254

§ 371 Date: Nov. 18, 2022

Related U.S. Application Data
(60) Provisional application No. 63/027,289, filed on May 19, 2020.

(51) Int. Cl. C12Q 1/6883

(52) U.S. Cl. CPC C12Q 1/6883; C12Q 2600/158

(57) ABSTRACT

Methods of detecting a condition comprising neoangiogenesis, ischemia, or both, or risk thereof in a subject. Methods of inhibiting, ameliorating, delaying the onset of, reducing the likelihood of, treating, or preventing a condition comprising perfusion shortage (such as neoangiogenesis, ischemia, or both) in a subject in need thereof are described. Kits are described.

### CROSS-REFERENCE

This application is a National Stage under 35 U.S.C. 371 of International Patent Application No. PCT/US2021/033254, filed May 19, 2021, which claims the benefit of U.S. Provisional Patent Application No. 63/027,289, filed May 19, 2020; the disclosures of all of which are incorporated herein by reference in their entirety.

### SEQUENCE LISTING

This application contains a Sequence Listing, which has been submitted in .txt file and is hereby incorporated by reference in its entirety. Said .txt file is named “Sequence Listing.txt”, was created on May 19, 2021, and is 1,804 bytes in size.

### FIELD

The present disclosure is related to detection, prevention, and treatment of conditions comprising perfusion shortage, such as conditions comprising neoangiogenesis, or ischemia, or both.

### BACKGROUND

Diseases comprising perfusion shortage can include neoangiogenesis, ischemia, or both. Ischemic diseases are diseases in which a lack of tissue perfusion (usually due to vascular disease) results in oxygen deprivation and pathology (for example, infarct, lack of function, or chest pain). Example ischemic disease include, but are not limited to, cardiovascular disease (e.g., angina pectoris, coronary artery disease. myocardial infarction, microvascular disease), cerebrovascular disease (e.g., TIA, stroke, microvascular disease) and peripheral artery disease (e.g., claudication). The prognosis and therapy responsiveness of other diseases are also dependent on tissue perfusion, ischemia, or both (including, but not limited to, oncological disease, diabetes mellitus, kidney disease, pulmonological disease, ophthalmological disease, gynecological disease, but also determines outcome and therapy response in for instance, but not limited to sepsis, inflammation, oncological disease and mechanical ventilation of patients).

### SUMMARY

In an aspect, the present disclosure describes methods for analyzing a biological sample, comprising (a) obtaining said biological sample from a subject; (b) wherein when said subject is a female subject, assaying expression levels of two or more genes from a female targeted set of genes to generate an output comprising said expression levels from (b) indicative of said female subject's having or risk of having a condition comprising perfusion shortage or neoangiogensis; and (c) wherein when said subject is a male subject, assaying expression levels of two or more genes from a male targeted set of genes to generate an output comprising said expression levels from (c) indicative of said male subject's having or risk of having a condition comprising perfusion shortage or neoangiogensis.

In some embodiments, the subject has or is at risk of having a condition comprising perfusion shortage. In some embodiments, the female targeted set of genes comprises a plurality of genes listed in Tables 1, 3, and 5. In some embodiments, the male targeted set of genes comprises a plurality of genes listed in Tables 2, 4, and 6. In some embodiments, the methods further comprise obtaining a range of control expression levels of said female targeted set of genes.

In some embodiments, the assaying comprises comparing said expression levels of (b) with said range of control expression levels of said female targeted set of genes. In some embodiments, the methods further comprise obtaining a range of control expression levels of said male targeted set of genes. In some embodiments, the assaying comprises comparing said expression levels of (c) with said range of control expression levels of said male targeted set of genes. In some embodiments, wherein when said expression levels from either (b) or (c) are outside said range of control expression levels, the subject has said condition comprising perfusion shortage or neoangiogenesis is at risk for having said condition.

In some embodiments, the biological sample comprises isolated hematopoietic endothelial precursor cells (EPCs), fractions thereof, or secretions thereof. In some embodiments, the hematopoietic endothelial precursor cells (EPCs) is Flk-1+. In some embodiments, the hematopoietic endothelial precursor cells do not express a cell surface marker, Flk-1. In some embodiments, the hematopoietic endothelial precursor cells are positive for makers selected from Flt1, Flt4, VEGFR1, CXCR4, CD31, CD34, CD105, CD133, Sca-1, and c-Kit. In some embodiments, the output from (b) and said output from (c) both have an accuracy greater than about 84%.

In another aspect, the present disclosure provides the methods for analyzing a biological sample obtained from a subject, comprising: (i) subjecting a plurality of hematopoietic endothelial precursor cells (EPCs), fractions of said plurality of hematopoietic EPCs, secretions of said plurality of hematopoietic EPCs, or any combination thereof isolated from said biological sample, to a gene expression analysis, wherein said gene expression analysis comprises assaying expression levels of two or more genes from a plurality of identified genes; and (ii) generating an output comprising said gene expression levels, wherein said gene expression levels are indicative of said subject's having or risk of having a condition comprising perfusion shortage at least in part based on said expression levels.

In some embodiments, the plurality of hematopoietic EPCs comprises a cell surface marker of Flk-1/KDR. In some embodiments, the plurality of hematopoietic EPCs does not comprise a cell surface marker of Flk-1/KDR. In some embodiments, the plurality of hematopoietic EPCs are positive for makers selected from Flt1, Flt4, VEGFR1, CXCR4, CD31, CD34, CD105, CD133, Sca-1, and c-Kit.

In another aspect, the present disclosure provides methods of analyzing a biological sample obtained from a subject, comprising: (i) subjecting a biological sample obtained from said subject to a gene expression analysis, wherein said gene expression analysis comprises assaying expression levels of two or more genes from a plurality of identified genes; and (ii) generating an output comprising said gene expression levels indicative that said subject has or is at risk of having a condition comprising perfusion shortage with an accuracy that is greater than 91%, wherein when said assaying comprises a method selected from the group consisting of cDNA chip array analysis, quantitative (RT) PCR, microarray analysis, multiplexing, Luminex analysis, nucleic acid sequencing, northern blot analysis, genomic high throughput sequencing, nanostring, massive parallel signature sequencing (MPSS), serial analysis of gene expression (SAGE), enzyme-linked immunosorbent assay (ELISA), protein sequencing, mass spectrometry (such as MALDI TOF, QTOF, or SELDI TOF), quantitative western blot analysis, and peptide barcoding.

In some embodiments, the biological sample comprises isolated hematopoietic endothelial precursor cells (EPCs), fractions, or secretions thereof. In some embodiments, the plurality of identified genes comprises a female targeted set of genes and a male targeted set of genes. In some embodiments, the female targeted set of genes comprises a plurality of genes listed in Tables 1, 3, and 5. In some embodiments, the male targeted set of genes comprises a plurality of genes listed in Tables 2, 4, and 6.

In some embodiments, the methods comprise obtaining a range of control expression levels of said female targeted set of genes or of said male targeted set of genes and comparing said gene expression levels from (i) of either claim 12 or claim 13 to said range of control expression levels. In some embodiments, the said expression levels from (i) of either claim 12 or claim 13 are outside said range of control expression levels, said subject has perfusion shortage or is at risk for having said condition comprising perfusion shortage.

In another aspect, the present disclosure provides methods of analyzing a biological sample, comprising: obtaining said biological sample from a subject that has or is at risk for having a condition comprising perfusion shortage, wherein said perfusion shortage is at an early stage and has not caused any detectable tissue damage; assaying expression levels of two or more genes from a plurality of identified genes to generate an output comprising said expression levels indicative of said subject's having or is at risk of having said condition comprising perfusion shortage.

In some embodiments, the perfusion shortage is neoangiogenesis. In some embodiments, the perfusion shortage is ischemic heart disease. In some embodiments, the subject is a female subject, further comprising assaying less than 60, 40, or 14 genes from said plurality of genes listed in Tables 1, 3, and 5. In some embodiments, the methods comprise assaying less than 14 genes from said plurality of genes listed in Tables 1, 3, and 5. In some embodiments, the subject is a male subject, further comprising assaying less than 40, 25, 15 genes from said plurality of genes listed in Tables 2, 4, and 6.

In some embodiments, the methods comprise assaying less than 15 genes from said plurality of genes listed in Tables 2, 4, and 6. In some embodiments, the output has an accuracy greater than about 99%. In some embodiments, the output has an accuracy greater than about 98%. In some embodiments, the output further comprises providing an assessment of a severity of said subject's perfusion shortage. In some embodiments, the output further comprises providing a prognosis report of said subject's perfusion shortage. In some embodiments, the output further comprises providing a response report of said subject upon, during, and/or after a medical intervention. In some embodiments, the output further comprises providing a monitoring report of said subject before or after receiving said medical intervention.

In some embodiments, the methods further comprise providing a treatment to said subject, wherein said subject has or is at risk of having a condition comprising perfusion shortage, at least in part based on said output. In some embodiments, the treatment comprises administering an anti- or pro-ischemic medical intervention, anti- or pro-neoangiogenesis medical intervention to the subject, invasive monitoring, advanced imaging, or any combinations thereof. In some embodiments, the medical intervention comprises a pharmacotherapy, or surgical or percutaneous procedure.

In some embodiments, the surgical or percutaneous procedure comprises a bypass surgery or a percutaneous coronary procedure. In some embodiments, the medical intervention comprises administering a pharmaceutical composition selected from the group consisting of beta-blockers, calcium channels antagonists, nitrates, aspirin, cholesterol-lowering compounds, angiotensin-converting enzyme (ACE) inhibitors, and ranolazine. In some embodiments, the medical intervention targets one or more genes listed in Tables 1-6. In some embodiments, the medical intervention further comprises modulating expression levels of one or more genes listed in Tables 1-6.

In some embodiments, at least based in part on response report, prognosis report, or both, the methods further comprise ceasing said medical intervention. In some embodiments, at least based in part on monitoring report, the methods further comprise employing advanced imaging comprising invasive angiography, advanced imaging, nuclear imaging, MSCT, MRI analysis, tissue biopsy, monitoring of mechanical ventilation, or a combination thereof. In some embodiments, the subject does not have any symptom. In some embodiments, the subject is at least diagnosed with one of ischemic cardiovascular disease, ischemic heart disease, coronary artery disease, cardiovascular congenital disease, stroke, transient ischemic attacks, microvascular disease, microvascular dementia, claudication intermittents, Burgers disease, Thromboangiitis obliterans, Buerger disease, lymphangiogenic disease, hypertrophic or dilating cardiomyopathy, heart failure, pulmonary and peripheral emboli, oncological disease (including solid tumors, and hematological and lymphogenic metastatic oncological disease), macro angiopathy, micro angiopathy, neo angiogenesis, diabetes mellitus, ophthalmic disease, nephrological disease, orthopedic disease, gynecological disease, Parkinson's disease, multiple sclerosis, inflammatory or auto-immune disease, erectile dysfunction, sports injury, Crohn's disease, colitis ulcerosa, pulmonary fibrosis, COPD, emphysema, and cystic fibrosis. In some embodiments, the subject has at least one symptom of chest pain, chest discomfort, shortness of breath, limited exercise tolerance, ventricular or atrial arrhythmia, profuse sweating, aphasia, loss of motor or sensory function, and vision disturbance. In some embodiments, the subject has at least one risk factor for having a condition comprising perfusion shortage, wherein said risk factor is selected from the group consisting of high blood pressure; glucose intolerance; family history of cardiovascular disease, hypercholesterolaemia, or dyslipidemia; aging; smoking; physical inactivity; obesity; previous cardiovascular disease; diagnosis of diabetes mellitus, kidney disease, peripheral artery disease, or metabolic syndrome; gender; high low density lipoprotein (LDL) level; high cholesterol level; and specific DNA polymorphism associated with higher cardiovascular risk.

In some embodiments, the hematopoietic EPCs are positive for makers selected from VEGFR2, CD309, Flt1, Flt4, KDR1, VEGFR1, CXCR4, CD31, CD34, CD105, CD133, Sca-1, and c-Kit. In some embodiments, the methods further comprise isolating said Flk1+ cells, fractions thereof, or secretions thereof from a biological sample of said subject. In some embodiments, the biological sample is selected from the group consisting of blood or blood fractions, saliva, urine, stool, cerebrospinal fluid, semen, vaginal secretions, sputum, sweat, breast milk, synovial fluid, mucus, tears, bile, gastric fluid, interstitial fluid, transudate, exudate, and tissue biopsy.

In some embodiments, the assaying further comprises detecting nucleic acid expression levels, splice variant expression levels, peptide expression levels, or any combination thereof corresponding to said plurality of genes listed in Tables 1-6.

In some embodiments, the methods further comprise detecting said nucleic acid expression levels or splice variant expression levels or both expression levels comprising employing cDNA chip array analysis, quantitative (RT) PCR, microarray analysis, multiplexing, Luminex analysis, nucleic acid sequencing, northern blot analysis, genomic high throughput sequencing, nanostring, massive parallel signature sequencing (MPSS), and serial analysis of gene expression (SAGE), or any combination thereof. In some embodiments, the methods comprise detecting peptide expression levels comprises employing enzyme-linked immunosorbent assay (ELISA), protein sequencing, mass spectrometry (such as MALDI TOF, QTOF, or SELDI TOF), quantitative western blot analysis, and peptide barcoding, or any combination thereof. In some embodiments, the methods further comprise transmitting said output onto an intranet or internet.

In some embodiments, the methods further comprise at least one of: identifying a previous episode of the condition in the subject when the expression levels of at least two genes from Tables 1-6 are outside of a range of control expression levels of said at least two genes from Tables 1-6; diagnosing the subject as having the condition when the expression levels of at least two genes from Tables 1-6 are outside of a range of control expression levels of said at least two genes from Tables 1-6; determining the subject to be at risk of the condition when the expression levels of at least two genes from Tables 1-6 are outside of a range of control expression levels of said at least two genes from Tables 1-6; prognosticating an outcome in the subject based on a presence of, or degree to which the expression levels of at least two genes from Tables 1-6 are outside of a range of control expression levels of said at least two genes from Tables 1-6; predicting a response of the subject to a medical intervention based on a presence of, or degree to which the expression levels of at least two genes from Tables 1-6 are outside of a range of control expression levels of said at least two genes from Tables 1-6; selecting a medical intervention for the subject, (for example such as, by way of a surrogate marker) based on a presence of, or degree to which the expression levels of at least two genes from Tables 1-6 are outside of a range of control expression levels of said at least two genes from Tables 1-6; selecting a dose and/or frequency of a medical intervention based on a presence of, or degree to which the expression levels of at least two genes from Tables 1-6 are outside of a range of control expression levels of said at least two genes from Tables 1-6; validating a candidate medical intervention based on an effect of the candidate medical intervention on a presence of, or degree to which the expression levels of at least two genes from Tables 1-6 are outside of a range of control expression levels of said at least two genes from Tables 1-6; measuring a response to a candidate medical intervention, wherein the measuring comprises the expression levels of at least two genes from Tables 1-6 are outside of a range of control expression levels of said at least two genes from Tables 1-6; monitoring a subject's response to a medical intervention based on the expression levels of at least two genes from Tables 1-6; or treating a subject with a nucleotide sequence listed in Tables 3 and 4 or a peptide sequence listed in Tables 3 and 4.

In another aspect, the present disclosure provides a kit comprising a plurality of probes that is configured to detect expression levels of two or more genes listed in Tables 1-6. In some embodiments, the plurality of probes is a nucleic acid probe configured to bind to a sequence of a gene listed in Tables 1-6. In some embodiments, the plurality of probes is an antibody configured to bind to a protein encoded by a gene listed in Tables 1-6.

In another aspect, the present disclosure provides methods of profiling gene expression for determining one or more genes as signature for a perfusion shortage risk, comprising: (a) assaying gene expression in a cell from a subject with a perfusion shortage risk, and a control set; (b) detecting a subset of genes expressed at an altered state in the cell of the subject compared to the control set; (c) using a machine learning algorithm for analysis of a combination of genes expressed at an altered state to determine the sufficient number of such genes to identify a gene expression signature for a perfusion shortage risk; (d) rectifying a possible accuracy score based on a follow-up monitoring period with the subject; (e) generating an output of one or more genes that is a signature for a perfusion shortage risk, such that the signature has a predictive accuracy of a perfusion shortage risk that exceeds 66%.

### BRIEF DESCRIPTION OF THE DRAWINGS

FIG. 1 shows a plot exemplifying percent accuracy (Accuracy %) scores to diagnose ischemic heart disease using various combinations of RNA probes in female subjects, with the indicated number of selected probes using different statistical methods as indicated. As used herein, “accuracy” has its ordinary and customary meaning as would be understood by one of ordinary skill in the art in view of this disclosure. It refers to the percentage of results that are neither false positives nor false negatives. Accuracy may be represented mathematically as 100%−((type 1 error)+(type 2 error)). Type 1 error may also be referred to as “alpha error.” Type 2 error may also be referred to as “beta error.” A combination of 8 to 14 RNA probes is able to predict ischemic heart disease in female patients with near 100% accuracy (or >99.95% accuracy). The X-axis depict the number of nucleotide RNA probe biomarkers in the diagnostic classifier. The Y-axis depict the accuracy of diagnosis for ischemic heart disease in a cohort of subjects with symptomatic chest pain (n=244). These subjects were referred by the general practitioner for further analysis of complaints suspect for ischemic heart disease (i.e. coronary artery disease; CAD disease) in the cardiology outpatient clinic. The correct diagnoses of these patients were prospectively and retrospectively verified at baseline using standard-of-care/best-of-care diagnostic analysis, and by clinical follow-up at 6 month follow-up The different prediction curves in the graph depict the different biostatistical analysis algorithms used in the analysis of female subjects: grey closed circles depict Diagonal Discriminant Analysis; black closed squares depict Fisher's Discriminant Analysis; black open squares depicts partial Least Squares w subsequent logistic regression analysis; black closed circles depict Partial Least Squares analysis w subsequent Random Forest analysis; black open circles depict Quadratic Discriminant Analysis.

FIG. 2 shows a plot exemplifying percent accuracy (Accuracy %) scores to diagnose ischemic heart disease using various combinations of exon biomarkers in female subjects with fitted curves for exons expression levels using different statistical methods according to some embodiments. A particular exon expression level is indicative of a gene expression level associated, which the gene comprises the particular exon region. The X-axis depict the number of nucleotide RNA Exon biomarkers in the diagnostic classifier. The Y-axis depict the accuracy of diagnosis for ischemic heart disease in a cohort of subjects with symptomatic chest pain (n=244). These subjects were referred by the general practitioner for further analysis of complaints suspect for ischemic heart disease (i.e. coronary artery disease; CAD disease) in the cardiology outpatient clinic. The correct diagnoses of these patients were prospectively and retrospectively verified at baseline using standard-of-care/best-of-care diagnostic analysis, and by clinical follow-up at 6-month follow-up. The different prediction curves in the graph depict the different biostatistical analysis algorithms used in the analysis of female subjects: grey closed circles depict Diagonal Discriminant Analysis; black closed squares depict Fisher's Discriminant Analysis; grey open circles depict Linear Discriminant Analysis; and black open squares depicts partial Least Squares w subsequent logistic regression analysis. A combination of 9 to 14 exon biomarkers is able to predict ischemic heart disease in female patients with about 98% accuracy.

FIG. 3 shows a plot exemplifying percent accuracy (Accuracy %) scores to diagnose ischemic heart disease using the expression levels of various combinations of genes (for instance as detected by nucleotide biomarkers) in female subjects. The X-axis depict the number of gene biomarkers in the diagnostic classifier. The Y-axis depict the accuracy of diagnosis for ischemic heart disease in a cohort of subjects with symptomatic chest pain (n=244). These subjects were referred by the general practitioner for further analysis of complaints suspect for ischemic heart disease (i.e. coronary artery disease; CAD disease) in the cardiology outpatient clinic. The correct diagnoses of these patients were prospectively and retrospectively verified at baseline using standard-of-care/best-of-care diagnostic analysis, and by clinical follow-up at 6-month follow-up. The different prediction curves in the graph depict the different biostatistical analysis algorithms used in the analysis of female subjects: grey closed circles depict Diagonal Discriminant Analysis; black closed squares depict Fisher's Discriminant Analysis; grey open circles depict Linear Discriminant Analysis; and black open squares depicts partial Least Squares w subsequent logistic regression analysis

FIG. 4 shows a plot exemplifying percent accuracy (Accuracy %) scores to diagnose ischemic heart disease using various combinations of RNA probes as nucleotide biomarkers in female subjects using Diagonal Discriminant Analysis versus Linear Discriminant Analysis. The X-axis depict the number of nucleotide RNA probe biomarkers in the diagnostic classifier. The Y-axis depict the accuracy of diagnosis for ischemic heart disease in a cohort of subjects with symptomatic chest pain (n=244). These subjects were referred by the general practitioner for further analysis of complaints suspect for ischemic heart disease (i.e. coronary artery disease; CAD disease) in the cardiology outpatient clinic. The correct diagnoses of these patients were prospectively and retrospectively verified at baseline using standard-of-care/best-of-care diagnostic analysis, and by clinical follow-up at 6-month follow-up. The different prediction curves in the graph depict the different biostatistical analysis algorithms used in the analysis of female subjects: black closed circles depict Diagonal Discriminant Analysis; and grey closed circles depict Linear Discriminant Analysis. A combination of 8 to 27 RNA probes as nucleotide biomarkers is able to predict ischemic heart disease in female patients with near 100% accuracy.

FIG. 5 shows a plot exemplifying percent accuracy (Accuracy %) scores to diagnose ischemic heart disease using various combinations of exon biomarkers in female subjects using Linear Discriminant Analysis. The X-axis depict the number of nucleotide RNA Exon biomarkers in the diagnostic classifier. The Y-axis depict the accuracy of diagnosis for ischemic heart disease in a cohort of subjects with symptomatic chest pain (n=244). These subjects were referred by the general practitioner for further analysis of complaints suspect for ischemic heart disease (i.e. coronary artery disease; CAD disease) in the cardiology outpatient clinic. The correct diagnoses of these patients were prospectively and retrospectively verified at baseline using standard-of-care/best-of-care diagnostic analysis, and by clinical follow-up at 6-month follow-up. The prediction curve in the graph depict the use of Linear Discriminant Analysis in the prediction of the disease allocation of the female subjects. A combination of 9 to 58 exon biomarkers as nucleotide biomarkers is able to predict ischemic heart disease in female patients with about 96-99% accuracy.

FIG. 6 shows a plot exemplifying percent accuracy (Accuracy %) to diagnose ischemic heart disease using various combinations of RNA probes as nucleotide biomarkers in male subjects. The X-axis depict the number of nucleotide RNA probe biomarkers in the diagnostic classifier. The Y-axis depict the accuracy of diagnosis for ischemic heart disease in a cohort of subjects with symptomatic chest pain (n=244). These subjects were referred by the general practitioner for further analysis of complaints suspect for ischemic heart disease (i.e. coronary artery disease; CAD disease) in the cardiology outpatient clinic. The correct diagnoses of these patients were prospectively and retrospectively verified at baseline using standard-of-care/best-of-care diagnostic analysis, and by clinical follow-up at 6-month follow-up. The different prediction curves in the graph depict the different biostatistical analysis algorithms used in the analysis in male subjects: grey closed circles depict Diagonal Discriminant Analysis; black closed squares depict Fisher's Discriminant Analysis; and black open circles depict Quadratic Discriminant Analysis. A combination of 14 to 40 RNA probes is able to predict ischemic heart disease in male patients with about 98% accuracy.

FIG. 7 shows a plot exemplifying percent accuracy (Accuracy %) to diagnose ischemic heart disease using the expression of various combinations of genes (for instance as detected by nucleotide biomarkers) in male subjects. The X-axis depict the number of genetic biomarkers in the diagnostic classifier. The Y-axis depict the accuracy of diagnosis for ischemic heart disease in a cohort of subjects with symptomatic chest pain (n=244). These subjects were referred by the general practitioner for further analysis of complaints suspect for ischemic heart disease (i.e. coronary artery disease; CAD disease) in the cardiology outpatient clinic. The correct diagnoses of these patients were prospectively and retrospectively verified at baseline using standard-of-care/best-of-care diagnostic analysis, and by clinical follow-up at 6-month follow-up. The different prediction curves in the graph depict the different biostatistical analysis algorithms used in the analysis of male subjects: grey closed circles depict Diagonal Discriminant Analysis; black closed squares depict Fisher's Discriminant Analysis; grey open circles depict Linear Discriminant Analysis; black open squares depicts partial Least Squares w subsequent logistic regression analysis; black closed circles depict Partial Least Squares analysis w subsequent Random Forest analysis; black open circles depict Quadratic Discriminant Analysis; and black diamonds depict Random Forrest Analysis. A combination of 14 to 22 nucleotide biomarkers (to indicate corresponding specific genes expression levels) is able to predict ischemic heart disease in male patients with about 93-96% accuracy.

FIG. 8 provides an illustrative framework for a clinical study for ischemic biomarker diagnostic methods.

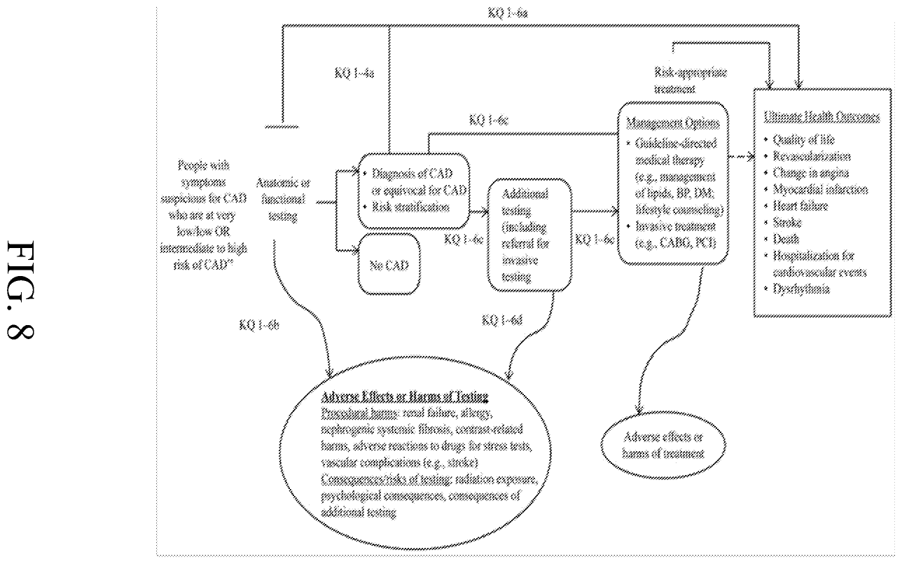

FIG. 9 provides a thorough summary of sensitivity and specificity results of invasive tests currently used to diagnosis coronary artery disease.

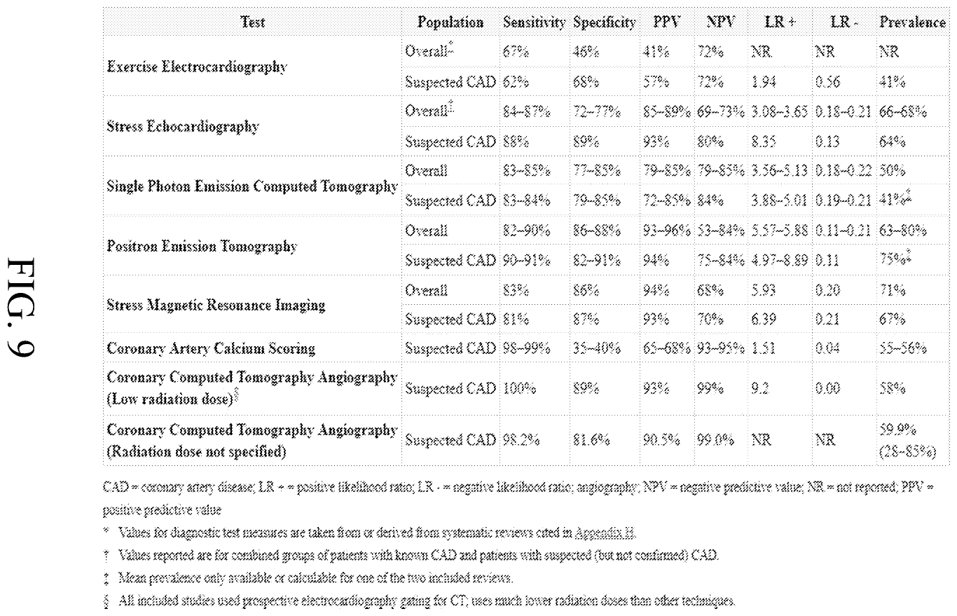

FIG. 10 provides a study assessment schedule for the clinical study outlined in FIG. 11.

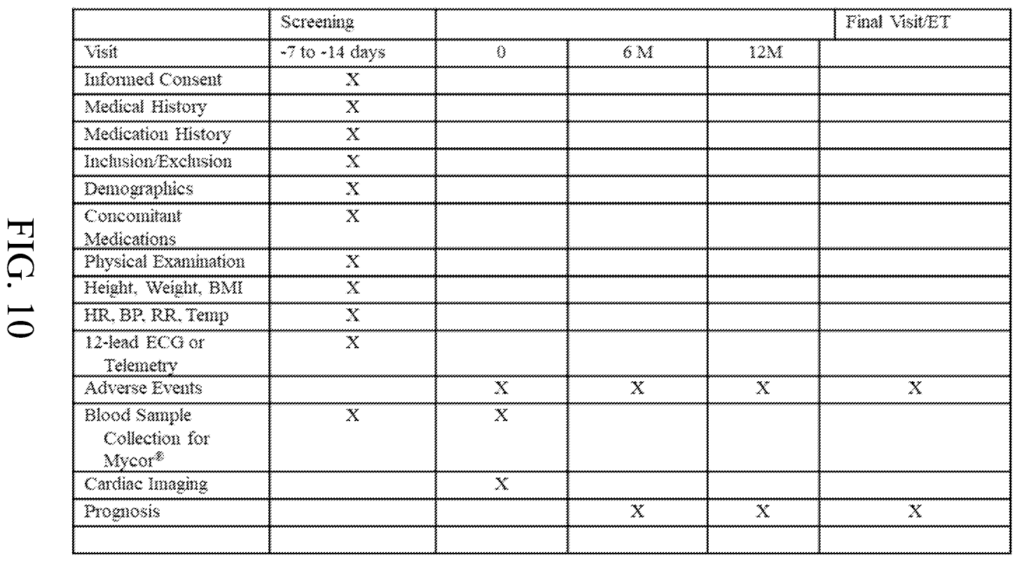

### DETAILED DESCRIPTION

Ischemia in cardiovascular patients is typically caused by a coronary artery stenosis and/or occlusion (due to progressive coronary atherosclerosis), leading to a local low oxygen (ischemic) condition in the heart, and may eventually result in myocardial damage and irreversible loss of cardiac contractile function. The narrowing of blood vessels is usually caused by atherosclerosis (fat deposition/inflammation in the vessel wall). However, the presence of atherosclerosis per se does not mean there is oxygen deprivation. Although ischemia accounts for the onset of heart disease, early ischemic conditions can be quiescent or transient, and therefore may escape detection by conventional diagnostic methods. Further, some available biomarkers are correlated with a greater chance to develop atherosclerosis, which may or may not lead to ischemia.

Described herein are methods, compositions, kits, and uses for detecting a condition or a likelihood of developing such condition comprising perfusion shortage, such as a condition comprising neoangiogenesis or ischemia, or a risk of such a condition. Without being limited by theory, it is contemplated that ischemia or neoangiogenesis can form the basis of the pathogenesis of some diseases (like ischemic heart or cerebrovascular disease) or determine the prognosis, therapy responsiveness or risk for other diseases). The present disclosure also describes a gender-specific or sex-based approach in detecting the condition or a likelihood of developing such condition comprising perfusion shortage or a risk thereof. In addition, the present disclosure presents methods for detecting conditions comprising ischemia or a risk thereof in a subject when the subject may not have developed any detectable tissue damages due to ischemic conditions.

Further, the present disclosure describes methods for identifying and measuring blood markers that are activated due to an oxygen stress response, which may be independent of atherosclerosis. In some aspects, hematopoietic endothelial precursor cells (EPCs) positive for Flk1+ (or its human orthologue, VEGFR2 or KDR) (for convenience, the cells may be referred to herein as “Flk1+ cells,” or “Flk1-positive cells”), and optionally at least one of Flt1, Flt4, KDR1, VEGFR1, VEGFR2, CD309, CXCR4, CD31, CD34, CD105, CD133, Sca-1, and c-Kit can exhibit different expression profiles of genes in subjects that are at risk for conditions comprising perfusion shortage (for example conditions comprising neoangiogenesis and/or ischemia). In some cases, the expression profiles of genes can be generated by measuring or detecting the genes' corresponding transcripts, splice variants, exon regions, or peptides. In some embodiments, the genes of interest are listed in Tables 1-6 attached as appendix herein.

Moreover, the method, composition, use, or kit (e.g., protocol, biomarkers, reference values, and/or algorithm) can be used for the identification of current and past ischemic events (irrespective of its location), or can be used for the selection of patients for more advanced diagnostic workup or initiation/cessation or modulation of specific therapy, for example to measure a response to the therapy (such as a “gate keeper” or “companion diagnostic” use, rather than a stand-alone diagnostic). The methods, compositions, uses, or kits of some embodiments are therefore able to (with sufficient accuracy) to exclude diseases comprising perfusion shortage (such as neoangiogenesis and/or ischemia) in subjects, as well as identify such diseases.

### Gender-Specific Approach in Detecting a Condition Comprising Perfusion Shortage or a Risk Thereof

Researches have shown that there are sex-related differences between women and men. These sex-related differences may include varying risk factor profiles, accuracy of diagnostic testing, clinical presentations, treatment practices, and outcomes. For example, women have been shown to present cardiovascular symptoms late, and some of these symptoms may not recognized by health care professionals. In oncology, sex-related differences also include incidence, outcome, and response to therapies after adjusting of known epidemic risk factors. Specifically, large differences in mutation density and frequency of mutation of specific oncogenes have been shown, such as 0-catenin and BAP1.

The present disclosure discloses a gender specific approach in detecting a condition comprising perfusion shortage or a risk thereof. In some cases, the methods for analyzing a biological sample comprises obtain the biological sample from the subject. When the subject is a female, a female targeted set of genes is identified and utilized. Further, a set of nucleotide sequences and peptide sequences encoded therefrom as disclosed in Table 3 in female subjects is also identified and utilized. Expression levels of at least two genes (or nucleotide or protein biomarkers) from the female targeted set of genes are measured or detected. Based on the signals detected or measured from the at least two genes (or nucleotide or protein biomarkers) from the female targeted set of genes, an output indicative of the female subject's having or at risk of having the condition comprising perfusion shortage may be generated and reported to a health care professional. In some cases, the methods further comprise obtaining a range of control expression levels of said female targeted set of genes (or nucleotide or protein biomarkers). Tables 1, 3, and 5 attached herein as appendix comprise the female targeted set of genes (including nucleotide sequences that can be used to detect gene expression levels or protein biomarkers that can be used to detect protein expression levels). Table 3 also lists a first plurality of nucleotide sequences that are identified to detect the expression levels of corresponding genes. The listed first plurality of nucleotide sequences are not meant to be limiting as other regions of genes can also be used to detect expression levels of genes. Further, Table 3 lists a send plurality of nucleotide sequences that can hybridize to the first plurality of nucleotide sequences. Additionally, Table 3 lists a third plurality of peptide sequences that are encoded by the first plurality of nucleotide sequences. Peptide sequences (e.g., antibodies (or protein ligands)) that may hybridize to the third plurality of peptide sequences may be used to detect expression levels of the corresponding genes (or protein expression levels). In some cases, the female targeted set of genes does not overlap with the male targeted set of genes. Moreover, Table 5 comprises a set of exon regions of genes in female subjects.

Further, when the subject is a male, a male targeted set of genes is identified and utilized. Further, a set of nucleotide sequences and peptide sequences encoded therefrom as disclosed in Table 4 in female subjects is also identified and utilized. Expression levels of at least two genes (or nucleotide or protein biomarkers) from the male targeted set of genes (or nucleotide or protein biomarkers) are measured or detected. Based on the signals detected or measured from the at least two genes (or nucleotide or protein biomarkers) from the male targeted set of genes, an output indicative of the male subject's having or at risk of having the condition comprising perfusion shortage may be generated and reported to a health care professional. In some cases, the methods further comprise obtaining a range of control expression levels of said male targeted set of genes (or nucleotide or protein biomarkers). Tables 2, 4, and 6 attached herein as appendix comprise the male targeted set of genes (including nucleotide sequences that can be used to identify genes and peptide sequences encoded therefrom). Table 4 also lists a first plurality of nucleotide sequences that are identified to detect the expression levels of corresponding genes. The listed first plurality of nucleotide sequences are not meant to be limiting as other regions of genes can also be used to detect expression levels of genes. Further, Table 4 lists a send plurality of nucleotide sequences that can hybridize to the first plurality of nucleotide sequences. Additionally, Table 4 lists a third plurality of peptide sequences that are encoded by the first plurality of nucleotide sequences. Peptide sequences (e.g., antibodies (or protein ligands)) that may hybridize to the third plurality of peptide sequences may be used to detect expression levels of the corresponding genes or proteins. Moreover, Table 6 comprises a set of exon regions of genes in male subjects.

In some cases, pilot data in murine and swine models of ischemia had shown that certain genes were indeed upregulated upon an ischemic myocardial event. Moreover, upregulated expression levels of other specific marker genes as disclosed herein has also been detected in circulating mononuclear cells in the blood of patients with myocardial ischemic events by use of qPCR analysis or any other suitable methods. The differentially expression levels of genes described herein (which have been observed to be differentially expressed in Flk1+ cells or fractions or secretions thereof in subjects suffering from hypoxemia), can comprise any nucleotide sequence, transcript, splice variant, and/or peptide identified by any of SEQ ID NOs: 1-5103, Genes Number 1-9280 (from Tables 1, 2, 5 & 6), or a peptide sequence encoded therefrom. Differential expression levels of these gene(s) as described herein can be indicative of a presence, risk, or severity of a condition comprising perfusion shortage (such as a condition comprising neoangiogenesis or ischemia). Without being limited by theory, the differentially expressed genes identified during hypoxemia as described herein (e.g., any nucleotide sequence, transcript, splice variant, and/or peptide identified by any of SEQ ID NOs: 1-5103, Genes Number 1-9280, or a peptide sequence encoded therefrom) may, in some embodiments, also represent a therapeutic target or lead to promote or regulate (the reactive hypoxemic) neoangiogenesis response and vascular modelling in disease. By enhancing, stabilizing, inhibiting, or ameliorating the gene expression levels of potential therapeutic target(s) or lead(s) or interfering with protein deliveries of potential therapeutic target(s) or lead(s), neoangiogenesis may be inhibited to treat diseases involving neoangiogenesis, for instance, not meant to be limiting, ophthalmic/retinal neoangiogenesis conditions. Also, tumor angiogenesis may be inhibited to slow down the growth a solid tumor and deteriorate tumor growth environment.

Further, in some case, a female targeted set of genes is listed in Tables 1, 3, and 5. In some cases, a male targeted set of genes is listed in Tables 2, 4, and 6. A control range of the expression levels of genes listed in Tables 1, 3, and 5 and listed in Tables 2, 4, and 6 are obtained from healthy subjects that do not have oxygen stress response due to ischemic conditions. In certain embodiments, when expression levels of the two or more genes from the female targeted genes are measure or detected outside of the control ranges in a female subject, an output or a report may be generated to indicate that the female subject might have or at risk of having a condition comprising perfusion shortage. Moreover, in certain embodiments, when expression levels of the two or more genes from the male targeted genes are measure or detected outside of the control ranges in a male subject, an output or a report may be generated to indicate that the male subject might have or at risk of having a condition comprising perfusion shortage.

In some embodiments, a method for detecting a condition comprising perfusion shortage (such as a condition comprising neoangiogenesis or ischemia), or risk thereof in a subject is described. The method can comprise detecting, in a biological sample of the subject, expression levels of at least two specified different genes (or nucleotide sequences or peptide biomarkers) listed in Tables 1, 3, and 5 for female subjects and Tables 2, 4, and 6 for male subjects. The method may further comprise detecting expression levels of exon regions as disclosed in Table 5 for female subjects and in Table 6 for male subjects. As disclosed herein, nucleotide and peptide sequences encoded therefrom listed in Tables 3 and 4 maybe used to measure expression levels of corresponding genes. The method can comprise receiving a set of control ranges of expression levels of the specified different gene products (as described herein, the control ranges can be determined experimentally, or received as a set of stored values, for example electronically or optically stored values). The method can comprise detecting the condition comprising neoangiogenesis or ischemia or a risk thereof when the expression levels of the specified different genes are outside of the control ranges. By way of example, the expression level of a DNA, RNA, splice variant, peptide, or a combination thereof of a gene from either a female or male targeted set of genes can be measured or detected. The method may comprise determining the copy number, level of expression, or level of activity of the at least two specified different genes (or nucleotide sequences or peptide biomarkers) in a biological sample from the subject. In some embodiments, the method comprises comparing the copy number, level of expression, or level of activity of the at least two specified different gene products with control ranges of copy number, level of expression, or level of activity, in which a significantly modulated copy number, level of expression, or level of activity of the target, relative to the control ranges, is an indication of neoangiogenesis or ischemia, or risk thereof in the subject. By way of example, expression levels of the genes can be determined to be outside of the control range using a statistical method selected from the group consisting of Fisher Discriminant analysis (FDA), Diagonal linear discriminant analysis (DLDA), Linear discriminant analysis (LDA), linear discriminant analysis based on partial least-squares (PLSLDA), partial least squares random Forest (PLSRF), orthogonal discriminant analysis (QDA), and Random Forest (RF), or a combination of any two of the listed methods. In some embodiments, the method is an in vitro method.

In some cases, the at least two specified different gene products correspond to two different genes selected from Tables 1-6. In some cases, the at least two specified different gene products comprise at least two different sequences in SEQ ID NOs: 1-5103, Genes Number 1-9280, or a peptide sequence encoded therefrom. The at least two different sequences may be any combination of different sequences or gene products among SEQ ID NOs: 1-5103, Genes Number 1-9280, or a peptide sequence encoded therefrom. For example, the method, composition, kit, or use of some embodiments can comprise measuring or detecting the expression levels of at least 2, 3, 4, 5, 6, 7, 8, 9, 10, 11, 12, 13, 14, 16, 17, 18, 19, 20, 21, 22, 23, 24, 25, 26, 27, 28 29, 30, 31, 32, 33, 34, 35, 36, 37, 38, 39, 40, 50, or genes from Tables 1-6, including ranges between any two of the listed values, for example, 2-14, 2-2-16, 2-17, 2-18, 2-19, 2-20, 2-40, 20-50, 20-60, 4-14, 4-15, 4-20, 4-25, 4-30, 4-35, 4-40, 4-50, 4-5-10, 5-14, 5-15, 5-16, 5-17, 5-18, 5-19, 5-20, 5-40, 5-50, 5-60, 6-14, 6-15, 6-20, 6-25, 6-30, 6-35, 6-40, 6-50, 6-60, 8-14, 8-15, 8-20, 8-25, 8-30, 8-35, 8-40, 8-50, 8-60, 9-10, 9-14, 9-15, 9-16, 9-20, 9-9-50, 9-60, 10-14, 10-15, 10-20, 10-25, 10-30, 10-35, 10-40, 10-50, or 10-60 genes from Tables 1-6. In some embodiments, for example, if the subject is male, the methods disclosed herein comprise assaying no more than 40 different genes, for example no more than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 6, 5, 4, 3 or 2 different genes (or nucleotide or protein biomarkers) from Tables 2, 4, and 6, for example 2-14, 2-15, 2-16, 2-17, 2-18, 2-19, 2-20, 2-40, 20-50, 20-60, 4-14, 4-15, 4-20, 4-25, 4-30, 4-35, 4-40, 5-10, 5-14, 5-16, 5-17, 5-18, 5-19, 5-20, 5-40, 5-50, 5-60, 6-14, 6-15, 6-20, 6-25, 6-30, 6-35, 6-40, 8-14, 8-8-20, 8-25, 8-30, 8-35, 8-40, 8-50, 8-60, 9-10, 9-14, 9-15, 9-16, 9-20, 9-40, 10-14, 10-15, 10-20, 10-30, 10-35, or 10-40 different genes (or nucleotide or protein biomarkers). In some embodiments, for example, if the subject is female, the methods disclosed herein comprise assaying no more than 40 different genes, for example no more than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 6, 5, 4, 3 or 2 different genes (or nucleotide or protein biomarkers) from Tables 1, 3, and 5, for example, 2-14, 2-15, 2-16, 2-17, 2-18, 2-19, 2-20, 2-40, 20-20-60, 4-14, 4-15, 4-20, 4-25, 4-30, 4-35, 4-40, 4-50, 4-60, 5-10, 5-14, 5-15, 5-16, 5-17, 5-18, 5-19, 5-20, 5-40, 5-50, 5-60, 6-14, 6-15, 6-20, 6-25, 6-30, 6-35, 6-40, 6-50, 6-60, 8-14, 8-15, 8-20, 8-25, 8-30, 8-35, 8-40, 8-50, 8-60, 9-10, 9-14, 9-15, 9-16, 9-20, 9-40, 9-50, 9-60, 10-14, 10-15, 10-20, 10-10-30, 10-35, 10-40, 10-50, or 10-60 different genes (or nucleotide or protein biomarkers).

In some embodiments, when the expression levels of the at least two different gene products are outside of the control ranges, the condition comprising perfusion shortage, or a risk thereof is detected. For example, if the expression levels of the at least two different genes (or nucleotide or protein biomarkers) exceed the control range, the condition or risk thereof may be detected. In some embodiments, the expression levels of the at least two different genes (or nucleotide or protein biomarkers) are outside of the control ranges, and the condition or a risk thereof is detected.

In the method of some embodiments, when the expression levels of about 4 to about 40, about 4 to about 60, about 6 to about 40, about 6 to about 60, about 8 to about 40, about 8 to about 60, about to about 40, or about 10 to about 60 specified different genes are outside of the control ranges, and the condition comprising perfusion shortage or a risk thereof is detected. In the method of some embodiments, when the expression levels of about 4 to about 30 specified different genes are outside of the control ranges, and the condition or a risk thereof is detected. In the method of some embodiments, when the expression levels of about 4 to about 20 specified different gene products are outside of the control ranges, and the condition or a risk thereof is detected. In the method of some embodiments, when the expression levels of about 6 to about 40 specified different gene products are outside of the control ranges, and the condition or a risk thereof is detected. In the method of some embodiments, when the expression levels of about 6 to about 30 specified different gene products are outside of the control ranges, and the condition or a risk thereof is detected. In the method of some embodiments, when the expression levels of about 6 to about 20 specified different gene products are outside of the control ranges, and the condition or a risk thereof is detected. In the method of some embodiments, when the expression levels of about 2 to about 30 specified different gene products are outside of the control ranges, and the condition or a risk thereof is detected. In the method of some embodiments, when the expression levels of about 5 to about 10 specified different gene products are outside of the control ranges, and the condition or a risk thereof is detected. In the method of some embodiments, when the expression levels of about 8 to about 14 specified different gene products are outside of the control ranges, and the condition or a risk thereof is detected. In the method of some embodiments, when the expression levels of about 10 to about 15 specified different gene products are outside of the control ranges, and the condition or a risk thereof is detected. In the method of some embodiments, the expression levels of fewer than 15 specified different genes are outside of the control ranges, and the condition or a risk thereof is detected. In the method of some embodiments, when the expression levels of about 15 to about 20 specified different gene products are outside of the control ranges, and the condition or a risk thereof is detected.

In some embodiments, when the subject is female, the method comprises detecting the condition comprising perfusion shortage or a risk thereof in the subject when the expression levels of at least two specified different genes (or nucleotide or protein biomarkers) that each comprise a sequence from SEQ ID NOs: 1-5103, Genes Number 1-9280, or a peptide sequence encoded therefrom are outside of the control ranges, for example 2-60 or 2-40 specified different genes, including subranges within the listed range, for example, about 2-14, 2-15, 2-16, 2-17, 2-18, 2-19, 2-20, 2-40, 20-20-60, 4-14, 4-15, 4-20, 4-25, 4-30, 4-35, 4-40, 4-50, 4-60, 5-10, 5-14, 5-15, 5-16, 5-17, 5-18, 5-19, 5-20, 5-40, 5-50, 5-60, 6-14, 6-15, 6-20, 6-25, 6-30, 6-35, 6-40, 6-50, 6-60, 8-14, 8-15, 8-20, 8-25, 8-30, 8-35, 8-40, 8-50, 8-60, 9-10, 9-14, 9-15, 9-16, 9-20, 9-40, 9-50, 9-60, 10-14, 10-15, 10-20, 10-10-30, 10-35, 10-40, 10-50, or 10-60 specified different genes that each comprise a sequence from Tables 1, 3, and 5 (or peptide sequence encoded therefrom). Other suitable nucleotide sequences or probes that can detect and measure the expression levels of genes listed in Tables 1, 3, and 5 may also be used to detect the condition or the likelihood of developing such condition comprising perfusion shortage in female subjects. Moreover, other suitable peptide sequences that can detect and measure the expression levels of genes listed in Tables 1, 3, and 5 may also be used to detect the condition or the likelihood of developing such condition comprising perfusion shortage in female subjects. When the subject is male, the method comprises detecting the condition comprising perfusion shortage or a risk thereof in the subject when the expression levels of at least two specified different genes that each comprise a sequence from SEQ ID NOs: 1-5103, Genes Number 1-9280, or a peptide sequence encoded therefrom are outside of the control ranges, for example 2-60 or 2-40 specified different genes, including subranges within the listed range, for example, about 2-14, 2-15, 2-16, 2-17, 2-18, 2-19, 2-2-40, 4-14, 4-15, 4-20, 4-25, 4-30, 4-35, 4-40, 5-10, 5-14, 5-15, 5-16, 5-17, 5-18, 5-19, 5-20, 5-40, 6-14, 6-15, 6-20, 6-25, 6-30, 6-35, 6-40, 8-14, 8-15, 8-20, 8-25, 8-30, 8-35, 8-40, 9-10, 9-14, 9-15, 9-16, 9-20, 9-40, 10-14, 10-15, 10-20, 10-25, 10-30, 10-35, or 10-40 specified different genes that each comprise a sequence from Tables 2, 4, and 6 (or peptide sequence encoded therefrom). Other suitable nucleotide sequences or probes that can detect and measure the expression levels of genes listed in Tables 2, 4, and 6 may also be used to detect the condition or the likelihood of developing such condition comprising perfusion shortage in male subjects. Moreover, other suitable peptide sequences that can detect and measure the expression levels of genes listed in Tables 2, 4, and 6 may also be used to detect the condition or the likelihood of developing such condition comprising perfusion shortage in male subjects. In some embodiments, the same numerical of genes is detected for male and female subjects.

In some embodiments, when the subject is female, the method comprises detecting the condition comprising perfusion shortage or a risk thereof in the subject when the expression levels of at least two specified different genes (or nucleotide or protein biomarkers) from Tales 1, 3, and 5 are outside of the control ranges, for example 2-60 specified different genes, including subranges within the listed range, for example, 2-14, 2-15, 2-16, 2-17, 2-18, 2-19, 2-20, 2-40, 20-50, 20-60, 4-14, 4-15, 4-20, 4-25, 4-30, 4-35, 4-40, 4-50, 4-60, 5-10, 5-14, 5-15, 5-16, 5-17, 5-18, 5-19, 5-20, 5-40, 5-50, 5-6-14, 6-15, 6-20, 6-25, 6-30, 6-35, 6-40, 6-50, 6-60, 8-14, 8-15, 8-20, 8-25, 8-30, 8-35, 8-40, 8-50, 8-60, 9-10, 9-14, 9-15, 9-16, 9-20, 9-40, 9-50, 9-60, 10-14, 10-15, 10-20, 10-25, 10-30, 10-35, 10-40, or 10-60 specified different genes (or nucleotide or protein biomarkers).

In some embodiments, when the subject is male, the method comprises detecting the condition or a risk thereof in the subject when the expression levels of at least two specified different genes (or nucleotide or protein biomarkers) from Tables 2, 4, and 6 are outside of the control ranges, for example at least 2 and less than 15, 14, 13, 12, 11, or 10 specified different genes (or nucleotide or protein biomarkers).

In some embodiments, when the subject is female, the method comprises detecting, from the biological sample, expression levels of only 2-40 specified different genes from Tables 1, 3, and 5, including subranges within the listed range, for example, about 2-14, 2-15, 2-16, 2-17, 2-18, 2-19, 2-2-40, 4-14, 4-15, 4-20, 4-25, 4-30, 4-35, 4-40, 5-10, 5-14, 5-15, 5-16, 5-17, 5-18, 5-19, 5-20, 5-40, 6-14, 6-15, 6-20, 6-25, 6-30, 6-35, 6-40, 8-14, 8-15, 8-20, 8-25, 8-30, 8-35, 8-40, 9-10, 9-14, 9-15, 9-16, 9-20, 9-40, 10-14, 10-15, 10-20, 10-25, 10-30, 10-35, or 10-40 specified different genes. When the subject is male, the method comprises detecting, from the biological sample, expression levels of only 2-40 specified different genes (or nucleotide or protein biomarkers) from Tables 2, 4, and 6, for example, only 2-14, 2-15, 2-16, 2-17, 2-18, 2-19, 2-20, 2-40, 4-14, 4-15, 4-20, 4-25, 4-30, 4-35, 4-40, 5-14, 5-15, 5-16, 5-17, 5-18, 5-19, 5-20, 5-40, 6-14, 6-15, 6-20, 6-25, 6-30, 6-35, 6-40, 8-14, 8-8-20, 8-25, 8-30, 8-35, 8-40, 9-10, 9-14, 9-15, 9-16, 9-20, 9-40, 10-14, 10-15, 10-20, 10-25, 10-30, or 10-40 specified different genes (or nucleotide or protein biomarkers). In some embodiments, the same numerical of gene products is detected for male and female subjects.

In some embodiments, when the subject is female, the method comprises detecting, from the sample, expression levels of only 2-14, 2-15, 2-16, 2-17, 2-18, 2-19, 2-20, 2-40, 4-14, 4-15, 4-20, 4-25, 4-30, 4-35, 4-40, 5-10, 5-14, 5-15, 5-16, 5-17, 5-18, 5-19, 5-20, 5-40, 6-14, 6-15, 6-20, 6-25, 6-30, 6-6-40, 8-14, 8-15, 8-20, 8-25, 8-30, 8-35, 8-40, 9-10, 9-14, 9-15, 9-16, 9-20, 9-40, 10-14, 10-15, 10-10-25, 10-30, 10-35, or 10-40 specified different genes (or nucleotide or protein biomarkers) from Tables 1, 3, and 5. In some embodiments, the same numerical of gene products is detected for male and female subjects. In some embodiments, when the subject is male, the method comprises detecting, from the sample, expression levels of only 2-14, 2-15, 2-16, 2-17, 2-18, 2-19, 2-20, 2-40, 4-14, 4-15, 4-20, 4-25, 4-30, 4-35, 4-40, 5-10, 5-14, 5-15, 5-16, 5-17, 5-18, 5-19, 5-20, 5-40, 6-14, 6-15, 6-20, 6-25, 6-6-35, 6-40, 8-14, 8-15, 8-20, 8-25, 8-30, 8-35, 8-40, 9-10, 9-14, 9-15, 9-16, 9-20, 9-40, 10-14, 10-10-20, 10-25, 10-30, 10-35, or 10-40 specified different genes (or nucleotide or protein biomarkers) from Tables 2, 4, and 6.

In some embodiments, receiving a set of control ranges of expression levels of the specified different gene products comprises receiving a set of electronically or optically stored values. For example, control ranges may be obtained from a reference, such as an electronic database in which control ranges are stored.

In some embodiments, receiving a set of control ranges of expression levels of the specified different gene products comprises detecting expression levels of the specified different gene products of isolated hematopoietic endothelial precursor cells (EPCs), which includes EPCs expressing surface marker Flk1, or EPCs expression other surface markers, such as Flt1, Flt4, CD31, CD34 and more, of a control individual who does not have the condition and does not have a risk thereof.

### Methods for Detecting a Condition Comprising Perfusion Shortage Using Hematopoietic Endothelial Precursor Cells (EPCs) Cells, Fractions or Secretions Thereof

Blood-based diagnostics are challenged by a high abundances of non-biomarker species, such as albumin, globulins, and blood group glycans, and relatively low abundances of biomolecules useful for disease and risk assessment. Many blood biomarkers correlate with the current health status of a patient, and provide minimal insight into the patient's propensity for developing a disease or condition. Furthermore, as blood biomolecules are derived from disparate cell and tissue types, direct blood analysis can be blind to risk factors for localized conditions.

The present disclosure provides a range of strategies which circumvent these challenges and enable biological state and disease predisposition diagnoses patient blood samples. While many blood biomarkers correlate weakly with perfusion shortage and related factors (e.g., neoangiogenesis or ischemia), the expression patterns of hematopoietic lineage endothelial precursor cells (EPCs), and in particular cases Flk1+EPCs, can be highly responsive to perfusion shortage risk and development. In some aspects, the present disclosure presents methods for analyzing a biological sample obtained from a subject. The methods comprise subjecting a plurality of hematopoietic lineage endothelial precursor cells (EPCs), fractions of said plurality of hematopoietic lineage EPCs, secretions of said plurality of hematopoietic lineage EPCs, or any combination thereof isolated from said biological sample, to a gene (nucleotide or protein) expression analysis, wherein said gene (nucleotide or protein) expression analysis comprises assaying expression levels of two or more genes (nucleotides or proteins) from a plurality of identified genes (nucleotides or proteins); and generating an output comprising said gene (nucleotide or protein) expression levels, wherein said gene (nucleotide or protein) expression levels are indicative of said subject's having or risk of having a condition comprising perfusion shortage at least in part based on said expression levels. Hematopoietic lineage EPCs or other vascular precursor cells that have the same characteristics are involved in the neoangiogenesis response. For example, hematopoietic lineage EPCs respond and react to low oxygen conditions such as low perfusion, hypoxemia, or ischemia, and play a role in new blood vessel formation at the site of low oxygen condition in order to correct such condition.

In some cases, hematopoietic EPCs may be Flk1+, meaning, the EPCs express FlK1 receptors on cell surface (maybe referred as Flk1+ cells). Flk1 may be referred to as “VEGFR2,”, “CD309”, “Kdr” and “Kdr1,” and these terms may be used interchangeably herein. In a biological sample of a certain organism, the relevant Flk1 orthologue for that organism is detected. For example, in a human biological sample (or for a human subject), “Flk1” or “Kdr1” or “VEGFR2” is understood to refer to the Homo sapiens orthologue of this gene. As used herein, “Flk1+ cell” or fractions or secretions thereof” (including variations of these root terms) has its ordinary and customary meaning as would be understood by one of ordinary skill in the art in view of this disclosure. The Flk1+ phenotype may identify endothelial precursor cells, and the shorthand “Flk1+ cell” refers to cells that are positive for Flk1. The Flk1+ cells may be further positive for markers selected from Flt1 (also referred to as VEGFR1), Flt4 (also referred to as VEGFR3), VEGFR1, CXCR4, CD31, CD34, CD105, CD133, Sca-1, and c-Kit. For example, the Flk1+ cell may be Flk1+, Flk1+Flt1+, Flk1+Flt4+, Flk1+Flt1+Flt4+, Flk1+Flt1+VEGFR1+, or Flk1+Flt1+VEGFR1+CXCR4+, or any combinations comprising the additional markers listed above.

In some cases, hematopoietic EPCs may express cell surface markers selected from Flt1, Flt4, KDR1, VEGFR1, VEGFR2, CD309, CXCR4, CD31, CD34, CD105, CD133, Sca-1, and c-Kit. The hematopoietic EPCs may not express Flk1. The hematopoietic EPCs may express cell surface markers, such as CD34, Flt4, CD105, and CD133. As disclosed herein, hematopoietic EPCs could be endothelial precursor cells that express any combinations of the cell surface markers disclosed herein.

As used herein, a “biological sample” refers to bodily fluids and/or tissues including blood, plasma, serum, stool, lymph, cerebrospinal fluid, saliva, sputum, tears, sweat, semen, transudate, urine, exudate, tissue biopsy, and synovial fluid. In some embodiments, the sample comprises, consists essentially of, or consists of Flk1+ cells, or gene products produced from Flk1+ cells (for example fractions of Flk1+ cells, and/or proteins secreted by Flk1+ cells). In some embodiments, the sample comprises, consists essentially of, or consists of hematopoietic EPCs that does not express Flk1 but express cell surface markers selected from Flt1, Flt4, KDR1, VEGFR1, CXCR4, CD31, CD34, CD105, CD133, Sca-1, and c-Kit, or gene products produced from these cells (for example fractions of these cells, and/or proteins secreted by these cells). In some embodiments, the sample comprises, consists essentially of, or consists of whole Flk1+ cells as described herein. In some embodiments, the sample comprises, consists essentially of, or consists of whole hematopoietic EPCs that does not express Flk1, but express cell surface markers selected from Flt1, Flt4, KDR1, VEGFR1, CXCR4, CD31, CD34, CD105, CD133, Sca-1, and c-Kit, as described herein. In some embodiments, the sample comprises, consists essentially of, or consists of isolated Flk1+ cells. For example, at least 5%, 10%, 20%, 30%, 40%, 50%, 60%, 70%, 80%, 85%, 90%, 95%, 97%, or 99% of the cells in the biological sample can be Flk1+ cells, including ranges between any two of the listed values, for example, 5%-80%, 5%-99%, 20%-80%, 20%-99%, 50%-80%, or 50%-99%. In some embodiments, the sample comprises, consists essentially of, or consists of isolated hematopoietic EPCs that does not express Flk1, but express cell surface markers selected from Flt1, Flt4, KDR1, VEGFR1, CXCR4, CD31, CD34, CD105, CD133, Sca-1, and c-Kit. In some embodiments, single cells of the biological sample are analyzed. In some embodiments, the sample comprises, consists essentially of, or consists of a sample of isolated Flk1+ cells, or a fraction or cellular secretion of these cells. In some embodiments, the sample comprises, consists essentially of, or consists of a sample of isolated hematopoietic EPCs that does not express Flk1 but express cell surface markers selected from Flt1, Flt4, KDR1, VEGFR1, CXCR4, CD31, CD34, CD105, CD133, Sca-1, and c-Kit, or a fraction or cellular secretion of these cells. For example, the expression level of a nucleic acid sequence or a peptide sequence encoded therefrom of one or more of SEQ ID NOs: 1-5103, Genes Number 1-9280, or a peptide sequence encoded therefrom in the cellular secretions or fractions may be measured or detected.

To avoid or minimize contamination with gene products of other cells, the hematopoietic EPCs disclosed herein may be obtained as whole cells. However, once these whole cells have been isolated, cellular secretions or fractions may be used. Accordingly, in some embodiments, a sample comprises a cellular secretion or fraction of, or isolated hematopoietic EPCs disclosed herein. In some embodiments, the sample comprises one or more Flk1+ cells. In some embodiments, the sample can comprise at least 10⁴, 10⁵, 10⁶, 10⁷, 10⁸, 10⁹ or more (including ranges between any two of the listed values, for example 10⁴-10⁶, 10⁴-10⁹, or 10⁶-10⁹) hematopoietic EPCs or fractions or secretions thereof. Samples comprising hematopoietic EPCs (or fractions or secretions thereof) may also comprise body fluids and/or tissues (including but not limited to blood, plasma, serum, stool, lymph, cerebrospinal fluid, saliva, sputum, tears, sweat, semen, transudate, exudate, tissue biopsies and synovial fluid). The body fluid and/or tissue may comprise one or more hematopoietic EPCs, or fractions or sections of one more hematopoietic EPCs.

As used herein “isolated” hematopoietic EPC has its ordinary and customary meaning as would be understood by one of ordinary skill in the art in view of this disclosure. It refers to hematopoietic EPCs in a composition that is enriched for this cell type. The hematopoietic EPCs may be present in concentrations relative to other cell types sufficient for detection of expression profiles of gene products described herein associated with conditions comprising ischemia or neoangiogenesis. In methods, compositions, kits, and uses of some embodiments, the biological samples comprise at least 20%, 30%, 40%, 50%, 60%, 70%, 80%, 90%, 95%, or 99% isolated hematopoietic EPCs, fractions, or secretions thereof compared to other cell types, including ranges between any two of the listed values such as 20-99%, 20-90%, 20-80%, 40-99%, 40-90%, 40-80%, 50%-99%, 50%-90%, 50%-80%, 70%-99%, 70%-90%, 70%-80%, 90%-99%, 90%-95% or 95%-99%. In some embodiments, the biological samples consist essentially of or consist of hematopoietic EPCs, fractions, or secretions thereof. In some embodiments, the biological samples comprise isolated whole mononuclear peripheral blood cells that consists essentially of the hematopoietic EPCs. In some embodiments, the isolated hematopoietic EPCs are in a biological sample that is free of other cell types. Hematopoietic EPCs may be isolated using any suitable technique, for example, flow cytometry for cells positive for Flk1, Flt1, Flt4, CD31, CD34, or any other cell surface markers disclosed herein and/or separation of cells using antibodies for Flk1, Flt1, Flt4, CD31, CD34, or any other cell surface markers disclosed herein, affixed to a solid phase (so that cells comprising Flk1, Flt1, Flt4, CD31, CD34, or any other cell surface markers disclosed herein can be bound to the solid phase, while other cells are washed away or discarded). By way of example, the solid phase may comprise beads such as magnetic beads. By way of example, isolation of cells such as hematopoietic EPCs with Flk1 marker is described in U.S. Pat. No. 6,852,533 B1, which is hereby incorporated by reference in its entirety. Flk1+ cells may be isolated from biological samples such as blood samples. Flk1+ cells may be isolated from tissue biopsy samples. Cells can be isolated using methods such as (but not limited to) flow cytometry/FACS, Dynabeads magnetic beads, based on surface markers that are specifically expressed in the cells of interest.

Detecting a Condition Comprising Perfusion Shortage or a Risk Thereof with a High Accuracy

In some aspects, the present disclosure presents methods for detecting conditions comprising perfusion shortage or a risk thereof with an accuracy level of above at least 91%. As used herein, “accuracy” refers to the percentage of results that are neither false positives nor false negatives. Accuracy may be represented mathematically as 100%−((type 1 error)+(type 2 error)). Type 1 error may also be referred to as “alpha error.” Type 2 error may also be referred to as “beta error.”

Conventionally, patients analyzed for chest pain reminiscent of angina pectoris are analyzed using a combination of ECG, standard blood work, exercise testing, myocardial contrast echocardiography (MCE), CT analysis (including calcium imaging, CT angiography and MSCT), or more advanced perfusion imaging (including scintigraphy or nuclear imaging (PET, PET/CT, MIBI, SPECT, or CZT-SPECT)) or CMR perfusion imaging which has yielded an adequate diagnosis in 74% of the cases. Even with advanced perfusion imaging, the adequate/accurate diagnosis of ischemic heart disease does not exceed 84% using these conventional approaches. In addition, setting up a multi-disciplinary analysis of these patients remains a logistical challenge (involving functional testing and imaging, involvement of the departments of cardiology, nuclear medicine, and radiology) and represents a laborious and costly diagnostic track (with disappointing accuracy). In addition, with these conventional approaches, the extent of ischemia, or the biological response to ischemia cannot be readily quantified (or expressed in AU) for use in monitoring of these patients for disease progression or therapy responsiveness.

The present disclosure shows measuring or detecting signals from multiple genes (protein or nucleotide biomarkers) in accordance with some embodiments can yield superior accuracy for detecting conditions comprising perfusion shortage or a risk thereof. Based on the organism studied, biological samples containing cells expressing relevant markers (as such, it will be appreciated that for a human subject, a “Flk1+” cell refers to a “VEGFR2+” or “CD309” cell, and that in a mouse, a “Flk1+ cell refers to a “Flk1+” cell) are obtained for further analysis. These expression profiles can offer accurate identification, prediction, and monitoring of conditions comprising perfusion shortage, having substantially lower misclassification scores than conventional approaches. However, not all cell types exhibit gene product expression profiles indicative of such conditions or risks thereof, and the presence of expression data from other cell types can cause noise that interferes with identification, prediction, and monitoring of conditions comprising perfusion shortage. Thus, in accordance with methods, compositions, kits, and uses of some embodiments, gene expression profiles can be detected in isolated hematopoietic EPCs disclosed herein or fractions or secretions thereof, so as to avoid noise that may be caused by detection of expression profiles in other cell populations. Furthermore, it is observed that superior classification of conditions comprising perfusion shortage (or risk of the same) can be obtained when the number of different gene products utilized is within a certain range. Accordingly, in some embodiments, a condition comprising perfusion shortage, or a risk of such a condition is detected by detecting the expression level of a specified number (for example, at least 2, 3, 4, 5, 6, 7, 8, 9, 10, 11, or 12, or 4-40, 4-60, 6-40, 6-60, 8-40, 8-60, 10-40, or 10-60) different gene products selected from Tables 1-6. The expression levels can be detected in isolated subject hematopoietic EPCs disclosed herein or fractions or secretions thereof of the subject.

In some embodiments, when the subject is female, the expression levels of less than 60, 55, 50, 45, 40, 35, 30, 25, 20, 15, 14, 13, 12, 10, 8, 6, 4 or 2 genes (nucleotide or protein biomarkers) from the plurality of genes listed in Tables 1, 3, and 5 are measured and detected. In some cases, when the subject is female, the expression levels of less than 60, 55, 50, 45, 40, 35, 30, 25, 20, 15, 14, 13, 12, 10, 8, 6, 4 or 2 genes (nucleotide or protein biomarkers) from the plurality of genes listed in Table 1 are measured and detected. In some cases, when the subject is female, the expression levels of less than 60, 55, 50, 45, 40, 35, 30, 25, 20, 15, 14, 13, 12, 10, 8, 6, 4 or 2 genes (nucleotide or protein biomarkers) from the plurality of genes are selected from the group consisting of ZNF807, LOC100130331, LOC100130872, LINC01502, FAM66A, SRGAP3-AS3, MIR1185-1, MIR1266, MIR1261, MIR4262, MTRNR2L10, MIR3928, LOC100507065, LOC100507642, MIR1273F, MIR4519, MIR4799, MIR4747, PTPRU, LOC100996660, DNAL4, XXYLT1-AS2, LOC101927230, LOC101927377, LOC101927479, LOC101927503, LINC01186, LOC101927735, LOC101927880, LINC01486, LOC101928775, RHOXF1P1, LOC101929108, LOC101929330, LOC101929696, CEACAM5, LAMTOR5, CELP, SLC27A4, RCC1, STMN2, CAPN11, FDX1L, MIR155HG, CSMD2, PNMA5, HSPA12B, CLN5, LRRC38, COL3A1, ARHGEF19, PROKR2, COX6C, PTRH1, KRTAP13-1, ASB9, CRYGC, VTI1A, BEST3, SLC22A31, ADIG, LINC01104, PYDC2, PRSS3P2, OTUD7A, DCC, RBM46, TCEANC, ADAMTS17, EFNA5, CELSR2, U2AF1L4, PIKFYVE, KCTD6, PTPLB, EMX1, OR9I1, JAZF1, LAMB4, FOXC2, NCOA6, MESDC2, FBXO28, DNMBP, U2SURP, ABCA6, CBX5, CBLC, KIF4A, C20orf166-AS1, NUDT8, NUP210P1, SMCO1, MAP7D2, ALX3, FAM133B, SUN2, HECTD1, WIPI2, SNORD53, GJA8, DHDH, GPR4, MRGPRG-AS1, LOC283731, LINC00304, ZNF763, IGHV1-58, LOC284933, RBMY1A3P, GPX5, MED4, C6orf15, GUCY1A2, GUCA2A, HUNK, TFAP2E, POTEA, LEUTX, OR2T3, IGFALS, C19orf45, FAM99A, IGFL3, OR2M1P, FAM90A25P, C8orf86, TMEM8C, PABPN1L, LIG4, FLJ26245, KRT16P2, PRSS57, LTBP3, MIR215, ARSF, MEF2C, IQSEC3, LOC441179, ZNRF2P1, MT1M, MTIF2, MYO7A, NDUFB9, ATP2B1, MIR422A, CEND1, LARS, UFM1, PDHA2, CES1P1, PLTP, LURAP1, MRPS21, MED1, PPP1R3C, RBM41, ASIC4, ZNF821, PPP1R9A, TRMU, UTP6, CCDC177, TBC1D24, GPR158, NYNRIN, BAI2, GBA3, PTPRO, RAC3, CDH26, RPLPO, BGN, FAM204A, PJA1, MT1IP, SHBG, LOC646513, SLA, SKOR2, SLC34A1, SNRPD1, SOX9, SP1, MIR532, TDG, TNNI3, DNLZ, LOC729739, CBA, VIPR2, USP7, DDX39B, ICE2, DCSTAMP, HIST1H4J, ZNF484, REG4, LRRIQ1, FAM126A, DOCK7, RNMT, ST3GAL5, GAL3ST3, SLC25A21, FATE1, UNC5A, TBX18, FIBP, DLGAP1, MGME1, SYCE1, HAND1, TNFRSF8, MAP4K4, GOSR1, SETDB1, SLC23A2, DOPEY2, TBX5-AS1, MIR216B, LOC100128364, LOC100130093, LINC01387, PSORS1C3, LOC100131372, UBE2DNL, LOC100133331, SNAR-I, NCAM1-AS1, WI2-237311.2, LOC100288866, MIR1289-2, MIR1976, MIR1250, MIR3115, MIR3907, MIR3944, CCDC179, CEBPZOS, LOC100505940, LOC100506125, LOC100506713, NA, MIR378D1, MIR4489, NA, PQBP1, USP12-AS2, ARHGEF38-IT1, LINC01314, LOC100996637, LOC101060400, LRPPRC, LOC101926941, LOC101927029, LOC101927419, LOC101927542, LOC101927585, LOC101927722, MRGPRF-AS1, LOC101928765, LOC101928850, LOC101929074, LOC101929239, LAMP5-AS1, LOC101929374, NA, ENOX2, ARFGEF2, PROKR1, AFG3L2, GLIPR1, TLK2, INSL6, KLK8, VPS45, ATG4A, MRGPRE, C10orf71, C10orf32, ZFP3, CHMP4B, FAM83C, AP1S3, TRIM71, ADORA1, C9orf85, MAGEC3, SAMD10, C11orf40, SERPINA12, TMED6, MGAT5B, ZNRF4, C1orf64, FAM71A, LINC00896, CYP2A7, CYP2E1, C9orf163, RIBC1, ANKFN1, ZNF579, GPR125, MIER3, DEFB4A, C10orf91, COX7B2, DLX5, DNASE1L2, B3GNT6, LCE4A, LINC00870, OR8J1, OPN5, SCUBE3, FGF6, FGF11, ATG14, FOXD4, MYO16, DDN, FLNB, EHBP1, SLC16A8, FUT3, MYADML2, HIST1H2BA, GABRB3, NATD1, FSCN2, DFNB31, MTHFD1L, ANKRD2, RGS17, CACNG4, ARHGEF16, GOLGA1, GPR6, HECTD4, LINC00523, OR4N4, FADS6, LINC00482, SLC9C2, IGHV1-2, GPR25, RPL34-AS1, TRBV30, LCN12, IGLV6-57, ALG6, LMCD1, TLX3, RAX, HCRT, HMMR, HOXA7, APOA1, LOC338667, C2orf70, LOC339807, ZSCAN22, GPR149, MPC1L, SLC6A18, LINC00265, IL13, AQP5, KCNC3, ARF6, KRT81, LCN15, CES1P2, KRT39, OR7A10, LINC01121, LOC401127, CD180, MAT1A, LOC440300, FLJ31662, NDUFS1, Sep-02, NEUROD1, NOTCH4, COL5A3, BOLA1, DESI2, GAL, CDC40, EIF3L, COMMD10, PGF, PKP1, CPXCR1, PRKAG3, FXYD7, POU3F3, NUTM2F, PCSK4, VPS13C, EPS8L1, PPL, CCDC40, RBM23, TMEM38B, RIC8B, ETNK2, DDX19A, ZNF280C, WDR11, C1orf106, EAPP, USE1, ECHDC1, PCDHA6, TEX13B, PROC, KLK6, TBX20, CD177, PSMD13, TTYH1, CLK4, LRTM1, ZNF512B, PTK6, ADGRB2, PTN, VAT1L, IGDCC4, PBOV1, NYX, RPL15, RPS6, RPS21, SCT, CCL19, NECAB3, WIPF3, TMEM168, FLJ38668, LOC644961, EBF2, ANHX, ST3GAL4, BRD9, ELOVL4, MIR591, TF, TP73, C5, UBE2V2, UMOD, MIR802, ZNF226, SLC30A3, HSD17B8, TUBAL3, IRAPPC13, TMEM62, ACAD10, THUMPD2, WBSCR16, PVRL4, TKTL1, INHBE, ATAD3B, CASP7, GFM2, KRTAP4-4, TTBK1, RNASE7, CCDC54, MYO18B, ZSCAN10, FIBCD1, ATG4C, RERG, TCAP, DENR, AKR7A2, CADPS, CBFB, MBTPS1, MYH13, PER2, GYG2, CDK5R2, HIST1H3F, PAPLN, BAZ1B, CTU1, BTAF1, PIAS2, CERS5, INA, SYNGR3, ACBD5, SLFN11, BMP15, LGI1, MEX3A, DMKN, TCEAL1, LHX2, PPT2, NCR2, MEDT, CABP1, ADAMTS1, NUP155, ADAMTSL2, FAM115A, ZBTB39, HNRNPDL, and DMTF1. In some cases, when the subject is female, the expression levels of two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more genes, three hundred or more, four hundred or more, or five hundred or more (nucleotide or protein biomarkers) for the plurality of genes listed above are measured and detected.

In some cases, when the subject is female, the expression levels of less than 60, 55, 50, 45, 35, 30, 25, 20, 15, 14, 13, 12, 10, 8, 6, 4 or 2 genes (nucleotide or protein biomarkers) from the plurality of genes listed in Table 1 are measured and detected. In some cases, when the subject is female, the expression levels of less than 60, 55, 50, 45, 40, 35, 30, 25, 20, 15, 14, 13, 12, 10, 8, 6, 4 or 2 genes (nucleotide or protein biomarkers) from the plurality of genes are selected from the group consisting of ZNF807, LOC100130331, LOC100130872, LINC01502, FAM66A, SRGAP3-AS3, MIR1185-1, MIR1266, MIR1261, MIR4262, MTRNR2L10, MIR3928, LOC100507065, LOC100507642, MIR1273F, MIR4519, MIR4799, MIR4747, PTPRU, LOC100996660, DNAL4, XXYLT1-AS2, LOC101927230, LOC101927377, LOC101927479, LOC101927503, LINC01186, LOC101927735, LOC101927880, LINC01486, LOC101928775, RHOXF1P1, LOC101929108, LOC101929330, LOC101929696, CEACAM5, LAMTOR5, CELP, SLC27A4, RCC1, STMN2, CAPN11, FDX1L, MIR155HG, CSMD2, PNMA5, HSPA12B, CLN5, LRRC38, COL3A1, ARHGEF19, PROKR2, COX6C, PTRH1, KRTAP13-1, ASB9, CRYGC, VTI1A, BEST3, SLC22A31, ADIG, LINC01104, PYDC2, PRSS3P2, OTUD7A, DCC, RBM46, TCEANC, ADAMTS17, EFNA5, CELSR2, U2AF1L4, PIKFYVE, KCTD6, PTPLB, EMX1, OR9I1, JAZF1, LAMB4, FOXC2, NCOA6, MESDC2, FBXO28, DNMBP, U2SURP, ABCA6, CBX5, CBLC, KIF4A, C20orf166-AS1, NUDT8, NUP210P1, SMCO1, MAP7D2, ALX3, FAM133B, SUN2, HECTD1, WIPI2, SNORD53, GJA8, DHDH, GPR4, MRGPRG-AS1, LOC283731, LINC00304, ZNF763, IGHV1-58, LOC284933, RBMY1A3P, GPX5, MED4, C6orf15, GUCY1A2, GUCA2A, HUNK, TFAP2E, POTEA, LEUTX, OR2T3, IGFALS, C19orf45, FAM99A, IGFL3, OR2M1P, FAM90A25P, C8orf86, TMEM8C, PABPN1L, LIG4, FLJ26245, KRT16P2, PRSS57, LTBP3, MIR215, ARSF, MEF2C, IQSEC3, LOC441179, ZNRF2P1, MT1M, MTIF2, MYO7A, NDUFB9, ATP2B1, MIR422A, CEND1, LARS, UFM1, PDHA2, CES1P1, PLTP, LURAP1, MRPS21, MED1, PPP1R3C, RBM41, ASIC4, ZNF821, PPP1R9A, TRMU, UTP6, CCDC177, TBC1D24, GPR158, NYNRIN, BAI2, GBA3, PTPRO, RAC3, CDH26, RPLPO, BGN, FAM204A, PJA1, MT1IP, SHBG, LOC646513, SLA, SKOR2, SLC34A1, SNRPD1, SOX9, SP1, MIR532, TDG, TNNI3, DNLZ, LOC729739, CBA, VIPR2, USP7, DDX39B, ICE2, DCSTAMP, HIST1H4J, ZNF484, REG4, LRRIQ1, FAM126A, DOCK7, RNMT, ST3GAL5, GAL3ST3, SLC25A21, FATE1, UNC5A, TBX18, FIBP, DLGAP1, MGME1, SYCE1, HAND1, TNFRSF8, MAP4K4, GOSR1, SETDB1, SLC23A2, DOPEY2, and TBX5-AS1. In some cases, when the subject is female, the expression levels of two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, or two hundred or more genes (nucleotide or protein biomarkers) for the plurality of genes listed above are measured and detected.

In some cases, when the subject is female, the expression levels of less than 60, 55, 50, 45, 35, 30, 25, 20, 15, 14, 13, 12, 10, 8, 6, 4 or 2 genes (nucleotide or protein biomarkers) detected using the plurality of probes and/or mRNA targets listed in Table 3 are measured and detected. The probes and/or mRNA target sequences are designed to measure and/or detect certain genes. In some cases, the plurality of exon regions of genes selected from Table 5 are selected from the group of genes in Table 19. In some cases, when the subject is female, the expression levels of less than 60, 55, 50, 40, 35, 30, 25, 20, 15, 14, 13, 12, 10, 8, 6, 4 or 2 exon regions (nucleotide or protein biomarkers) from the plurality of exon regions of genes listed in Table 5 are measured and detected. In some cases, the plurality of exon regions of genes selected from Table 5 are selected from the group of genes in Table 20. In some cases, the plurality of exon regions of genes selected from Table 5 comprise at least one of the genes selected from the group consisting of Gene Number 1554, Gene Number 1558 (ARHGEF16), Gene Number 1606 (SAMD13), Gene Number 1782 (ELK4), Gene Number 1785 (ESRRG), Gene Number 1788, Gene Number 1790 (HHIPL2), Gene Number 1792 (TRIM17), Gene Number 1822 (GHITM), and Gene Number 1837 (BTRC). In some cases, the plurality of exon regions of genes selected from Table 5 comprise at least two of the genes selected from the group consisting of Gene Number 1554, Gene Number 1558 (ARHGEF16), Gene Number 1606 (SAMD13), Gene Number 1782 (ELK4), Gene Number 1785 (ESRRG), Gene Number 1788, Gene Number 1790 (HHIPL2), Gene Number 1792 (TRIM17), Gene Number 1822 (GHITM), and Gene Number 1837 (BTRC). In some cases, the plurality of exon regions of genes selected from Table 5 comprise at least three of the genes selected from the group consisting of Gene Number 1554, Gene Number 1558 (ARHGEF16), Gene Number 1606 (SAMD13), Gene Number 1782 (ELK4), Gene Number 1785 (ESRRG), Gene Number 1788, Gene Number 1790 (HHIPL2), Gene Number 1792 (TRIM17), Gene Number 1822 (GHITM), and Gene Number 1837 (BTRC). In some cases, the plurality of exon regions of genes selected from Table 5 comprise at least four of the genes selected from the group consisting of Gene Number 1554, Gene Number 1558 (ARHGEF16), Gene Number 1606 (SAMD13), Gene Number 1782 (ELK4), Gene Number 1785 (ESRRG), Gene Number 1788, Gene Number 1790 (HHIPL2), Gene Number 1792 (TRIM17), Gene Number 1822 (GHITM), and Gene Number 1837 (BTRC). In some cases, the plurality of exon regions of genes selected from Table 5 comprise at least five of the genes selected from the group consisting of Gene Number 1554, Gene Number 1558 (ARHGEF16), Gene Number 1606 (SAMD13), Gene Number 1782 (ELK4), Gene Number 1785 (ESRRG), Gene Number 1788, Gene Number 1790 (HHIPL2), Gene Number 1792 (TRIM17), Gene Number 1822 (GHITM), and Gene Number 1837 (BTRC). In some cases, the plurality of exon regions of genes selected from Table 5 comprise at least six of the genes selected from the group consisting of Gene Number 1554, Gene Number 1558 (ARHGEF16), Gene Number 1606 (SAMD13), Gene Number 1782 (ELK4), Gene Number 1785 (ESRRG), Gene Number 1788, Gene Number 1790 (HHIPL2), Gene Number 1792 (TRIM17), Gene Number 1822 (GHITM), and Gene Number 1837 (BTRC). In some cases, the plurality of exon regions of genes selected from Table 5 comprise Gene Number 1554, Gene Number 1558 (ARHGEF16), Gene Number 1606 (SAMD13), Gene Number 1782 (ELK4), Gene Number 1785 (ESRRG), Gene Number 1788, Gene Number 1790 (HHIPL2), Gene Number 1792 (TRIM17), Gene Number 1822 (GHITM), and Gene Number 1837 (BTRC). In some cases, the plurality of exon regions of genes selected from Table 5 consists of Gene Number 1554, Gene Number 1558 (ARHGEF16), Gene Number 1606 (SAMD13), Gene Number 1782 (ELK4), Gene Number 1785 (ESRRG), Gene Number 1788, Gene Number 1790 (HHIPL2), Gene Number 1792 (TRIM17), Gene Number 1822 (GHITM), and Gene Number 1837 (BTRC).

In some cases, the plurality of exon regions of genes selected from Table 5 comprise at least one of the genes selected from the group consisting of Gene Number 1562 (CLCN6), Gene Number 1576 (EXTL1), Gene Number 1577 (ATPIF1), Gene Number 1580 (GJB3), Gene Number 1591, Gene Number 1606 (SAMD13), Gene Number 1626 (VPS45), Gene Number 1658 (KCTD3), Gene Number 1693 and (AIM1L). In some cases, the plurality of exon regions of genes selected from Table 5 comprise at least two of the genes selected from the group consisting of Gene Number 1562 (CLCN6), Gene Number 1576 (EXTL1), Gene Number 1577 (ATPIF1), Gene Number 1580 (GJB3), Gene Number 1591, Gene Number 1606 (SAMD13), Gene Number 1626 (VPS45), Gene Number 1658 (KCTD3), Gene Number and 1693 (AIM1L). In some cases, the plurality of exon regions of genes selected from Table 5 comprise at least three of the genes selected from the group consisting of Gene Number 1562 (CLCN6), Gene Number 1576 (EXTL1), Gene Number 1577 (ATPIF1), Gene Number 1580 (GJB3), Gene Number 1591, Gene Number 1606 (SAMD13), Gene Number 1626 (VPS45), Gene Number 1658 (KCTD3), and Gene Number 1693 (AIM1L). In some cases, the plurality of exon regions of genes selected from Table 5 comprise at least four of the genes selected from the group consisting of Gene Number 1562 (CLCN6), Gene Number 1576 (EXTL1), Gene Number 1577 (ATPIF1), Gene Number 1580 (GJB3), Gene Number 1591, Gene Number 1606 (SAMD13), Gene Number 1626 (VPS45), Gene Number 1658 (KCTD3), and Gene Number 1693 (AIM1L). In some cases, the plurality of exon regions of genes selected from Table 5 comprise at least five of the genes selected from the group consisting of Gene Number 1562 (CLCN6), Gene Number 1576 (EXTL1), Gene Number 1577 (ATPIF1), Gene Number 1580 (GJB3), Gene Number 1591, Gene Number 1606 (SAMD13), Gene Number 1626 (VPS45), Gene Number 1658 (KCTD3), and Gene Number 1693 (AIM1L). In some cases, the plurality of exon regions of genes selected from Table 5 comprise at least six of the genes selected from the group consisting of Gene Number 1562 (CLCN6), Gene Number 1576 (EXTL1), Gene Number 1577 (ATPIF1), Gene Number 1580 (GJB3), Gene Number 1591, Gene Number 1606 (SAMD13), Gene Number 1626 (VPS45), Gene Number 1658 (KCTD3), and Gene Number 1693 (AIM1L). In some cases, the plurality of exon regions of genes selected from Table 5 comprise Gene Number 1562 (CLCN6), Gene Number 1576 (EXTL1), Gene Number 1577 (ATPIF1), Gene Number 1580 (GJB3), Gene Number 1591, Gene Number 1606 (SAMD13), Gene Number 1626 (VPS45), Gene Number 1658 (KCTD3), and Gene Number 1693 (AIM1L). In some cases, the plurality of exon regions of genes selected from Table 5 consist of Gene Number 1562 (CLCN6), Gene Number 1576 (EXTL1), Gene Number 1577 (ATPIF1), Gene Number 1580 (GJB3), Gene Number 1591, Gene Number 1606 (SAMD13), Gene Number 1626 (VPS45), Gene Number 1658 (KCTD3), and Gene Number 1693 (AIM1L).

In some cases, the plurality of exon regions of genes selected from Table 5 comprise at least one gene selected from the group consisting of Gene Number 1553 (AGRN), Gene Number 1555, Gene Number 1565 (PDPN), Gene Number 1573 (C1orf213), Gene Number 1576 (EXTL1), Gene Number 1588 (KIF2C), Gene Number 1620 (MAN1A2), Gene Number 1636 (USP21), and Gene Number 1770. In some cases, the plurality of exon regions of genes selected from Table 5 comprise at least two genes selected from the group consisting of Gene Number 1553 (AGRN), Gene Number 1555, Gene Number 1565 (PDPN), Gene Number 1573 (C1orf213), Gene Number 1576 (EXTL1), Gene Number 1588 (KIF2C), Gene Number 1620 (MAN1A2), Gene Number 1636 (USP21), and Gene Number 1770. In some cases, the plurality of exon regions of genes selected from Table 5 comprise at least three genes selected from the group consisting of Gene Number 1553 (AGRN), Gene Number 1555, Gene Number 1565 (PDPN), Gene Number 1573 (C1orf213), Gene Number 1576 (EXTL1), Gene Number 1588 (KIF2C), Gene Number 1620 (MAN1A2), Gene Number 1636 (USP21), and Gene Number 1770. In some cases, the plurality of exon regions of genes selected from Table 5 comprise at least four genes selected from the group consisting of Gene Number 1553 (AGRN), Gene Number 1555, Gene Number 1565 (PDPN), Gene Number 1573 (C1orf213), Gene Number 1576 (EXTL1), Gene Number 1588 (KIF2C), Gene Number 1620 (MAN1A2), Gene Number 1636 (USP21), and Gene Number 1770. In some cases, the plurality of exon regions of genes selected from Table 5 comprise at least five genes selected from the group consisting of Gene Number 1553 (AGRN), Gene Number 1555, Gene Number 1565 (PDPN), Gene Number 1573 (C1orf213), Gene Number 1576 (EXTL1), Gene Number 1588 (KIF2C), Gene Number 1620 (MAN1A2), Gene Number 1636 (USP21), and Gene Number 1770. In some cases, the plurality of exon regions of genes selected from Table 5 comprise at least six genes selected from the group consisting of Gene Number 1553 (AGRN), Gene Number 1555, Gene Number 1565 (PDPN), Gene Number 1573 (C1orf213), Gene Number 1576 (EXTL1), Gene Number 1588 (KIF2C), Gene Number 1620 (MAN1A2), Gene Number 1636 (USP21), and Gene Number 1770. In some cases, the plurality of exon regions of genes selected from Table 5 comprise Gene Number 1553 (AGRN), Gene Number 1555, Gene Number 1565 (PDPN), Gene Number 1573 (C1orf213), Gene Number 1576 (EXTL1), Gene Number 1588 (KIF2C), Gene Number 1620 (MAN1A2), Gene Number 1636 (USP21), and Gene Number 1770. In some cases, the plurality of exon regions of genes selected from Table 5 consist of Gene Number 1553 (AGRN), Gene Number 1555, Gene Number 1565 (PDPN), Gene Number 1573 (C1orf213), Gene Number 1576 (EXTL1), Gene Number 1588 (KIF2C), Gene Number 1620 (MAN1A2), Gene Number 1636 (USP21), and Gene Number 1770.

In some cases, the plurality of exon regions of genes selected from Table 5 comprise at least one gene selected from the group consisting of Gene Number 1555, Gene Number 1562 (CLCN6), Gene Number 1566 (PRDM2), Gene Number 1616, Gene Number 1627 (SETDB1), Gene Number 1648 (CAMSAP2), Gene Number 2729 (ZNF765), Gene Number 2739 (GPR108), Gene Number 2764, Gene Number 2824 (C2orf15), Gene Number 2879 (SPEG), Gene Number 2890 (SEPT2), and Gene Number 2892. In some cases, the plurality of exon regions of genes selected from Table 5 comprise at least two genes selected from the group consisting of Gene Number 1555, Gene Number 1562 (CLCN6), Gene Number 1566 (PRDM2), Gene Number 1616, Gene Number 1627 (SETDB1), Gene Number 1648 (CAMSAP2), Gene Number 2729 (ZNF765), Gene Number 2739 (GPR108), Gene Number 2764, Gene Number 2824 (C2orf15), Gene Number 2879 (SPEG), Gene Number 2890 (SEPT2), and Gene Number 2892. In some cases, the plurality of exon regions of genes selected from Table 5 comprise at least three genes selected from the group consisting of Gene Number 1555, Gene Number 1562 (CLCN6), Gene Number 1566 (PRDM2), Gene Number 1616, Gene Number 1627 (SETDB1), Gene Number 1648 (CAMSAP2), Gene Number 2729 (ZNF765), Gene Number 2739 (GPR108), Gene Number 2764, Gene Number 2824 (C2orf15), Gene Number 2879 (SPEG), Gene Number 2890 (SEPT2), and Gene Number 2892. In some cases, the plurality of exon regions of genes selected from Table 5 comprise at least four genes selected from the group consisting of Gene Number 1555, Gene Number 1562 (CLCN6), Gene Number 1566 (PRDM2), Gene Number 1616, Gene Number 1627 (SETDB1), Gene Number 1648 (CAMSAP2), Gene Number 2729 (ZNF765), Gene Number 2739 (GPR108), Gene Number 2764, Gene Number 2824 (C2orf15), Gene Number 2879 (SPEG), Gene Number 2890 (SEPT2), and Gene Number 2892. In some cases, the plurality of exon regions of genes selected from Table 5 comprise at least five genes selected from the group consisting of Gene Number 1555, Gene Number 1562 (CLCN6), Gene Number 1566 (PRDM2), Gene Number 1616, Gene Number 1627 (SETDB1), Gene Number 1648 (CAMSAP2), Gene Number 2729 (ZNF765), Gene Number 2739 (GPR108), Gene Number 2764, Gene Number 2824 (C2orf15), Gene Number 2879 (SPEG), Gene Number 2890 (SEPT2), and Gene Number 2892. In some cases, the plurality of exon regions of genes selected from Table 5 comprise at least six genes selected from the group consisting of Gene Number 1555, Gene Number 1562 (CLCN6), Gene Number 1566 (PRDM2), Gene Number 1616, Gene Number 1627 (SETDB1), Gene Number 1648 (CAMSAP2), Gene Number 2729 (ZNF765), Gene Number 2739 (GPR108), Gene Number 2764, Gene Number 2824 (C2orf15), Gene Number 2879 (SPEG), Gene Number 2890 (SEPT2), and Gene Number 2892. In some cases, the plurality of exon regions of genes selected from Table 5 comprise Gene Number 1555, Gene Number 1562 (CLCN6), Gene Number 1566 (PRDM2), Gene Number 1616, Gene Number 1627 (SETDB1), Gene Number 1648 (CAMSAP2), Gene Number 2729 (ZNF765), Gene Number 2739 (GPR108), Gene Number 2764, Gene Number 2824 (C2orf15), Gene Number 2879 (SPEG), Gene Number 2890 (SEPT2), and Gene Number 2892. In some cases, the plurality of exon regions of genes selected from Table 5 consist of Gene Number 1555, Gene Number 1562 (CLCN6), Gene Number 1566 (PRDM2), Gene Number 1616, Gene Number 1627 (SETDB1), Gene Number 1648 (CAMSAP2), Gene Number 2729 (ZNF765), Gene Number 2739 (GPR108), Gene Number 2764, Gene Number 2824 (C2orf15), Gene Number 2879 (SPEG), Gene Number 2890 (SEPT2), and Gene Number 2892.

In some cases, the plurality of exon regions of genes selected from Table 5 comprise at least one gene selected from the group consisting of Gene Number 1573 (C1orf213), Gene Number 1636 (USP21), Gene Number 1639 (DDR2), Gene Number 1778 (LOC730227), Gene Number 2869 (NRP2), Gene Number 3666 (HLA-A), Gene Number 3689 (SYNGAP1), Gene Number 3694, Gene Number 3710 (HOXA-AS2), and Gene Number 3781 (NPC1L1). In some cases, the plurality of exon regions of genes selected from Table 5 comprise at least two genes selected from the group consisting of Gene Number 1573 (C1orf213), Gene Number 1636 (USP21), Gene Number 1639 (DDR2), Gene Number 1778 (LOC730227), Gene Number 2869 (NRP2), Gene Number 3666 (HLA-A), Gene Number 3689 (SYNGAP1), Gene Number 3694, Gene Number 3710 (HOXA-AS2), and Gene Number 3781 (NPC1L1). In some cases, the plurality of exon regions of genes selected from Table 5 comprise at least three genes selected from the group consisting of Gene Number 1573 (C1orf213), Gene Number 1636 (USP21), Gene Number 1639 (DDR2), Gene Number 1778 (LOC730227), Gene Number 2869 (NRP2), Gene Number 3666 (HLA-A), Gene Number 3689 (SYNGAP1), Gene Number 3694, Gene Number 3710 (HOXA-AS2), and Gene Number 3781 (NPC1L1). In some cases, the plurality of exon regions of genes selected from Table 5 comprise at least four genes selected from the group consisting of Gene Number 1573 (C1orf213), Gene Number 1636 (USP21), Gene Number 1639 (DDR2), Gene Number 1778 (LOC730227), Gene Number 2869 (NRP2), Gene Number 3666 (HLA-A), Gene Number 3689 (SYNGAP1), Gene Number 3694, Gene Number 3710 (HOXA-AS2), and Gene Number 3781 (NPC1L1). In some cases, the plurality of exon regions of genes selected from Table 5 comprise at least five genes selected from the group consisting of Gene Number 1573 (C1orf213), Gene Number 1636 (USP21), Gene Number 1639 (DDR2), Gene Number 1778 (LOC730227), Gene Number 2869 (NRP2), Gene Number 3666 (HLA-A), Gene Number 3689 (SYNGAP1), Gene Number 3694, Gene Number 3710 (HOXA-AS2), and Gene Number 3781 (NPC1L1). In some cases, the plurality of exon regions of genes selected from Table 5 comprise at least six genes selected from the group consisting of Gene Number 1573 (C1orf213), Gene Number 1636 (USP21), Gene Number 1639 (DDR2), Gene Number 1778 (LOC730227), Gene Number 2869 (NRP2), Gene Number 3666 (HLA-A), Gene Number 3689 (SYNGAP1), Gene Number 3694, Gene Number 3710 (HOXA-AS2), and Gene Number 3781 (NPC1L1). In some cases, the plurality of exon regions of genes selected from Table 5 comprise Gene Number 1573 (C1orf213), Gene Number 1636 (USP21), Gene Number 1639 (DDR2), Gene Number 1778 (LOC730227), Gene Number 2869 (NRP2), Gene Number 3666 (HLA-A), Gene Number 3689 (SYNGAP1), Gene Number 3694, Gene Number 3710 (HOXA-AS2), and Gene Number 3781 (NPC1L1). In some cases, the plurality of exon regions of genes selected from Table 5 consist of Gene Number 1573 (C1orf213), Gene Number 1636 (USP21), Gene Number 1639 (DDR2), Gene Number 1778 (LOC730227), Gene Number 2869 (NRP2), Gene Number 3666 (HLA-A), Gene Number 3689 (SYNGAP1), Gene Number 3694, Gene Number 3710 (HOXA-AS2), and Gene Number 3781 (NPC1L1).

Further, the expression levels may be compared to control ranges disclosed here to generate an output/report indicative of the subject's having or risk of having a condition comprising perfusion shortage with an accuracy greater than about 99%. In some embodiments, the accuracy is about 99.9%, 99.8%, 99.7%, 99.6%, 99.5%, 99.4%, 99.3%, 99.2%, 99.1%, 98.9%, 98.5%, 98%, 97.5%, 97%, 96.5%, 96%, 95.5%, 95%, 94.5%, 94%, 93.5%, 93%, 92.5%, 92%, or 91.5%.

TABLE 19

| Code | | |
|---|---|---|
| PSC | biomarker nucleotide seq probe | biomarker nucleotide seq target mRNA |
| 2102 | ATCAGAGGCAGTTAAGATCCAATGT | TAGTCTCCGTCAATTCTAGGTTACA |
| 8673 | AAGTATAGACGCCCCGTCTCTGCCT | TTCATATCTGCGGGGCAGAGACGGA |
| 11464 | CTTTATCCCATTTTCGACGTGCCCG | GAAATAGGGTAAAAGCTGCACGGGC |
| 16663 | CTGAGACTCCCAGTACGCGGATCGT | GACTCTGAGGGTCATGCGCCTAGCA |
| 21817 | ACTCTCTTGCCAGTCGAACCTCGTG | TGAGAGAACGGTCAGCTTGGAGCAC |
| 24286 | NA | NA |
| 25439 | CGTGTAAATCCCACCGTACCGGAGA | GCACATTTAGGGTGGCATGGCCTCT |
| 29371 | AGGCACACTCCCAAGTTCCGGTTCG | TCCGTGTGAGGGTTCAAGGCCAAGC |
| 30491 | AGGCCTGTCCCAACTCCATCGAGAG | TCCGGACAGGGTTGAGGTAGCTCTC |
| 31618 | AGTGCCTGTCCCTATCTCCAAACGG | TCACGGACAGGGATAGAGGTTTGCC |
| 32943 | CTGTTCTCGTCCCTCGGTGTAGGAC | GACAAGAGCAGGGAGCCACATCCTG |
| 33806 | TCCGGAATCCAGAAGATAACAGTCT | AGGCCTTAGGTCTTCTATTGTCAGA |
| 36050 | GTTACGGTTTCCAGTCAGATCCAGG | CAATGCCAAAGGTCAGTCTAGGTCC |
| 40648 | ACCATACTTCCACGACCGGTGTCGG | TGGTATGAAGGTGCTGGCCACAGCC |
| 44512 | CCGCCGTCGTCCAATTCCTGTGAAA | GGCGGCAGCAGGTTAAGGACACTTT |
| 48445 | GTATAATGACCCTTATTTACTCCAG | CATATTACTGGGAATAAATGAGGTC |
| 52624 | AGTCTACGGTCCCTCCTATTTCGTC | TCAGATGCCAGGGAGGATAAAGCAG |
| 53091 | CTACAACTCCCTCGTGTAAACACAA | GATGTTGAGGGAGCACATTTGTGTT |
| 53355 | ACGACTGTTCCCTCTTAGTGGTGTC | TGCTGACAAGGGAGAATCACCACAG |
| 59716 | ACGTCGGTGACCCTAAGTCGAAGGG | TGCAGCCACTGGGATTCAGCTTCCC |
| 62038 | ACAAGAGATCCTGACCCTCTTCGAC | TGTTCTCTAGGACTGGGAGAAGCTG |
| 65216 | AAAGACGTCTCCTTTGCCCTACCAA | TTTCTGCAGAGGAAACGGGATGGTT |
| 65253 | CTCAGGATCCTGGTAGACCCGAGAC | GAGTCCTAGGACCATCTGGGCTCTG |
| 66201 | GGGCTCTCTCCTTCACCAGTCGATT | CCCGAGAGAGGAAGTGGTCAGCTAA |
| 71233 | ATTCCGGTCCTGTCTCTCAAATTAC | TAAGGCCAGGACAGAGAGTTTAATG |
| 88970 | AGGTGCCACCAGTCCATGTACTACG | TCCACGGTGGTCAGGTACATGATGC |
| 89503 | GGAATAAACCACTAAACTCGGAGAC | CCTTATTTGGTGATTTGAGCCTCTG |
| 98630 | GAGGTGACAGTACCACCCGAGGACA | CTCCACTGTCATGGTGGGCTCCTGT |
| 112599 | GTTCTTTCTACCTACCCTTTTGAGG | CAAGAAAGATGGATGGGAAAACTCC |
| 121129 | CCGAGTTTCCGATATCTTTATGGTG | GGCTCAAAGGCTATAGAAATACCAC |
| 143577 | AACGGTGCACCGTGTCGGAAGGTTT | TTGCCACGTGGCACAGCCTTCCAAA |
| 149915 | AGGTCTTGGGAGTTCTTATTAGGGA | TCCAGAACCCTCAAGAATAATCCCT |
| 151536 | TTAGGACCGGGGTATCGGACTCCCT | AATCCTGGCCCCATAGCCTGAGGGA |
| 156137 | ATAAATAGGGCTAGAAACTTGTGGT | TATTTATCCCGATCTTTGAACACCA |
| 175079 | AAAACAGTGGCAGGTCTTAGTGACC | TTTTGTCACCGTCCAGAATCACTGG |
| 179722 | ATCCGACATGGACCGCGTCTCCGGA | TAGGCTGTACCTGGCGCAGAGGCCT |
| 196695 | ATTTTTGGTGGATGACCGATGGTAC | TAAAAACCACCTACTGGCTACCATG |
| 200539 | ACGTCAATTGGTCATAAACTCCAGA | TGCAGTTAACCAGTATTTGAGGTCT |
| 202647 | AAATTGTGGTTGAGTCGTTCTCTAG | TTTAACACCAACTCAGCAAGAGATC |
| 232609 | AGAGTCCCTAGGATCACAGACCATC | TCTCAGGGATCCTAGTGTCTGGTAG |
| 235679 | GACTTAGTAGAAGGTTTTCTAAAGA | CTGAATCATCTTCCAAAAGATTTCT |
| 239356 | AGCTCCAGAGGTTCACCCTGTTTGG | TCGAGGTCTCCAAGTGGGACAAACC |
| 239483 | ACAGGTGAAGGTCTTTTCTCGGCGG | TGTCCACTTCCAGAAAAGAGCCGCC |
| 241691 | CATGTATACGAGGTGGAATTGGAAC | GTACATATGCTCCACCTTAACCTTG |
| 243938 | TTGGTACTGAGGTAAACAGTCAGAC | AACCATGACTCCATTTGTCAGTCTG |
| 279663 | ATATAAAACGGTAGCGAGTGGGCTT | TATATTTTGCCATCGCTCACCCGAA |
| 433854 | TTACCGGAGAAGAGCTAAGACGTTG | AATGGCCTCTTCTCGATTCTGCAAC |
| 449476 | CTCCAAGAAGTTTGAGGCCCGTATT | GAGGTTCTTCAAACTCCGGGCATAA |
| 508151 | GTCAGGTATTACAACATCAATGCCT | CAGTCCATAATGTTGTAGTTACGGA |
| 617278 | ATAACGCCCACTACTACTGTCCGAG | TATTGCGGGTGATGATGACAGGCTC |
| 680209 | ATACTGACTAAGAACGGCAACTCTG | TATGACTGATTCTTGCCGTTGAGAC |
| 854711 | CTGGTTTTTACTGTCCCGTTTCGCT | GACCAAAAATGACAGGGCAAAGCGA |
| 1279720 | GTTATTATTTATTCCCACTGCCCGA | CAATAATAAATAAGGGTGACGGGCT |
| 1326871 | TTGTGTACCCTAGATCCCTGACAGT | AACACATGGGATCTAGGGACTGTCA |

TABLE 20

| Affymetrix | Transcript | | | | |
|---|---|---|---|---|---|
| Exon ID B | Cluster ID | SEQ Name | Strand | Strand Start | Strand Stop |
| 16650141 | 16650141 | — | — | — | — |
| 16650467 | 16650467 | — | — | — | — |
| 16650951 | 16650951 | — | — | — | — |
| 16650957 | 16650957 | — | — | — | — |
| 16651195 | 16651195 | — | — | — | — |
| 16651411 | 16651411 | — | — | — | — |
| 16651571 | 16651571 | — | — | — | — |
| 16651811 | 16651811 | — | — | — | — |
| 16651885 | 16651885 | — | — | — | — |
| 16652173 | 16652173 | — | — | — | — |
| 16653133 | 16653133 | — | — | — | — |
| 16653857 | 16653857 | — | — | — | — |
| 16654419 | 16654419 | — | — | — | — |
| 16654495 | 16654495 | — | — | — | — |
| 16655639 | 16655639 | — | — | — | — |
| 16656867 | 16656867 | — | — | — | — |
| 16656939 | 16656939 | — | — | — | — |
| 16657644 | 16657598 | chr1 | + | 990376.00 | 990400.00 |
| 16657765 | 16657764 | chr1 | + | 1363700.00 | 1364109.00 |
| 16657951 | 16657948 | chr1 | + | 1982386.00 | 1982410.00 |
| 16658183 | 16658159 | chr1 | + | 3397184.00 | 3397501.00 |
| 16659119 | 16659102 | chr1 | + | 11893647.00 | 11893671.00 |
| 16659462 | 16659459 | chr1 | + | 13910596.00 | 13910623.00 |
| 16659488 | 16659478 | chr1 | + | 14095565.00 | 14095589.00 |
| 16660661 | 16660654 | chr1 | + | 23697718.00 | 23697938.00 |
| 16661046 | 16661031 | chr1 | + | 26361797.00 | 26361821.00 |
| 16661535 | 16661533 | chr1 | + | 28562744.00 | 28562770.00 |
| 16662326 | 16662322 | chr1 | + | 35250766.00 | 35250799.00 |
| 16663974 | 16663958 | chr1 | + | 45223239.00 | 45223263.00 |
| 16664693 | 16664690 | chr1 | + | 52518107.00 | 52518332.00 |
| 16666650 | 16666640 | chr1 | + | 84791390.00 | 84791414.00 |
| 16667753 | 16667752 | chr1 | + | 101616117.00 | 101616433.00 |
| 16669265 | 16669255 | chr1 | + | 118008956.00 | 118009010.00 |
| 16670417 | 16670404 | chr1 | + | 150049779.00 | 150049803.00 |
| 16670661 | 16670632 | chr1 | + | 150933479.00 | 150933503.00 |
| 16672757 | 16672754 | chr1 | + | 161130244.00 | 161130268.00 |
| 16673093 | 16673075 | chr1 | + | 162740364.00 | 162740388.00 |
| 16675168 | 16675158 | chr1 | + | 186280181.00 | 186280231.00 |
| 16675682 | 16675673 | chr1 | + | 200811130.00 | 200811197.00 |
| 16677481 | 16677473 | chr1 | + | 215752419.00 | 215752443.00 |
| 16683833 | 16683817 | chr1 | − | 26663713.00 | 26663737.00 |
| 16697485 | 16697484 | chr1 | − | 195727752.00 | 195728362.00 |
| 16698228 | 16698226 | chr1 | − | 203270218.00 | 203270406.00 |
| 16698507 | 16698504 | chr1 | − | 205569009.00 | 205569033.00 |
| 16699306 | 16699290 | chr1 | − | 216896744.00 | 216896768.00 |
| 16699434 | 16699433 | chr1 | − | 220160865.00 | 220160943.00 |
| 16699580 | 16699574 | chr1 | − | 222700336.00 | 222700389.00 |
| 16700232 | 16700218 | chr1 | − | 228604260.00 | 228604398.00 |
| 16706718 | 16706712 | chr10 | + | 85902423.00 | 85902496.00 |
| 16708482 | 16708468 | chr10 | + | 103295201.00 | 103295225.00 |
| 16864956 | 16864946 | chr19 | + | 53928352.00 | 53928381.00 |
| 16867866 | 16867837 | chr19 | − | 6737283.00 | 6737307.00 |
| 16871804 | 16871800 | chr19 | − | 37996874.00 | 37996940.00 |
| 16883383 | 16883380 | chr2 | + | 99758755.00 | 99758852.00 |
| 16889881 | 16889879 | chr2 | + | 206546738.00 | 206546762.00 |
| 16891318 | 16891273 | chr2 | + | 220341679.00 | 220341703.00 |
| 16893475 | 16893449 | chr2 | + | 242283265.00 | 242283289.00 |
| 16894005 | 16894001 | chr2 | − | 3581378.00 | 3581429.00 |
| 17037682 | 17037670 | chr6_qbl_hap6 | + | 1205278.00 | 1205318.00 |
| 17041405 | 17041393 | chr6 ssto_hap7 | + | 4886478.00 | 4886502.00 |
| 17042074 | 17042060 | chr6_ssto_hap7 | − | 3080352.00 | 3080376.00 |
| 17044504 | 17044491 | chr7 | + | 27163662.00 | 27163824.00 |
| 17057318 | 17057305 | chr7 | − | 44571379.00 | 44571428.00 |

In some embodiments, when the subject is male, the expression levels of less than 40, 35, 30, 25, 20, or 15 genes (nucleotide or protein biomarker) from said plurality of genes (nucleotide or protein sequences) listed in Tables 2, 4, and 6 are measured and detected. Further, the expression levels are compared to control ranges disclosed here to generate an output/report indicative of the subject's having or risk of having a condition comprising perfusion shortage with an accuracy greater than about 98%. In some embodiments, when the subject is male, the expression levels of less than 40, 35, 30, 25, 20, or 15 genes (nucleotide or protein biomarker) from said plurality of genes (nucleotide or protein sequences) listed in Table 2 are measured and detected. In some cases, when the subject is male, the expression levels of less than 40, 35, 30, 25, 20, or 15 genes (nucleotide or protein biomarkers) selected from the group of genes consisting of SNORD115-37, SNORD115-24, SNORA11B, MIR876, COL4A2-AS2, LOC100130373, LOC100130548, LOC100131496, LINC00240, BCL2L11, MED16, ERVV-2, KRTAP21-3, LOC100289230, LOC100289283, MIR1275, MIR1912, MIR1302-7, MIR1231, MIR1238, MIR1179, MIR1908, MIR1237, MIR2116, CSN1S2BP, MIR3148, MIR4280, MIR3116-1, MIR3129, MIR4275, MIR4326, MIR4254, LINC00673, MIR3926-2, MIR3678, MIR548Z, MIR3657, MIR3935, LOC100505478, LINC01364, C16orf95, LINC00837, MIR3529, MIR4475, MIR4492, MIR4784, MIR4732, MIR4684, MIR4773-2, MIR4429, MIR4442, LINC00507, LOC100996345, CYYR1-AS1, LOC100996635, CDK4, LOC101927053, PDZRN3-AS1, MEOX2-AS1, LINC01186, NA, GBAT2, LINC01214, LOC101928101, LOC101928118, NA, LOC101928195, LOC101928385, ICONS 00029157, LOC101928668, LINC01450, NA, LOC101928823, LOC101928919, LINC01262, LOC101929031, LOC101929159, LOC101929468, LOC101929497, ZNF267, CHERP, IFI44, FUT9, RFPL2, IFI44L, MAPRE2, SLC27A5, CHD1, RABL2B, FSTL1, MRPL3, GLB1L3, GPR182, CHRM5, SMIM12, LRRC37B, SMYD4, TBCB, KLHDC3, RPL39L, CLTB, PRSS30P, PCP2, FITM2, SNTN, TMEM68, OTUD6A, CRP, DYNLL2, CRYGD, NA, TMC8, CSTB, SLC35F3, CCDC140, WDSUB1, SH3D19, TEX28, FEZF1-AS1, ESCO2, ZNF645, CYP51A1, DAB1, ZCCHC12, DNMT3A, DRP2, EGR2, ABCA2, CDY2B, SENP5, EVI2B, ALCAM, ABCD1, F12, FAH, OR5A2, CCDC89, FABP5P3, FGF4, FGF14, DIP2A, ATP11B, NUP210, EXOC6B, ARC, SASH1, GRIP1, SEC61G, SCRIB, POLA2, GABARAPL1, DEFB108B, DEFB113, FTH1P3, CDRT15L2, HEATR9, ALS2CL, ASPM, OR2F1, GATA2, PLA2G2D, GCNT1, AGO1, OR7A5, OR1J2, SNORA70, GNAI3, BCL9L, OTOGL, SLC46A3, EWSAT1, LINC00927, C16orf54, MAMSTR, MCHR1, LOC284825, IGHD2-21, IGHD2-15, EOGT, C8orf31, IGLV10-54, IGLV3-12, SNX24, ARHGAP35, SCG3, PYCR2, SLC39A3, H3F3B, ANXA5, CXXC1, CDR2L, HNF4A, HNRNPK, SNORD56B, KRTAP20-1, KRTAP20-2, LOC339166, ZFP69, LOC340074, KIAA1875, OR2T8, PAQR9, IFNGR1, IGFBP1, KRTAP10-10, IL13RA1, KCNA3, KCNA5, TPTE2P6, LHFPL3, KCNJ10, LINC00162, KCNS3, KRTAP5-1, CC2D2B, RPRML, LRRC14B, OR52N2, LAMA4, LIPE, LINC00200, LINC00668, ZNF880, LOC400756, ASB18, LRRD1, LDLRAD2, LYN, MIR127, MIR210, MIR181A1, MIR9-1, MIR98, MIR99B, MIR17HG, MAFG, MAS1, MAX, MLF1, SUGT1P1, RPS26P11, MY010, NDUFB9, NKX3-1, NOS1, NRGN, MIR375, DUOX2, RRP15, NMD3, KLHL5, RPL26L1, EGFL7, PCYT1A, CDC40, UFC1, ERGIC3, SF3B6, PFDN4, PIK3C2G, DDIT4, EGLN1, FAM20A, HEATR3, CEP72, THSD1, PSME2, MIR362, MIR494, MIR520C, MIR516B2, MIR502, MIR508, ERMN, RANBP10, SH3RF1, FBRSL1, RET, EXOC4, TGIF2, RPL3L, BCORL1, CCL5, SDCBP, RRAGC, XYLT2, ACOT6, POPDC3, LINC00535, CCT6P1, TSG1, NA, BLVRA, NARFL, CSMD1, SGSH, NDST4, NA, EFTUD1P1, SIPA1, SLC5A4, PCP4L1, SLC9A2, LYNX1, MIR539, CAPN15, SPAG1, SPRR3, SQLE, SRI, SCARNA2, SCARNA16, TACR1, SNORD66, SNORD84, SNORD91A, SNORD97, MIR552, MIR563, MIR591, TCF19, TNP1, MIR660, SDHAP3, LOC729224, FAHD2CP, SEC14L6, MEIS1-AS3, WNT9A, SNORD113-3, SNORD114-3, SNORD114-9, SNORD114-18, SNORD114-27, SNORD114-30, MIR758, MIR668, ZNF134, PPDPF, OR5H2, CERS4, OGFOD2, LIN28A, NEK11, DCAKD, GPR157, EFHC2, CXXC4, PBX4, CPTP, NPRL3, OR2B2, AKAP17A, PCDH11Y, FAM103A1, TRAPPC9, HYAL3, CASP5, TTTY11, RAB34, NACAP1, SPOP, ZMYND12, LLPH, C22orf23, GTPBP3, SUV420H2, HAT1, SHANK3, KIAA1755, DENR, NPFF, MARCO, JRKL, PAGE1, PABPC4, SYNGAP1, SGPL1, BUD31, HIST1H3F, SYT8, MAP7, ZFAND2A, RGN, VAPB, RCSD1, SLIT2, NCR2, PAGE4, RNF7, MTFR1, CD59, and ATP2C2. In some cases, when the subject is male, the expression levels of two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, or three hundred or more genes (nucleotide or protein biomarkers) for the plurality of genes listed above are measured and detected.

In some cases, when the subject is male, the expression levels of less than 40, 35, 30, 25, 20, or 15 genes (nucleotide or protein biomarkers) selected from the group of genes consisting of SNORD115-37, SNORD115-24, SNORA11B, MIR876, COL4A2-AS2, LOC100130373, LOC100130548, LOC100131496, LINC00240, BCL2L11, MED16, ERVV-2, KRTAP21-3, LOC100289230, LOC100289283, MIR1275, MIR1912, M1R1302-7, MIR1231, MIR1238, MIR1179, MIR1908, MIR1237, MIR2116, CSN1S2BP, MIR3148, MIR4280, MIR3116-1, MIR3129, MIR4275, MIR4326, MIR4254, LINC00673, MIR3926-2, MIR3678, MIR548Z, MIR3657, MIR3935, LOC100505478, LINC01364, C16orf95, LINC00837, MIR3529, MIR4475, MIR4492, MIR4784, MIR4732, MIR4684, MIR4773-2, MIR4429, MIR4442, LINC00507, LOC100996345, CYYR1-AS1, LOC100996635, CDK4, LOC101927053, PDZRN3-AS1, MEOX2-AS1, LINC01186, NA, GBAT2, LINC01214, LOC101928101, LOC101928118, NA, LOC101928195, LOC101928385, ICONS 00029157, LOC101928668, LINC01450, NA, LOC101928823, LOC101928919, LINC01262, LOC101929031, LOC101929159, LOC101929468, LOC101929497, ZNF267, CHERP, IFI44, FUT9, RFPL2, IFI44L, MAPRE2, SLC27A5, CHD1, RABL2B, FSTL1, MRPL3, GLB1L3, GPR182, CHRM5, SMIM12, LRRC37B, SMYD4, TBCB, KLHDC3, RPL39L, CLTB, PRSS30P, PCP2, FITM2, SNTN, TMEM68, OTUD6A, CRP, DYNLL2, CRYGD, NA, TMC8, CSTB, SLC35F3, CCDC140, WDSUB1, SH3D19, TEX28, FEZF1-AS1, ESCO2, ZNF645, CYP51A1, DAB1, ZCCHC12, DNMT3A, DRP2, EGR2, ABCA2, CDY2B, SENP5, EVI2B, ALCAM, ABCD1, F12, FAH, OR5A2, CCDC89, FABP5P3, FGF4, FGF14, DIP2A, ATP11B, NUP210, EXOC6B, ARC, SASH1, GRIP1, SEC61G, SCRIB, POLA2, GABARAPL1, DEFB108B, DEFB113, FTH1P3, CDRT15L2, HEATR9, ALS2CL, ASPM, OR2F1, GATA2, PLA2G2D, GCNT1, AGO1, OR7A5, OR1J2, SNORA70, GNAI3, BCL9L, OTOGL, SLC46A3, EWSAT1, LINC00927, C16orf54, MAMSTR, MCHR1, LOC284825, IGHD2-21, IGHD2-15, EOGT, C8orf31, IGLV10-54, IGLV3-12, SNX24, ARHGAP35, SCG3, PYCR2, SLC39A3, H3F3B, ANXA5, CXXC1, CDR2L, HNF4A, HNRNPK, SNORD56B, KRTAP20-1, KRTAP20-2, LOC339166, ZFP69, LOC340074, KIAA1875, OR2T8, PAQR9, IFNGR1, IGFBP1, KRTAP10-10, IL13RA1, KCNA3, KCNA5, TPTE2P6, LHFPL3, KCNJ10, LINC00162, KCNS3, KRTAP5-1, CC2D2B, RPRML, LRRC14B, OR52N2, LAMA4, LIPE, LINC00200, LINC00668, ZNF880, LOC400756, ASB18, LRRD1, LDLRAD2, LYN, MIR127, MIR210, MIR181A1, MIR9-1, MIR98, MIR99B, MIR17HG, MAFG, MAS1, MAX, MLF1, SUGT1P1, RPS26P11, MY010, NDUFB9, NKX3-1, NOS1, NRGN, MIR375, DUOX2, RRP15, NMD3, KLHL5, RPL26L1, EGFL7, PCYT1A, CDC40, UFC1, ERGIC3, SF3B6, PFDN4, PIK3C2G, DDIT4, EGLN1, FAM20A, HEATR3, CEP72, THSD1, PSME2, MIR362, MIR494, MIR520C, MIR516B2, MIR502, MIR508, ERMN, RANBP10, SH3RF1, FBRSL1, RET, EXOC4, TGIF2, RPL3L, BCORL1, CCL5, SDCBP, RRAGC, XYLT2, ACOT6, POPDC3, LINC00535, CCT6P1, TSG1, NA, BLVRA, NARFL, CSMD1, SGSH, NDST4, NA, EFTUD1P1, SIPA1, SLC5A4, PCP4L1, SLC9A2, LYNX1, MIR539, CAPN15, SPAG1, SPRR3, SQLE, SRI, SCARNA2, SCARNA16, TACR1, SNORD66, SNORD84, SNORD91A, SNORD97, MIR552, MIR563, MIR591, TCF19, TNP1, MIR660, SDHAP3, LOC729224, FAHD2CP, SEC14L6, MEIS1-AS3, WNT9A, SNORD113-3, SNORD114-3, SNORD114-9, SNORD114-18, SNORD114-27, SNORD114-30, MIR758, MIR668, ZNF134, PPDPF, OR5H2, CERS4, OGFOD2, LIN28A, NEK11, DCAKD, GPR157, EFHC2, CXXC4, PBX4, CPTP, NPRL3, OR2B2, AKAP17A, PCDH11Y, FAM103A1, TRAPPC9, HYAL3, CASP5, TTTY11, RAB34, NACAP1, SPOP, ZMYND12, LLPH, C22orf23, GTPBP3, SUV420H2, HAT1, SHANK3, KIAA1755, DENR, NPFF, MARCO, JRKL, PAGE1, PABPC4, SYNGAP1, SGPL1, BUD31, HIST1H3F, SYT8, MAP7, ZFAND2A, RGN, VAPB, RCSD1, SLIT2, NCR2, PAGE4, RNF7, MTFR1, CD59, ATP2C2, ZBTB11-AS1, SNORD115-2, MIR942, MIR708, NFYC-AS1, MIR181A1HG, LOC100133669, FAR2P2, MIR513C, MIR1468, MIR1538, MIR1301, MIR1197, MIR3125, FAM212B-AS1, LOC100506371, LOC100506730, LOC100507419, LOC100507507, MIR4686, MIR378D1, MIR3689F, MIR4756, MIR4499, MIR550A3, MIR4425, MIR4691, HTT-AS, CERS6-AS1, RNU6-81P, FGF14-AS1, SLC9A9-AS1, DHRS9, LOC101927314, LOC101927483, LINC01280, LINC01488, LOC101928399, LOC101928509, LOC101928579, LOC101928668, LOC101928880, LOC101929236, LOC101929284, LOC101929432, LOC101929636, PSMD14, GPA33, CDO1, RGS14, GMEB1, MAPRE2, C9orf9, RABL2A, BIRC8, KLHL2, GPN1, CKM, RAB3C, OR52M1, CLK3, JDP2, SNX20, SHE, DMBX1, EDARADD, C20orf144, COMP, GLYCTK, UBLCP1, APOOL, RNF113B, CST1, CREB3L4, CTSL, NCBP2-AS2, MGC32805, CRYGN, TMEM30B, HBEGF, DUSPS, DVL2, E4F1, FLJ23867, ZNF385C, EPS8, EVPL, EXTL1, EZH2, FOXK1, DKFZP58611420, DIP2C, ATG2A, WDR43, SCFD1, SIN3B, FMR1, USP22, SZT2, CRTC1, MED13L, DEFB110, PROSER2, C19orf26, GEMIN5, SYF2, RWDD3, GARS, INTS1, GBP2, MYEOV, SNORD14A, B3GAT1, ZNF330, PCDH11XGP9, LOC284009, C17orf89, EMC10, WDR62, OR2V2, HOOK2, RPA4, GYPE, HBA2, HBB, HELLS, FOXA2, HNRNPF, HPN, HPRT1, ERAS, HSD17B1, NRBP2, IDI1, FIGLA, RTN4RL2, AQP1, NANOGNB, PDX1, C9orf50, SLC27A1, TRIM74, KIR2DL3, KRTAP12-3, C10orf99, MFSD2B, DKFZp667F0711, SOHLH1, MIR188, MIR26B, MIR296, MAN2A2, MGST3, MLH1, LOC440910, SMIM4, MYBPH, NASP, NDUFA1, NDUFS1, NFIL3, RNASE12, MIR380, OXTR, TXNDC11, RPS27L, SBDS, POMP, UBE2J1, CWC15, ATP6V1H, RBMX2, SERPINB8, TLR9, FAM193B, NUTM2F, DNAJC25, EPN3, KANSL3, VIMP, ZC3H15, EIF2AK2, PSMA2, PSMA4, MIR412, PRR12, ZNF492, SQRDL, RPL28, MIR487A, RPS6KA3, SCN4B, FAM204A, IRF2BPL, ZNF862, GZF 1, ATG3, GGT8P, EBF2, SI, SKI, FOXD4L6, SPAG11A, SLN, MIR376A2, FOXL2, TCF3, MIR550A2, DNAJC7, LOC727710, MRS2P2, LOC729739, TMEM258, NELFA, ZNF41, SNORD113-5, SNORD113-8, MIR769, MIR766, CLMP, GGNBP2, BRD3, SSPN, TUBB1, COIL, SF3A2, NRIP1, PARP9, HSDL2, EIF1AD, BRMS1L, SYVN1, TUBB6, USMGS, POMGNT2, MA STL, MIR22HG, IFITM1, KIAA1656, LRRCC1, KRT38, SUCLA2, DPM1, CDC123, DDX18, CHRNA6, KLHL6, BRSK2, ZNF622, PAGE5, PDLIM1, BTF3L4, DYRK1B, RAB11B, GRAP2, MUC16, CGB8, MED21, EP STI1, CYTIP, NCOR2, and TMEM63A.

In some embodiments, when the subject is male, the expression levels of less than 40, 35, 30, 20, or 15 genes (nucleotide or protein biomarker) detected using the plurality of probes and/or mRNA targets listed in Table 4 are measured and detected. In some cases, the plurality of genes detected using probes and/or mRNA targets selected from Table 4 are selected from the group of genes in Table 21. In some embodiments, when the subject is male, the expression levels of less than 40, 35, 25, 20, or 15 genes (nucleotide or protein biomarker) from said plurality of exon regions of genes (nucleotide or protein sequences) listed in Table 6 are measured and detected. In some cases, the plurality of exon regions of genes selected from Table 6 are selected from the group of genes in Table 22.

In some cases, the plurality of exon regions of genes selected from Table 6 comprise at least one gene selected from the group consisting of Gene Number 4155 (PLCH2), Gene Number 4166 (VPS13D), Gene Number 4167 (PDPN), Gene Number 4169 (DNAJC16), Gene Number 4181 (LDLRAP1), Gene Number 4184 (RPS6KA1), Gene Number 4203 (MFSD2A), Gene Number 4241, Gene Number 4462 (ZMYND12), Gene Number 4562 (TNFSF18), Gene Number 5518, Gene Number 5726 (LMBR1L). In some cases, the plurality of exon regions of genes selected from Table 6 comprise at least two genes selected from the group consisting of Gene Number 4155 (PLCH2), Gene Number 4166 (VPS13D), Gene Number 4167 (PDPN), Gene Number 4169 (DNAJC16), Gene Number 4181 (LDLRAP1), Gene Number 4184 (RPS6KA1), Gene Number 4203 (MFSD2A), Gene Number 4241, Gene Number 4462 (ZMYND12), Gene Number 4562 (TNFSF18), Gene Number 5518, Gene Number 5726 (LMBR1L). In some cases, the plurality of exon regions of genes selected from Table 6 comprise at least three genes selected from the group consisting of Gene Number 4155 (PLCH2), Gene Number 4166 (VPS13D), Gene Number 4167 (PDPN), Gene Number 4169 (DNAJC16), Gene Number 4181 (LDLRAP1), Gene Number 4184 (RPS6KA1), Gene Number 4203 (MFSD2A), Gene Number 4241, Gene Number 4462 (ZMYND12), Gene Number 4562 (TNFSF18), Gene Number 5518, Gene Number 5726 (LMBR1L). In some cases, the plurality of exon regions of genes selected from Table 6 comprise at least four genes selected from the group consisting of Gene Number 4155 (PLCH2), Gene Number 4166 (VPS13D), Gene Number 4167 (PDPN), Gene Number 4169 (DNAJC16), Gene Number 4181 (LDLRAP1), Gene Number 4184 (RPS6KA1), Gene Number 4203 (MFSD2A), Gene Number 4241, Gene Number 4462 (ZMYND12), Gene Number 4562 (TNFSF18), Gene Number 5518, Gene Number 5726 (LMBR1L). In some cases, the plurality of exon regions of genes selected from Table 6 comprise at least five genes selected from the group consisting of Gene Number 4155 (PLCH2), Gene Number 4166 (VPS13D), Gene Number 4167 (PDPN), Gene Number 4169 (DNAJC16), Gene Number 4181 (LDLRAP1), Gene Number 4184 (RPS6KA1), Gene Number 4203 (MFSD2A), Gene Number 4241, Gene Number 4462 (ZMYND12), Gene Number 4562 (TNFSF18), Gene Number 5518, Gene Number 5726 (LMBR1L). In some cases, the plurality of exon regions of genes selected from Table 6 comprise at least six genes selected from the group consisting of Gene Number 4155 (PLCH2), Gene Number 4166 (VPS13D), Gene Number 4167 (PDPN), Gene Number 4169 (DNAJC16), Gene Number 4181 (LDLRAP1), Gene Number 4184 (RPS6KA1), Gene Number 4203 (MFSD2A), Gene Number 4241, Gene Number 4462 (ZMYND12), Gene Number 4562 (TNFSF18), Gene Number 5518, Gene Number 5726 (LMBR1L). In some cases, the plurality of exon regions of genes selected from Table 6 comprise Gene Number 4155 (PLCH2), Gene Number 4166 (VPS13D), Gene Number 4167 (PDPN), Gene Number 4169 (DNAJC16), Gene Number 4181 (LDLRAP1), Gene Number 4184 (RPS6KA1), Gene Number 4203 (MFSD2A), Gene Number 4241, Gene Number 4462 (ZMYND12), Gene Number 4562 (TNFSF18), Gene Number 5518, Gene Number 5726 (LMBR1L). In some cases, the plurality of exon regions of genes selected from Table 6 consist of Gene Number 4155 (PLCH2), Gene Number 4166 (VPS13D), Gene Number 4167 (PDPN), Gene Number 4169 (DNAJC16), Gene Number 4181 (LDLRAP1), Gene Number 4184 (RPS6KA1), Gene Number 4203 (MFSD2A), Gene Number 4241, Gene Number 4462 (ZMYND12), Gene Number 4562 (TNFSF18), Gene Number 5518, Gene Number 5726 (LMBR1L).

In some cases, the plurality of exon regions of genes selected from Table 6 comprise at least one gene selected from the group consisting of Gene Number 4151 (LOC284628), Gene Number 4216 (DMAP1), Gene Number 4258 (LOC100996635), Gene Number 4444, Gene Number 5586, Gene Number 5590 (TRMTS), Gene Number 5879 (IRX6), Gene Number 6181 (FBXL20), Gene Number 6214 (MEP1B), Gene Number 6810, Gene Number 7536 (FGFR3), and Gene Number 8887 (DNAJC25). In some cases, the plurality of genes of exon regions selected from Table 6 comprise at least two genes selected from the group consisting of Gene Number 4151 (LOC284628), Gene Number 4216 (DMAP1), Gene Number 4258 (LOC100996635), Gene Number 4444, Gene Number 5586, Gene Number 5590 (TRMTS), Gene Number 5879 (IRX6), Gene Number 6181 (FBXL20), Gene Number 6214 (MEP1B), Gene Number 6810, Gene Number 7536 (FGFR3), and Gene Number 8887 (DNAJC25). In some cases, the plurality of exon regions of genes selected from Table 6 comprise at least three genes selected from the group consisting of Gene Number 4151 (LOC284628), Gene Number 4216 (DMAP1), Gene Number 4258 (LOC100996635), Gene Number 4444, Gene Number 5586, Gene Number 5590 (TRMTS), Gene Number 5879 (IRX6), Gene Number 6181 (FBXL20), Gene Number 6214 (MEP1B), Gene Number 6810, Gene Number 7536 (FGFR3), and Gene Number 8887 (DNAJC25). In some cases, the plurality of exon regions of genes selected from Table 6 comprise at least four genes selected from the group consisting of Gene Number 4151 (LOC284628), Gene Number 4216 (DMAP1), Gene Number 4258 (LOC100996635), Gene Number 4444, Gene Number 5586, Gene Number 5590 (TRMTS), Gene Number 5879 (IRX6), Gene Number 6181 (FBXL20), Gene Number 6214 (MEP1B), Gene Number 6810, Gene Number 7536 (FGFR3), and Gene Number 8887 (DNAJC25). In some cases, the plurality of exon regions of genes selected from Table 6 comprise at least five genes selected from the group consisting of Gene Number 4151 (LOC284628), Gene Number 4216 (DMAP1), Gene Number 4258 (LOC100996635), Gene Number 4444, Gene Number 5586, Gene Number 5590 (TRMTS), Gene Number 5879 (IRX6), Gene Number 6181 (FBXL20), Gene Number 6214 (MEP1B), Gene Number 6810, Gene Number 7536 (FGFR3), and Gene Number 8887 (DNAJC25). In some cases, the plurality of exon regions of genes selected from Table 6 comprise at least six genes selected from the group consisting of Gene Number 4151 (LOC284628), Gene Number 4216 (DMAP1), Gene Number 4258 (LOC100996635), Gene Number 4444, Gene Number 5586, Gene Number 5590 (TRMTS), Gene Number 5879 (IRX6), Gene Number 6181 (FBXL20), Gene Number 6214 (MEP1B), Gene Number 6810, Gene Number 7536 (FGFR3), and Gene Number 8887 (DNAJC25). In some cases, the plurality of exon regions of genes selected from Table 6 comprise Gene Number 4151 (LOC284628), Gene Number 4216 (DMAP1), Gene Number 4258 (LOC100996635), Gene Number 4444, Gene Number 5586, Gene Number 5590 (TRMTS), Gene Number 5879 (IRX6), Gene Number 6181 (FBXL20), Gene Number 6214 (MEP1B), Gene Number 6810, Gene Number 7536 (FGFR3), and Gene Number 8887 (DNAJC25). In some cases, the plurality of exon regions of genes selected from Table 6 consist of Gene Number 4151 (LOC284628), Gene Number 4216 (DMAP1), Gene Number 4258 (LOC100996635), Gene Number 4444, Gene Number 5586, Gene Number 5590 (TRMTS), Gene Number 5879 (IRX6), Gene Number 6181 (FBXL20), Gene Number 6214 (MEP1B), Gene Number 6810, Gene Number 7536 (FGFR3), and Gene Number 8887 (DNAJC25).

In some cases, the plurality of exon regions of genes selected from Table 6 comprise at least one gene selected from the group consisting of Gene Number 4164 (TMEM201), Gene Number 4166 (VPS13D), Gene Number 4337 (IPO9), Gene Number 4344 (LAX1), Gene Number 4350 (MFSD4), Gene Number 4356 (DTL), Gene Number 4578 (MIR181A1HG), Gene Number 4709 (GBF1), Gene Number 5478 (PNN), Gene Number 5518, and Gene Number 7345 (OTTHUMG00000162679). In some cases, the plurality of exon regions of genes selected from Table 6 comprise at least two genes selected from the group consisting of Gene Number 4164 (TMEM201), Gene Number 4166 (VPS13D), Gene Number 4337 (IPO9), Gene Number 4344 (LAX1), Gene Number 4350 (MFSD4), Gene Number 4356 (DTL), Gene Number 4578 (MIR181A1HG), Gene Number 4709 (GBF1), Gene Number 5478 (PNN), Gene Number 5518, and Gene Number 7345 (OTTHUMG00000162679). In some cases, the plurality of exon regions of genes selected from Table 6 comprise at least three genes selected from the group consisting of Gene Number 4164 (TMEM201), Gene Number 4166 (VPS13D), Gene Number 4337 (IPO9), Gene Number 4344 (LAX1), Gene Number 4350 (MFSD4), Gene Number 4356 (DTL), Gene Number 4578 (MIR181A1HG), Gene Number 4709 (GBF1), Gene Number 5478 (PNN), Gene Number 5518, and Gene Number 7345 (OTTHUMG00000162679). In some cases, the plurality of exon regions of genes selected from Table 6 comprise at least four genes selected from the group consisting of Gene Number 4164 (TMEM201), Gene Number 4166 (VPS13D), Gene Number 4337 (IPO9), Gene Number 4344 (LAX1), Gene Number 4350 (MFSD4), Gene Number 4356 (DTL), Gene Number 4578 (MIR181A1HG), Gene Number 4709 (GBF1), Gene Number 5478 (PNN), Gene Number 5518, and Gene Number 7345 (OTTHUMG00000162679). In some cases, the plurality of exon regions of genes selected from Table 6 comprise at least five genes selected from the group consisting of Gene Number 4164 (TMEM201), Gene Number 4166 (VPS13D), Gene Number 4337 (IPO9), Gene Number 4344 (LAX1), Gene Number 4350 (MFSD4), Gene Number 4356 (DTL), Gene Number 4578 (MIR181A1HG), Gene Number 4709 (GBF1), Gene Number 5478 (PNN), Gene Number 5518, and Gene Number 7345 (OTTHUMG00000162679). In some cases, the plurality of exon regions of genes selected from Table 6 comprise at least six genes selected from the group consisting of Gene Number 4164 (TMEM201), Gene Number 4166 (VPS13D), Gene Number 4337 (IPO9), Gene Number 4344 (LAX1), Gene Number 4350 (MFSD4), Gene Number 4356 (DTL), Gene Number 4578 (MIR181A1HG), Gene Number 4709 (GBF1), Gene Number 5478 (PNN), Gene Number 5518, and Gene Number 7345 (OTTHUMG00000162679). In some cases, the plurality of exon regions of genes selected from Table 6 comprise Gene Number 4164 (TMEM201), Gene Number 4166 (VPS13D), Gene Number 4337 (IPO9), Gene Number 4344 (LAX1), Gene Number 4350 (MFSD4), Gene Number 4356 (DTL), Gene Number 4578 (MIR181A1HG), Gene Number 4709 (GBF1), Gene Number 5478 (PNN), Gene Number 5518, and Gene Number 7345 (OTTHUMG00000162679). In some cases, the plurality of exon regions of genes selected from Table 6 consist of Gene Number 4164 (TMEM201), Gene Number 4166 (VPS13D), Gene Number 4337 (IPO9), Gene Number 4344 (LAX1), Gene Number 4350 (MFSD4), Gene Number 4356 (DTL), Gene Number 4578 (MIR181A1HG), Gene Number 4709 (GBF1), Gene Number 5478 (PNN), Gene Number 5518, and Gene Number 7345 (OTTHUMG00000162679).

In some cases, the plurality of exon regions of genes selected from Table 6 comprise at least one gene selected from the group consisting of Gene Number 4257 (RWDD3), Gene Number 4628 (ATP5C1), Gene Number 5514 (TDP1), Gene Number 5681 (FAM81A), Gene Number 5696 (NR2E3), Gene Number 5713 (SEMA4B), Gene Number 5764 (MGC15885), Gene Number 6810, Gene Number 6876, Gene Number 7053 (IL10RB), and Gene Number 7155 (POLR2F). In some cases, the plurality of exon regions of genes selected from Table 6 comprise at least two genes selected from the group consisting of Gene Number 4257 (RWDD3), Gene Number 4628 (ATP5C1), Gene Number 5514 (TDP1), Gene Number 5681 (FAM81A), Gene Number 5696 (NR2E3), Gene Number 5713 (SEMA4B), Gene Number 5764 (MGC15885), Gene Number 6810, Gene Number 6876, Gene Number 7053 (IL10RB), and Gene Number 7155 (POLR2F). In some cases, the plurality of exon regions of genes selected from Table 6 comprise at least three genes selected from the group consisting of Gene Number 4257 (RWDD3), Gene Number 4628 (ATP5C1), Gene Number 5514 (TDP1), Gene Number 5681 (FAM81A), Gene Number 5696 (NR2E3), Gene Number 5713 (SEMA4B), Gene Number 5764 (MGC15885), Gene Number 6810, Gene Number 6876, Gene Number 7053 (IL10RB), and Gene Number 7155 (POLR2F). In some cases, the plurality of exon regions of genes selected from Table 6 comprise at least four genes selected from the group consisting of Gene Number 4257 (RWDD3), Gene Number 4628 (ATP5C1), Gene Number 5514 (TDP1), Gene Number 5681 (FAM81A), Gene Number 5696 (NR2E3), Gene Number 5713 (SEMA4B), Gene Number 5764 (MGC15885), Gene Number 6810, Gene Number 6876, Gene Number 7053 (IL10RB), and Gene Number 7155 (POLR2F). In some cases, the plurality of exon regions of genes selected from Table 6 comprise at least five genes selected from the group consisting of Gene Number 4257 (RWDD3), Gene Number 4628 (ATP5C1), Gene Number 5514 (TDP1), Gene Number 5681 (FAM81A), Gene Number 5696 (NR2E3), Gene Number 5713 (SEMA4B), Gene Number 5764 (MGC15885), Gene Number 6810, Gene Number 6876, Gene Number 7053 (IL10RB), and Gene Number 7155 (POLR2F). In some cases, the plurality of exon regions of genes selected from Table 6 comprise at least six genes selected from the group consisting of Gene Number 4257 (RWDD3), Gene Number 4628 (ATP5C1), Gene Number 5514 (TDP1), Gene Number 5681 (FAM81A), Gene Number 5696 (NR2E3), Gene Number 5713 (SEMA4B), Gene Number 5764 (MGC15885), Gene Number 6810, Gene Number 6876, Gene Number 7053 (IL10RB), and Gene Number 7155 (POLR2F). In some cases, the plurality of exon regions of genes selected from Table 6 comprise Gene Number 4257 (RWDD3), Gene Number 4628 (ATP5C1), Gene Number 5514 (TDP1), Gene Number 5681 (FAM81A), Gene Number 5696 (NR2E3), Gene Number 5713 (SEMA4B), Gene Number 5764 (MGC15885), Gene Number 6810, Gene Number 6876, Gene Number 7053 (IL10RB), and Gene Number 7155 (POLR2F). In some cases, the plurality of exon regions of genes selected from Table 6 consist of Gene Number 4257 (RWDD3), Gene Number 4628 (ATP5C1), Gene Number 5514 (TDP1), Gene Number 5681 (FAM81A), Gene Number 5696 (NR2E3), Gene Number 5713 (SEMA4B), Gene Number 5764 (MGC15885), Gene Number 6810, Gene Number 6876, Gene Number 7053 (IL10RB), and Gene Number 7155 (POLR2F).

In some cases, the plurality of exon regions of genes selected from Table 6 comprise at least one gene selected from the group consisting of Gene Number 4151 (PLEKHN1), Gene Number 4337 (IPO9), Gene Number 4350 (MFSD4), Gene Number 4381 (OR2L13), Gene Number 4402 (FLJ42875), Gene Number 4460 (LOC100130557), Gene Number 4511 (CASQ2), Gene Number 4915 (HEPHL1), and Gene Number 7275 (SLC22A14). In some cases, the plurality of exon regions of genes selected from Table 6 comprise at least two genes selected from the group consisting of Gene Number 4151 (PLEKHN1), Gene Number 4337 (IPO9), Gene Number 4350 (MFSD4), Gene Number 4381 (OR2L13), Gene Number 4402 (FLJ42875), Gene Number 4460 (LOC100130557), Gene Number 4511 (CASQ2), Gene Number 4915 (HEPHL1), and Gene Number 7275 (SLC22A14). In some cases, the plurality of exon regions of genes selected from Table 6 comprise at least three genes selected from the group consisting of Gene Number 4151 (PLEKHN1), Gene Number 4337 (IPO9), Gene Number 4350 (MFSD4), Gene Number 4381 (OR2L13), Gene Number 4402 (FLJ42875), Gene Number 4460 (LOC100130557), Gene Number 4511 (CASQ2), Gene Number 4915 (HEPHL1), and Gene Number 7275 (SLC22A14). In some cases, the plurality of exon regions of genes selected from Table 6 comprise at least four genes selected from the group consisting of Gene Number 4151 (PLEKHN1), Gene Number 4337 (IPO9), Gene Number 4350 (MFSD4), Gene Number 4381 (OR2L13), Gene Number 4402 (FLJ42875), Gene Number 4460 (LOC100130557), Gene Number 4511 (CASQ2), Gene Number 4915 (HEPHL1), and Gene Number 7275 (SLC22A14). In some cases, the plurality of genes selected from Table 6 comprise at least five genes selected from the group consisting of exon regions of Gene Number 4151 (PLEKHN1), Gene Number 4337 (IPO9), Gene Number 4350 (MFSD4), Gene Number 4381 (OR2L13), Gene Number 4402 (FLJ42875), Gene Number 4460 (LOC100130557), Gene Number 4511 (CASQ2), Gene Number 4915 (HEPHL1), and Gene Number 7275 (SLC22A14). In some cases, the plurality of exon regions of genes selected from Table 6 comprise at least six genes selected from the group consisting of Gene Number 4151 (PLEKHN1), Gene Number 4337 (IPO9), Gene Number 4350 (MFSD4), Gene Number 4381 (OR2L13), Gene Number 4402 (FLJ42875), Gene Number 4460 (LOC100130557), Gene Number 4511 (CASQ2), Gene Number 4915 (HEPHL1), and Gene Number 7275 (SLC22A14). In some cases, the plurality of exon regions of genes selected from Table 6 comprise Gene Number 4151 (PLEKHN1), Gene Number 4337 (IPO9), Gene Number 4350 (MFSD4), Gene Number 4381 (OR2L13), Gene Number 4402 (FLJ42875), Gene Number 4460 (LOC100130557), Gene Number 4511 (CASQ2), Gene Number 4915 (HEPHL1), and Gene Number 7275 (SLC22A14). In some cases, the plurality of exon regions of genes selected from Table 6 consist of Gene Number 4151 (PLEKHN1), Gene Number 4337 (IPO9), Gene Number 4350 (MFSD4), Gene Number 4381 (OR2L13), Gene Number 4402 (FLJ42875), Gene Number 4460 (LOC100130557), Gene Number 4511 (CASQ2), Gene Number 4915 (HEPHL1), and Gene Number 7275 (SLC22A14).

In some cases, the plurality of exon regions of genes selected from Table 6 comprise at least one gene selected from the group consisting of Gene Number 4167 (PDPN), Gene Number 4173 (PLA2G5), Gene Number 4196, Gene Number 4176 (EPHA8), Gene Number 4352 (PFKFB2), Gene Number 4837, Gene Number 5491 (PPM1A), Gene Number 6266 (DTNA), Gene Number 6813 (FAM178B), and Gene Number 7393. In some cases, the plurality of exon regions of genes selected from Table 6 comprise at least two genes selected from the group consisting of Gene Number 4167 (PDPN), Gene Number 4173 (PLA2G5), Gene Number 4196, Gene Number 4176 (EPHA8), Gene Number 4352 (PFKFB2), Gene Number 4837, Gene Number 5491 (PPM1A), Gene Number 6266 (DTNA), Gene Number 6813 (FAM178B), and Gene Number 7393. In some cases, the plurality of exon regions of genes selected from Table 6 comprise at least three genes selected from the group consisting of Gene Number 4167 (PDPN), Gene Number 4173 (PLA2G5), Gene Number 4196, Gene Number 4176 (EPHA8), Gene Number 4352 (PFKFB2), Gene Number 4837, Gene Number 5491 (PPM1A), Gene Number 6266 (DTNA), Gene Number 6813 (FAM178B), and Gene Number 7393. In some cases, the plurality of exon regions of genes selected from Table 6 comprise at least four genes selected from the group consisting of Gene Number 4167 (PDPN), Gene Number 4173 (PLA2G5), Gene Number 4196, Gene Number 4176 (EPHA8), Gene Number 4352 (PFKFB2), Gene Number 4837, Gene Number 5491 (PPM1A), Gene Number 6266 (DTNA), Gene Number 6813 (FAM178B), and Gene Number 7393. In some cases, the plurality of exon regions of genes selected from Table 6 comprise at least five genes selected from the group consisting of Gene Number 4167 (PDPN), Gene Number 4173 (PLA2G5), Gene Number 4196, Gene Number 4176 (EPHA8), Gene Number 4352 (PFKFB2), Gene Number 4837, Gene Number 5491 (PPM1A), Gene Number 6266 (DTNA), Gene Number 6813 (FAM178B), and Gene Number 7393. In some cases, the plurality of exon regions of genes selected from Table 6 comprise at least six genes selected from the group consisting of Gene Number 4167 (PDPN), Gene Number 4173 (PLA2G5), Gene Number 4196, Gene Number 4176 (EPHA8), Gene Number 4352 (PFKFB2), Gene Number 4837, Gene Number 5491 (PPM1A), Gene Number 6266 (DTNA), Gene Number 6813 (FAM178B), and Gene Number 7393. In some cases, the plurality of exon regions of genes selected from Table 6 comprise Gene Number 4167 (PDPN), Gene Number 4173 (PLA2G5), Gene Number 4196, Gene Number 4176 (EPHA8), Gene Number 4352 (PFKFB2), Gene Number 4837, Gene Number 5491 (PPM1A), Gene Number 6266 (DTNA), Gene Number 6813 (FAM178B), and Gene Number 7393. In some cases, the plurality of exon regions of genes selected from Table 6 consist of Gene Number 4167 (PDPN), Gene Number 4173 (PLA2G5), Gene Number 4196, Gene Number 4176 (EPHA8), Gene Number 4352 (PFKFB2), Gene Number 4837, Gene Number 5491 (PPM1A), Gene Number 6266 (DTNA), Gene Number 6813 (FAM178B), and Gene Number 7393.

In some cases, the plurality of exon regions of genes selected from Table 6 comprise at least one gene selected from the group consisting of Gene Number 4145 (PLEKHN1), Gene Number 4166 (VPS13D), Gene Number 4590 (PLXNA2), Gene Number 4952 (ST14), Gene Number 5042 (LTBP3), Gene Number 5181 (ACSS3), Gene Number 5209 (ATP2A2), Gene Number 6714 (BCS1L), Gene Number 6809, Gene Number 7007 (CPNE1), Gene Number 7354 (COL6A6), Gene Number 7458 (LINC00971), and Gene Number 7474 (HGD). In some cases, the plurality of genes of exon regions selected from Table 6 comprise at least two genes selected from the group consisting of Gene Number 4145 (PLEKHN1), Gene Number 4166 (VPS13D), Gene Number 4590 (PLXNA2), Gene Number 4952 (ST14), Gene Number 5042 (LTBP3), Gene Number 5181 (ACSS3), Gene Number 5209 (ATP2A2), Gene Number 6714 (BCS1L), Gene Number 6809, Gene Number 7007 (CPNE1), Gene Number 7354 (COL6A6), Gene Number 7458 (LINC00971), and Gene Number 7474 (HGD). In some cases, the plurality of exon regions of genes selected from Table 6 comprise at least three genes selected from the group consisting of Gene Number 4145 (PLEKHN1), Gene Number 4166 (VPS13D), Gene Number 4590 (PLXNA2), Gene Number 4952 (ST14), Gene Number 5042 (LTBP3), Gene Number 5181 (ACSS3), Gene Number 5209 (ATP2A2), Gene Number 6714 (BCS1L), Gene Number 6809, Gene Number 7007 (CPNE1), Gene Number 7354 (COL6A6), Gene Number 7458 (LINC00971), and Gene Number 7474 (HGD). In some cases, the plurality of exon regions of genes selected from Table 6 comprise at least four genes selected from the group consisting of Gene Number 4145 (PLEKHN1), Gene Number 4166 (VPS13D), Gene Number 4590 (PLXNA2), Gene Number 4952 (ST14), Gene Number 5042 (LTBP3), Gene Number 5181 (ACSS3), Gene Number 5209 (ATP2A2), Gene Number 6714 (BCS1L), Gene Number 6809, Gene Number 7007 (CPNE1), Gene Number 7354 (COL6A6), Gene Number 7458 (LINC00971), and Gene Number 7474 (HGD). In some cases, the plurality of exon regions of genes selected from Table 6 comprise at least five genes selected from the group consisting of Gene Number 4145 (PLEKHN1), Gene Number 4166 (VPS13D), Gene Number 4590 (PLXNA2), Gene Number 4952 (ST14), Gene Number 5042 (LTBP3), Gene Number 5181 (ACSS3), Gene Number 5209 (ATP2A2), Gene Number 6714 (BCS1L), Gene Number 6809, Gene Number 7007 (CPNE1), Gene Number 7354 (COL6A6), Gene Number 7458 (LINC00971), and Gene Number 7474 (HGD). In some cases, the plurality of exon regions of genes selected from Table 6 comprise at least six genes selected from the group consisting of Gene Number 4145 (PLEKHN1), Gene Number 4166 (VPS13D), Gene Number 4590 (PLXNA2), Gene Number 4952 (ST14), Gene Number 5042 (LTBP3), Gene Number 5181 (ACSS3), Gene Number 5209 (ATP2A2), Gene Number 6714 (BCS1L), Gene Number 6809, Gene Number 7007 (CPNE1), Gene Number 7354 (COL6A6), Gene Number 7458 (LINC00971), and Gene Number 7474 (HGD). In some cases, the plurality of exon regions of genes selected from Table 6 comprise Gene Number 4145 (PLEKHN1), Gene Number 4166 (VPS13D), Gene Number 4590 (PLXNA2), Gene Number 4952 (ST14), Gene Number 5042 (LTBP3), Gene Number 5181 (ACSS3), Gene Number 5209 (ATP2A2), Gene Number 6714 (BCS1L), Gene Number 6809, Gene Number 7007 (CPNE1), Gene Number 7354 (COL6A6), Gene Number 7458 (LINC00971), and Gene Number 7474 (HGD). In some cases, the plurality of genes selected from Table 6 consist of Gene Number 4145 (PLEKHN1), Gene Number 4166 (VPS13D), Gene Number 4590 (PLXNA2), Gene Number 4952 (ST14), Gene Number 5042 (LTBP3), Gene Number 5181 (ACSS3), Gene Number 5209 (ATP2A2), Gene Number 6714 (BCS1L), Gene Number 6809, Gene Number 7007 (CPNE1), Gene Number 7354 (COL6A6), Gene Number 7458 (LINC00971), and Gene Number 7474 (HGD).

Further, the expression levels may be compared to control ranges disclosed here to generate an output/report indicative of the subject's having or risk of having a condition comprising perfusion shortage with an accuracy greater than about 99%. In some embodiments, the accuracy is about 98.5%, 98%, 97.5%, 97%, 96.5%, 96%, 95.5%, 95%, 94.5%, 94%, 93.5%, 93%, 92.5%, 92%, or 91.5%.

TABLE 21

| Code | | |
|---|---|---|
| PSC | biomarker nucleotide seq Probe | biomarker nucleotide seq target mRNA |
| 1673 | GACCGGGTGTTGGTGCGGCTATTAC | CTGGCCCACAACCACGCCGATAATG |
| 6351 | AGAAGGAGCCCGACCAGGTTCTACC | TCTTCCTCGGGCTGGTCCAAGATGG |
| 6767 | GTGGTGGTGCCCACTGTGGTCCTAC | CACCACCACGGGTGACACCAGGATG |
| 6774 | CTGTGTGTGCCCTTCACAAGAGGTT | GACACACACGGGAAGTGTTCTCCAA |
| 9512 | CTCGGCGCCCAGCTGCGTGCGTGCA | GAGCCGCGGGTCGACGCACGCACGT |
| 11081 | GGAGACTCTCCCAAAGGAACCATAG | CCTCTGAGAGGGTTTCCTTGGTATC |
| 15750 | CTCGAGTCTCCCAGAGGTCCATTGT | GAGCTCAGAGGGTCTCCAGGTAACA |
| 15852 | CTTTATCCCAGAAATGGTGACCGGT | GAAATAGGGTCTTTACCACTGGCCA |
| 16655 | ACTATCTACTCCCAGTAGTGACGCG | TGATAGATGAGGGTCATCACTGCGC |
| 18080 | GTCTGCCGACCCACCGTTTATGTTT | CAGACGGCTGGGTGGCAAATACAAA |
| 19099 | GGACACACACCCACGTGTAAACACA | CCTGTGTGTGGGTGCACATTTGTGT |
| 19994 | AAGACCTACCCACGTCCGACATGGT | TTCTGGATGGGTGCAGGCTGTACCA |
| 21717 | GTACGCTTAGGCCAGTCTTGACCAG | CATGCGAATCCGGTCAGAACTGGTC |
| 27227 | GAAGGTCTTCCCGTTGTTCGCTCCA | CTTCCAGAAGGGCAACAAGCGAGGT |
| 27604 | GAACTTCCGTCCCTGCACTGAAAGG | CTTGAAGGCAGGGACGTGACTTTCC |
| 29415 | CCGAATCTCCCTCTGTGTAGAGTCT | GGCTTAGAGGGAGACACATCTCAGA |
| 36768 | TTACTAGACGTTCCAGAGTCAGGGG | AATGATCTGCAAGGTCTCAGTCCCC |
| 39687 | GATCCTTTCCACTCGCGCTAGAGCG | CTAGGAAAGGTGAGCGCGATCTCGC |
| 41366 | CGCCGCAGTCCACCGCCGACGGCGT | GCGGCGTCAGGTGGCGGCTGCCGCA |
| 42955 | CTCTCCGTGTCCAAAGTTCTGTGTA | GAGAGGCACAGGTTTCAAGACACAT |
| 49089 | AGTCCAGACCCTTCGTGACTGGTCG | TCAGGTCTGGGAAGCACTGACCAGC |
| 53130 | ATAAGAGACCCTCACTGAGTCACGG | TATTCTCTGGGAGTGACTCAGTGCC |
| 53947 | AGGTCCGACCCTCTCACGAGTGGAT | TCCAGGCTGGGAGAGTGCTCACCTA |
| 55338 | GTACACACAGGCCTTCACACACGGG | CATGTGTGTCCGGAAGTGTGTGCCC |
| 58129 | AGAGTTTCCCTAAAGTTCTACGACA | TCTCAAAGGGATTTCAAGATGCTGT |
| 58782 | GTCACGCCTCGCCTCAGCCAAGACC | CAGTGCGGAGCGGAGTCGGTTCTGG |
| 59746 | GGACCGGGCCTACCACGACCGAGGT | CCTGGCCCGGATGGTGCTGGCTCCA |
| 60909 | ACGACCAGCGCCTAGACGACATGGA | TGCTGGTCGCGGATCTGCTGTACCT |
| 64432 | ACTCCGTCCTCCTTTCTGGATGAAG | TGAGGCAGGAGGAAAGACCTACTTC |
| 65832 | GTTACGGACCTCCTCACATCGGCAG | CAATGCCTGGAGGAGTGTAGCCGTC |
| 72889 | GGTTCGGTGTCCTCGAATCGTGTCA | CCAAGCCACAGGAGCTTAGCACAGT |
| 74978 | GGTCTCCACGTCCTCGTTCAAGTGT | CCAGAGGTGCAGGAGCAAGTTCACA |
| 75770 | CTTCTTGTCCTAACCGGTCTACGCA | GAAGAACAGGATTGGCCAGATGCGT |
| 76500 | TTTAAGCTCCTACCGGATAGAGTGT | AAATTCGAGGATGGCCTATCTCACA |
| 89931 | AATCCTACACCACTCCTCTTCGACG | TTAGGATGTGGTGAGGAGAAGCTGC |
| 100396 | AGACCACACACCTTTGACTGCCACC | TCTGGTGTGTGGAAACTGACGGTGG |
| 125863 | AATCCGGTCCGACATCGACCAGGAG | TTAGGCCAGGCTGTAGCTGGTCCTC |
| 128031 | CCGCACGACTCCGACTTTTACCGTA | GGCGTGCTGAGGCTGAAAATGGCAT |
| 128748 | AAGAGTTCCCGTTATTTCAATCCGT | TTCTCAAGGGCAATAAAGTTAGGCA |
| 128806 | CGATTGGCCGTTAAACTATGGTTTC | GCTAACCGGCAATTTGATACCAAAG |
| 130619 | GTTTTTACCCGTCCTCGTGTCACTG | CAAAAATGGGCAGGAGCACAGTGAC |
| 144225 | ACAACAACACCGTCCTCGTGAGGTC | TGTTGTTGTGGCAGGAGCACTCCAG |
| 185975 | TCCGTCGCTGGGTGATGGAACATGA | AGGCAGCGACCCACTACCTTGTACT |
| 196417 | CCGGTCGTTGGTTGAGTGTTTATGA | GGCCAGCAACCAACTCACAAATACT |
| 223602 | ATTCCCAGGACACCACCAGAGGGCT | TAAGGGTCCTGTGGTGGTCTCCCGA |
| 229451 | ATCCGGTTAGGGAGTGTGTCCAATT | TAGGCCAATCCCTCACACAGGTTAA |
| 231682 | CGAGGACAGTAGGAACGGAGAACCC | GCTCCTGTCATCCTTGCCTCTTGGG |
| 242908 | TCTTGAAGAGGTCCCGCGACCGTGT | AGAACTTCTCCAGGGCGCTGGCACA |
| 249198 | TCAAGAACTAGGTCGCGACGAGGGT | AGTTCTTGATCCAGCGCTGCTCCCA |
| 252213 | GCGCGGCCCGGCTGGGCGTAGAGGG | CGCGCCGGGCCGACCCGCATCTCCC |
| 295450 | CCCGTCTTGCCTCGATAGGTAGGTC | GGGCAGAACGGAGCTATCCATCCAG |
| 306673 | GCCACTGCGGCTAGGACACTCGTAA | CGGTGACGCCGATCCTGTGAGCATT |
| 327300 | CTCTCAATGCGCTTTCCTCAGGAGG | GAGAGTTACGCGAAAGGAGTCCTCC |
| 468772 | ACGACGCAGGCAACTTCGTTGGCGG | TGCTGCGTCCGTTGAAGCAACCGCC |
| 529042 | GTCCGGACGGCAGATATCAGATACC | CAGGCCTGCCGTCTATAGTCTATGG |
| 561044 | GCACGTCACCAACCTTCGTAGTCGA | CGTGCAGTGGTTGGAAGCATCAGCT |
| 588830 | CGTATCTACAAACGGAGGAGAACCT | GCATAGATGTTTGCCTCCTCTTGGA |
| 604042 | AACTACTTCCACAGTGTTGCCGTTC | TTGATGAAGGTGTCACAACGGCAAG |
| 604967 | GTCCATCTCCACACTGGGGTCCTGA | CAGGTAGAGGTGTGACCCCAGGACT |
| 616095 | GTCCTACCACCACTAGTTGCACGGG | CAGGATGGTGGTGATCAACGTGCCC |
| 622161 | GACCCTCTGACACCCACTAGGAGGT | CTGGGAGACTGTGGGTGATCCTCCA |
| 622702 | GATAACCCACACAACATTTGTCTTC | CTATTGGGTGTGTTGTAAACAGAAG |
| 639146 | ACAAACAGACACGAAGATTTTCCAG | TGTTTGTCTGTGCTTCTAAAAGGTC |
| 669436 | CGGTCCGTCGGATATCCTTATCATT | GCCAGGCAGCCTATAGGAATAGTAA |
| 708420 | CGATCGGGTGAGGTGTCTAAAAACT | GCTAGCCCACTCCACAGATTTTTGA |
| 751418 | NA | NA |
| 752527 | ATTCCCACGATACGCGAGTCAGAGG | TAAGGGTGCTATGCGCTCAGTCTCC |
| 772367 | GCAAAGGGACACACTCCACCACGGT | CGTTTCCCTGTGTGAGGTGGTGCCA |
| 883312 | GTTCTCTGTACGTATTGTGCAAAAT | CAAGAGACATGCATAACACGTTTTA |
| 906039 | GTAAATAACTTGTTCGATCGGACAA | CATTTATTGAACAAGCTAGCCTGTT |
| 939909 | ACCTAGAGTCCTTTCGACGGTCGTA | TGGATCTCAGGAAAGCTGCCAGCAT |
| 1020364 | AGAGACGGGTTCCACTCCCTTGAGT | TCTCTGCCCAAGGTGAGGGAACTCA |
| 1059015 | CGACGTACAAGTATCAGTAGGTGGA | GCTGCATGTTCATAGTCATCCACCT |
| 1072725 | AAGAATTTCGTGGTGTAGTTGGAAT | TTCTTAAAGCACCACATCAACCTTA |
| 1099142 | ACATAGTGTCCTCTTTGGTCGTCCC | TGTATCACAGGAGAAACCAGCAGGG |
| 1331569 | CCCGGTGTCTAGTCCACGAAGACGT | GGGCCACAGATCAGGTGCTTCTGCA |
| 1354360 | GGAAGAGGTCTACTGCACCGACAGT | CCTTCTCCAGATGACGTGGCTGTCA |

TABLE 22

| Affymetrix | Transcript | SEQ | | | |
|---|---|---|---|---|---|
| Exon ID B | Cluster ID | Name | Strand | Strand Start | Strand Stop |
| 16650657 | 16650657 | — | — | — | — |
| 16650745 | 16650745 | — | — | — | — |
| 16651179 | 16651179 | — | — | — | — |
| 16651711 | 16651711 | — | — | — | — |
| 16651915 | 16651915 | — | — | — | — |
| 16651933 | 16651933 | — | — | — | — |
| 16652239 | 16652239 | — | — | — | — |
| 16652289 | 16652289 | — | — | — | — |
| 16652527 | 16652527 | — | — | — | — |
| 16652597 | 16652597 | — | — | — | — |
| 16653327 | 16653327 | — | — | — | — |
| 16655117 | 16655117 | — | — | — | — |
| 16655411 | 16655411 | — | — | — | — |
| 16656923 | 16656923 | — | — | — | — |
| 16657593 | 16657572 | chr1 | + | 911095 | 911203 |
| 16657854 | 16657848 | chr1 | + | 1543079 | 1543119 |
| 16658040 | 16658023 | chr1 | + | 2426362 | 2426386 |
| 16658687 | 16658674 | chr1 | + | 9671844 | 9671904 |
| 16659314 | 16659251 | chr1 | + | 12439570 | 12439594 |
| 16659467 | 16659459 | chr1 | + | 13922361 | 13922421 |
| 16659653 | 16659650 | chr1 | + | 15860772 | 15860796 |
| 16660293 | 16660282 | chr1 | + | 20411334 | 20411363 |
| 16660567 | 16660555 | chr1 | + | 22924244 | 22924268 |
| 16660942 | 16660933 | chr1 | + | 25889465 | 25889547 |
| 16661236 | 16661206 | chr1 | + | 26883177 | 26883201 |
| 16662358 | 16662357 | chr1 | + | 35661576 | 35661663 |
| 16662955 | 16662945 | chr1 | + | 40431586 | 40431686 |
| 16663887 | 16663871 | chr1 | + | 44684284 | 44684308 |
| 16666083 | 16666082 | chr1 | + | 71413125 | 71413151 |
| 16667412 | 16667385 | chr1 | + | 95712525 | 95712665 |
| 16667429 | 16667427 | chr1 | + | 95978079 | 95978160 |
| 16675846 | 16675844 | chr1 | + | 201804083 | 201804107 |
| 16676222 | 16676217 | chr1 | + | 203738776 | 203738809 |
| 16676511 | 16676498 | chr1 | + | 205569535 | 205569588 |
| 16676715 | 16676693 | chr1 | + | 207245602 | 207245626 |
| 16677217 | 16677201 | chr1 | + | 212274092 | 212274116 |
| 16679760 | 16679759 | chr1 | + | 248100578 | 248100658 |
| 16680781 | 16680770 | chr1 | − | 2979261 | 2979594 |
| 16684441 | 16684439 | chr1 | − | 31934171 | 31934242 |
| 16685867 | 16685866 | chr1 | − | 41154797 | 41157855 |
| 16685996 | 16685986 | chr1 | − | 42915653 | 42915677 |
| 16691349 | 16691333 | chr1 | − | 116311348 | 116311413 |
| 16696420 | 16696416 | chr1 | − | 173019898 | 173019989 |
| 16697659 | 16697654 | chr1 | − | 198906415 | 198906539 |
| 16698835 | 16698816 | chr1 | − | 208219352 | 208219376 |
| 16702313 | 16702311 | chr10 | + | 7830190 | 7830214 |
| 16708599 | 16708579 | chr10 | + | 104125274 | 104125298 |
| 16723812 | 16723803 | chr11 | + | 41857963 | 41857987 |
| 16730164 | 16730157 | chr11 | + | 93803602 | 93803707 |
| 16733477 | 16733473 | chr11 | + | 130058031 | 130058055 |
| 16740437 | 16740412 | chr11 | − | 65315216 | 65315240 |
| 16754738 | 16754729 | chr12 | + | 81537188 | 81537212 |
| 16756959 | 16756923 | chr12 | + | 110788494 | 110788518 |
| 16764284 | 16764238 | chr12 | − | 49504637 | 49504661 |
| 16783655 | 16783644 | chr14 | + | 39648245 | 39648280 |
| 16784915 | 16784909 | chr14 | + | 60733933 | 60733991 |
| 16787392 | 16787364 | chr14 | + | 90485693 | 90485717 |
| 16787649 | 16787642 | chr14 | + | 93359618 | 93359819 |
| 16792584 | 16792550 | chr14 | − | 50296038 | 50296062 |
| 16793638 | 16793632 | chr14 | − | 61447572 | 61447600 |
| 16801591 | 16801577 | chr15 | + | 59752184 | 59752276 |
| 16802685 | 16802681 | chr15 | + | 72102896 | 72102961 |
| 16804744 | 16804716 | chr15 | + | 90768778 | 90768802 |
| 16810304 | 16810298 | chr15 | − | 62936970 | 62937310 |
| 16819053 | 16819052 | chr16 | + | 55358040 | 55358655 |
| 16844154 | 16844137 | chr17 | − | 37557842 | 37557872 |
| 16851838 | 16851824 | chr18 | + | 29797774 | 29797878 |
| 16851883 | 16851866 | chr18 | + | 32409010 | 32409034 |
| 16890974 | 16890970 | chr2 | + | 219524492 | 219524516 |
| 16900045 | 16900044 | chr2 | − | 88529113 | 88529181 |
| 16900226 | 16900225 | chr2 | − | 95512287 | 95512384 |
| 16900670 | 16900657 | chr2 | − | 97613582 | 97613606 |
| 16907908 | 16907904 | chr2 | − | 213768128 | 213768160 |
| 16918801 | 16918797 | chr20 | − | 34214399 | 34214423 |
| 16922251 | 16922243 | chr21 | + | 34655474 | 34655536 |
| 16930003 | 16929994 | chr22 | + | 38363654 | 38363678 |
| 16939185 | 16939184 | chr3 | + | 38323792 | 38323817 |
| 16944985 | 16944982 | chr3 | + | 126006929 | 126006965 |
| 16945570 | 16945543 | chr3 | + | 130345367 | 130345391 |
| 16950391 | 16950390 | chr3 | − | 8406218 | 8406333 |
| 16956502 | 16956486 | chr3 | − | 84911918 | 84911942 |
| 16957913 | 16957906 | chr3 | − | 120360496 | 120360520 |
| 16964029 | 16964027 | chr4 | + | 1801084 | 1801233 |
| 17088090 | 17088084 | chr9 | + | 114409409 | 114409538 |

Several suitable statistical methods may be employed to compare the measured expression levels of genes from the plurality of genes listed in Tables 1-6 to control ranges disclosed herein. Non-limiting examples of suitable statistical methods that can be used for statistical analysis in the detection methods disclosed herein include the group consisting of Fisher Discriminant analysis (FDA), Diagonal linear discriminant analysis (DLDA), Linear discriminant analysis (LDA), linear discriminant analysis based on partial least-squares (PLSLDA), partial least squares random Forest (PLSRF), orthogonal discriminant analysis (QDA), Random Forest (RF), or a combination thereof. Examples of suitable statistical methods are listed in Table 1, for example Support Vector Machine, Quadratic Discriminant analysis, and Fisher's Discriminant analysis (FDA). It is noted that FDA has been shown to provide accurate results for both male and female subjects.

TABLE 7
Table: Overview of the classification models provided via CMA [2, 3-14].

| Method name | Package | Reference |
|---|---|---|
| Component wise Boosting | CMA | Bühlmann and Yu [2003] |
| Elastic Net | glmpath | Zhou and Hastie [2004] |
| Flexible Discriminant Analysis | mgcv | Ripley [1996] |
| Tree-based Boosting | gbm | Friedman [2001] |
| k-nearest neighbours | class | Ripley [1996] |
| Lasso | glmpath | Young-Park and Hastie |
| | | [2007] |
| Feed-Forward Neural Networks | nnet | Ripley [1996] |
| Probalistic nearest neighbours | CMA | — |
| Penalized Logistic Regression | CMA | Zhu [1996] |
| Probabilistic Neural Networks | CMA | Specht [1990] |
| PAM | CMA | Tibshirani et al [2002] |
| Shrinkage Discriminant Analysis | CMA | — |
| Support Vector Machine | e1071 | Schölkopf and Smola |
| | | [2002] |
| Diagonal Discriminant Analysis | CMA | McLachlan[1992] |
| Fisher's Discriminant Analysis | CMA | Ripley [1996] |
| Linear Discriminant Analysis | MASS | Mclachlan[1992] |
| Partial Least Squares | plsgenomics | Boulesteix and Strimmer |
| | | [2007] |
| ″ + Random Forest | plsgenomics | Boulesteix and Strimmer |
| | | [2007] |
| ″ + logistic regression | plsgenomics | Boulesteix and Strimmer |
| | | [2007] |
| Quadratic Discriminant Analysis | MASS | Mclachlan[1992] |
| Random Forest | randomForest | Breiman [2001] |

The Bioconductor library ‘CMA’ is used to make the prediction models and execute the validations [1, 2]. For the validation, a 10-fold cross validation is executed. In addition, the calculations (model generation and validation) are repeated 5 times to improve the validation measures.

### Detecting a Condition Comprising Perfusion Shortage or a Risk Thereof at an Early Stage

Conventional biomarkers in the field of cardiology or other areas where ischemia can cause serious conditions that are available to date have merely detected myocardial muscle damage after severe ischemia, but fail to detect subtle ischemia (a.k.a., angina pectoris) without muscle damage. Ischemia without muscle damage constitutes the vast majority of the cardiovascular patients in the normal cardiology outpatient clinic. Described in accordance with methods, kits, compositions, and uses of some embodiments herein are biomarkers of myocardial and cerebrovascular ischemia (or any other ischemic disease) in the absence of acute myo- or tissue necrosis (including, but not limited to, angina pectoris, transient ischemic (cerebrovascular) accident, microvascular disease and/or cerebrovascular accident). Further, in certain aspects, the present disclosure presents methods comprising obtaining said biological sample from a subject that has or is at risk for having a condition comprising perfusion shortage, wherein said perfusion shortage is at an early stage and has not caused any detectable tissue damage and assaying expression levels of two or more genes (nucleotide or protein biomarker) from a plurality of identified genes (nucleotide or protein biomarker) to generate an output comprising said expression levels indicative of said subject's having or risk of having said condition comprising perfusion shortage.

It is contemplated that methods, compositions, kits, and uses of some embodiments can detect conditions comprising perfusion shortage such as neoangiogenesis or ischemia, even when the subject presents minimal to no symptoms of the condition. In some embodiments, the subject may not present symptoms of any of ischemic cardiovascular disease, microangiopathy in cardiovascular disease (such as diastolic heart failure), hypertrophic cardiomyopathy, cardiovascular pathology with relative ischemia (such as hypertrophic CMP, ischemic heart failure, and/or congenital cardiac disease), ischemic cerebrovascular disease (including CVA/TIA, and/or vascular dementia), ischemic peripheral artery disease, venous disease, orthopedic disease, sepsis, inflammation, lymphatic disease, ophthalmological disease associated with ischemia and/or neoangiogenesis (such as neoangiogenesis in retinopathy), diabetic macro and microangiopathy, oncological pathology (including solid tumors, metastatic disease), endothelial dysfunction in cardiovascular and peripheral artery disease, ophthalmology (such as neoangiogenesis in retinopathy), diabetic macro and microangiopathy, oncological pathology (such as solid tumors and/or metastatic disease), endothelial dysfunction in cardiovascular and peripheral artery disease, nephrological disease associated with ischemia and/or neoangiogenesis, gynecological disease, orthopedic disease, lung disease (such as COPD, emphysema, lung fibrosis, cystic fibrosis, lung cancer, pulmonary interstitial disease, and/or monitoring pre- and post-lung transplantation, pulmonary emboli), cerebrovascular disease (for example, stroke, TIA, microvascular dementia, and emboli such as pulmonary emboli), deep venous thrombosis, inadequate ventilation/oxygenation of a mechanically ventilated subject, a gastrointestinal disorder (such as Crohn's disease, colitis ulcerosa), a neurodegenerative disease (such as Parkinson's disease or multiple sclerosis), erectile dysfunction, sport injury, frailty, shock (such as post-resuscitation shock), arterial and venous diseases, disease related to lymphangiogenic disease, vascular remodeling including lymphangiogenic metastatic disease, arteriovenous malformation, arterial remodeling, or venous thrombosis, or a combination of two or more of the listed items. In some embodiments, the subject may present symptoms of any of ischemic diseases disclosed herein or any other ischemic diseases.

### Measuring or Detecting Expression Levels of Selected Genes

As used herein “gene products” refer to DNA molecules, RNA molecules (such as mRNAs), particular splice variants, corresponding (translated) peptides, and/or specific downstream signaling cascades (for example, phosphorylation, acetylation, methylation, ubiquitination, SUMOylation, degradation, or subcellular localization of downstream signal transduction pathway molecules), or any combination of two or more of the listed items (any of these items may be referred to as biomarkers). Genes as disclosed herein can be measured or detected by measuring a portion of DNA molecules, RNA molecules (such as mRNAs), particular splice variants, or corresponding (translated) peptides corresponding to a particular gene from the identified set of genes. Gene products/genes that are differently expressed in ischemia and non-ischemia subjects as described herein may also be referred to as “biomarkers.” For nucleic acid gene products/genes (or nucleic acids encoding gene products/genes), it will be appreciated that the reverse transcript of a gene product/gene also provides suitable information about the gene product. Accordingly, gene product/gene can refer to either strand of a nucleic acid (e.g., the sense or antisense strand), and thus encompasses the reverse complement of a nucleic acid strand as well as the strand itself, in any reading frame on either strand.

In methods, compositions, kits, and uses of some embodiments, the specified gene products can identify diseases comprising perfusion shortage (such as ischemic episodes and neoangiogenesis processes) based on expression levels of these gene products/genes, such as DNA molecules, RNA molecules (e.g., mRNAs), particular splice variants, corresponding (translated) peptides, and/or specific downstream signaling cascades (which can be measured, for example, based on phosphorylation, ubiquitination, SUMOylation, transcription, degradation, or subcellular localization of downstream signal transduction pathway molecules), or any combination of two or more of the listed items. In some embodiments, the gene products comprise nucleic acids that are part of the genes selected from Tables 1-6. In some embodiments, the gene products comprise nucleic acids that may be used to detect the expression levels of genes selected from Tables 1-6. In some embodiments, the gene products comprise peptide sequences that are encode from the genes selected from Tables 1-6. In some embodiments, the gene products comprise peptides that can detect expression levels of protein or fragments thereof encoded by the genes selected from Tables 1-6. In some embodiments, the gene products comprise nucleic acids or peptide sequences encoded therefrom selected from any of SEQ ID NOs: 1-5103 (or a peptide sequence encoded therefrom), Genes Number 1-9280 (or a peptide sequence encoded therefrom), or nucleic acids comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 consecutive nucleotides thereof (including ranges between any two of the listed values), or peptides encoded by any of these nucleic acids. It has been observed that RNAs or peptides of each of Tables 1, 3, and 5 are differentially expressed in hematopoietic EPCs or fractions or secretions thereof of females, and that RNAs or peptides of each of Tables 2, 4, and 6 are differentially expressed in hematopoietic EPCs or fractions or secretions thereof of males. As such, in some embodiments, for example for female subjects the gene products comprise nucleic acids or peptides selected from any of Tables 1, 3, and 5, or nucleic acids comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 consecutive nucleotides thereof (including ranges between any two of the listed values), or peptides encoded by any of these nucleic acids. In some embodiments, for example for male subjects the gene products comprise nucleic acids or peptides selected from any Tables 2, 4, and 6, or nucleic acids comprising at least 2, 3, 4, 5, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 consecutive nucleotides thereof (including ranges between any two of the listed values), or peptides encoded by any of these nucleic acids.

Transcriptomics refers to global analysis of RNA expression and may elucidate genes and molecular pathways involved in biological processes. Genes showing similarities in expression pattern may be functionally related and fall under the same genetic control mechanism. Furthermore, it will be appreciated that transcriptomics can identify particular splice variants, and that splice variants can also be useful as biomarkers, as transcripts may be spliced differently under different conditions. Furthermore, it will be appreciated that transcriptomics can identify gene products such as peptides that are also useful as biomarkers, since relative levels of transcripts can correlate with, and predict, relative levels of peptides. Using transcriptomics technology, specific expression patterns of gene products have been identified, which were up-regulated under hypoxic conditions in patients with conditions comprising perfusion shortage, such as (but not limited to) myocardial ischemia (due to oxygen deprivation in the heart, which may also be referred to as angina pectoris), peripheral artery ischemia, cerebrovascular ischemia (stroke and TIA), & retinal ischemia (See Example 2 and Tables 1-6). Without being limited by theory, this altered regulation is suggestive for their involvement in a physiological ischemia-driven neoangiogenesis response.

Levels of gene products/genes (which may also be referred to herein as “levels of expression of genes/gene products” or “expression levels of genes/gene products” or “levels of biomarkers” or “levels of expression of biomarkers” or “expression levels of biomarkers”, including variations of these root terms) may be detected in accordance with methods, compositions, kits, and uses using suitable art-recognized methods. Methods, compositions, kits, and uses of some embodiments utilize gene expression profiling to identify biomarkers that exhibit differential gene expression (of DNA sequences/RNA oligonucleotide sequences) and hence DNA/RNA molecular biomarkers, as well as corresponding splice variants, peptides, and/or downstream signal transduction markers. It is noted that for a given nucleic acid sequence, a peptide may be translated from the +1, +2, or +3 position on either the sense or the antisense strand. Accordingly, a peptide corresponding to a given nucleic acid will be understood to encompass translational initiation at position +1, +2, or +3 on either of the sense or antisense stands.

It is contemplated that any suitable detection platform for DNA, RNA, peptide, and/or signal transduction biomarkers can be used in accordance with methods, compositions, uses, and kits of embodiments herein. The detection platform may have sufficient sensitivity to discriminate between levels of gene products, such as nucleic acids or peptides of any of SEQ ID NOs: 1-5103 or Genes Number 1-9280, as described herein, or corresponding peptides. For example, the detection of the peptide biomarkers or small molecules may be performed by for example, (but not limited to) ELISA, (quantitative) western blot analysis, mass spectrometry, flow cytometry, immunohistochemistry, and/or protein array, or a combination of two or more of any of the listed items. In some embodiments, ChipArray technology is used to detect expression levels of genes/genes products as described herein. In some embodiments, other technologies can be used to determine differential gene expression with sufficient sensitivity to form the basis for this assay. In some embodiments, a molecular biomarker profile can be detected irrespective of the underlying (current or future) platform, provided the technology has sufficient sensitivity to pick up this low-level differential expression. These may include (but are not limited to) qRT PCR analysis, (quantitative) Northern Blot analysis, microarray analysis, Luminex technology, Multiplexing, SAGE, RNASeq, whole transcriptome analysis, or a combination of any of these listed items. The detected expression levels of gene products in accordance with methods, compositions, kits, and uses of embodiments herein may be compared to a control range. As used herein a “control range” has its ordinary and customary meaning as would be understood by one of ordinary skill in the art in view of this disclosure. It refers to a range of expression levels of a gene/gene product in a sample of a subject that does not have the condition comprising neoangiogenesis or ischemia, for example a healthy subject. It will be appreciated that a control range can be determined experimentally (for example by detecting gene expression levels in a sample of a healthy subject or by using standard samples having gene expression levels indicative of an absence of the condition indicates a level of expression of the gene product outside of the control range) or can be obtained from an electronically or optically stored set of values (for example, from a database of control ranges). By way of example, a control range may be provided as a numerical range, or a confidence interval around a specified value. In some embodiments, an expression level of a gene product exceeds the upper limit of a control range and indicates a presence or risk of the condition comprising neoangiogenesis and/or ischemia. By way of example, expression levels of genes/gene products that are indicative of a presence or risk of the condition comprising neoangiogenesis and/or ischemia are shown in Tables 1-6. In some embodiments, if an expression level of a gene product is greater than or equal to a value in an ischemia patient of Tables 1, 3, and 5 and/or Table 2, 4, and 6, a presence or risk of the condition comprising neoangiogenesis and/or ischemia is determined. In some embodiments, the gene product does not comprise the product of any of Hspg2, Cyb5b, Adora2a, LDLRAD2, or Lgmn. In some embodiments, the gene product does not comprise the product of any of Sox18, Sox7, H01, ETS2, Rab5, Rock2, Rin3, Crip2, Kcnh2, Plvap, TNFAIP8L1, STAB1, C10orf10, Cingulin like 1, ExoC3I, CCBE1, KIAA1462, 1200015N20Rik, Agtrl-1, DLL4, or HEY1.

In some embodiments, one or more of a copy number of the at least two specified different gene products, an expression level of the at least two specified different gene products are detected, or an activity level of the at least two specified different gene products is detected.

In the method of some embodiments, the specified different gene products comprise DNA or RNA comprising one or more exons that individually or in combination comprise a sequence of Table 3 or Table 4.

The methods, compositions, kits, and uses of some embodiments (which may comprise an array as described herein) can be used for side-by-side comparison between different (anti-ischemic) therapies to demonstrate superiority or non-inferiority (in for instance clinical trials as a surrogate end-point marker). Likewise, methods, compositions, uses, kits of some embodiments may be useful, and exhibit alike properties in diseases where (the lack of or excess) tissue perfusion plays a substantial pathogenetic role or determines therapy responsiveness, including (but not limited to) oncological disease (for instance hematological metastatic disease, solid tumors), diabetic vascular complications (macro and microangiopathy), diastolic heart failure, ophthalmic neoangiogenesis disease, and renal disease, including combinations of two or more of any of the listed items. The methods, compositions, kits, and uses may comprise an array as described herein.

In some embodiments (for example, methods, compositions, kits, and uses as described herein), the expression levels of genes/gene products are assessed as levels of DNA, RNA, peptide, or any combinations thereof. The expression levels of DNA or RNA can be assessed for example by (but not limited to) cDNA chip array analysis, quantitative PCR (qPCR, such as quantitative RT PCR), microarray analysis, multiplexing, Luminex analysis, nucleic acid sequencing, northern blot analysis, comparative genomic hybridization (CGH), and/or fluorescent in situ hybridization (FISH), genomic high throughput sequencing, nanostring, massive parallel signature sequencing (MPSS), serial analysis of gene expression (SAGE), or a combination of two or more of the listed items. Further, the expression levels of peptides can be assessed for example by (but not limited to) enzyme-linked immunosorbent assay (ELISA), protein sequencing, mass spectrometry (such as MALDI TOF, QTOF, or SELDI TOF), quantitative western blot analysis, and peptide barcoding, or a combination of two or more of the listed items. In methods, compositions, kits, and uses of some embodiments, the expression levels of genes/gene products are assessed for example by (but not limited to) cDNA chip array analysis, quantitative PCR (qPCR, such as quantitative RT PCR), microarray analysis, multiplexing, Luminex analysis, nucleic acid sequencing, northern blot analysis, comparative genomic hybridization (CGH), and/or fluorescent in situ hybridization (FISH), genomic high throughput sequencing, nanostring, massive parallel signature sequencing (MPSS), serial analysis of gene expression (SAGE), enzyme-linked immunosorbent assay (ELISA), protein sequencing, mass spectrometry (such as MALDI TOF, QTOF, or SELDI TOF), quantitative western blot analysis, and peptide barcoding, or a combination of two or more of the listed items. In some embodiments, the expression levels of the one or more gene products are assessed by detecting the presence in the samples of a polynucleotide molecule encoding the target or a portion of the polynucleotide molecule. In some embodiments, the polynucleotide molecule is a mRNA, cDNA, or functional variants or fragments thereof. Optionally, the detecting can further comprise amplifying the polynucleotide molecule. In some embodiments, the expression level of the one or more gene products is assessed by annealing a nucleic acid probe with the sample of the polynucleotide encoding the one or more targets or a portion of the polynucleotide molecule under stringent hybridization conditions. In some embodiments, the expression level of the gene products is assessed by detecting the presence in the samples of a protein of the target, a peptide, or protein fragment thereof comprising the protein. In some embodiments, the presence of the protein, peptide or protein fragment gene products is detected via mass spectrometry or using a reagent which specifically binds to the protein, peptide or protein fragment thereof. Examples of suitable reagents include, but are not limited to an antibody (as used herein, “antibody” encompasses full-length antibodies, as well as derivatives and binding fragments thereof, for example Fabs, scFvs, minibodies, diabodies, triabodies, and the like) and/or an aptamer (such as a nucleic acid or peptide aptamer). It will be appreciated that gene activity can be modulated by directly or indirectly targeting a gene product. For example, gene activity can be enhanced or increased by increasing the copy number of a gene product (or a nucleic acid encoding the gene product), by increasing the stability of a gene product (or a nucleic acid encoding the gene product), and/or by changing the location of a gene product (for example by translocating a gene product to a location where it acts, or translocating an mRNA or a ribosome). For example, gene activity can be inhibited of decreased by decreasing the copy number of a gene product (or a nucleic acid encoding the gene product), by decreasing the stability of a gene product (or a nucleic acid encoding the gene product), and/or by changing the location of a gene product (for example by sequestering a gene product or a nucleic acid encoding a gene product, or targeting a peptide gene product to a proteasome). In some embodiments, the expression level of the gene product is assessed by determining the magnitude of modulation of the activity or expression level of downstream targets of the gene product. In some embodiments, for example, an at least a 5%, 10%, 15%, or 20%, increase or an at least 5%, 10%, 15%, or 20%, decrease between the copy number, level of expression, or level of activity of the one or more downstream targets in the subject sample relative to the copy number, level of expression, or level of activity of the one or more downstream targets in a control sample indicates a level of expression of the gene product outside of the control range. In some embodiments, for example, a less-than 20%, 15%, 10%, or 5% increase or a less-than 20%, 15%, 10%, or 5% decrease between the copy number, level of expression, or level of activity of the one or more downstream targets in the subject sample relative to the copy number, level of expression, or level of activity of the one or more downstream targets in a control sample indicates a level of expression of the gene product within the control range. In methods, compositions, kits, and uses of some embodiments, expression levels of the specified different gene products are detected. For example, detection can be for example (but not limited to) any of cDNA chip array analysis, quantitative PCR (qPCR) such as quantitative RT PCR, microarray analysis, comparative genomic hybridization (CGH), fluorescent in situ hybridization (FISH), multiplexing, Luminex analysis, nucleic acid sequencing, northern blot analysis, (genomic) high throughput sequencing, nanostring, massive parallel signature sequencing (MPSS), serial analysis of gene expression (SAGE), enzyme-linked immunosorbent assay (ELISA), protein sequencing, mass spectrometry (such as MALDI TOF, QTOF, or SELDI TOF), quantitative western blot analysis, and peptide barcoding, Affymetrix arrays, GeneChip arrays, tiling arrays, anti-sense arrays, spotted DNA arrays, SNP arrays, expression arrays, or a combination thereof. In methods, compositions, kits, and uses of some embodiments, the expression levels of gene products are assessed for example (but not limited), by microarray analysis, qPCR, high-throughput sequencing, CGH, and/or FISH.

In some embodiments, an agent for detecting target mRNA, genomic DNA, or fragments thereof is a labeled nucleic acid probe capable of hybridizing to target mRNA, genomic DNA, or fragments thereof. The nucleic acid probe can be, for example, full-length target nucleic acid, or a portion thereof, such as an oligonucleotide of at least 5, 6, 7, 8, 9, 10, 15, 16, 17, 18, 19, 20, 21, 22, 23, 24, 25, 26, 27, 28, 29 30, 31, 32, 33, 34, 35, 40, 45, 50, 60, 70, 80, 90, 100, 150, 200, 250, or 500, or a range defined by any two of the preceding values (such as 10-40, 10-30, 10-25, 15-40, 15-30, 15-25, 20-40, 20-30, or 20-25) nucleotides in length and sufficient to specifically hybridize under stringent conditions well known to a skilled artisan to target mRNA or genomic DNA. Other suitable probes for use in the diagnostic assays of the disclosure are described herein. It will be appreciated that a “probe” can comprise or consist essentially of or consist of a primer of a primer pair. In some embodiments, the nucleic acid probe is designed to detect transcript variants (e.g., different splice forms) of a gene. Optionally, any nucleic acid probe as described herein can further comprise a detectable moiety, for example a fluorophore, enzyme, radiolabel, quantum dot, or a nucleic acid barcode. Examples of fluorophores include, but are not limited to xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, CalFluorOrange.

As used herein “probe” refers to a nucleic acid that hybridizes to a target nucleic acid sequence. For example, the probe may be immobilized on a substrate and may hybridize with (and capture) a target nucleic acid. For example, the probe may be part of a primer pair as described herein, or may be used for quantification in conjunction with nucleic acid amplification, for example in qPCR. For example, the probe may comprise a detectable moiety such as a fluorophore, or a fluorophore-quencher pair, such as in a TaqMan probe. In some embodiments, detection of the levels of expression of gene products (such as peptides, or RNA's such as miRNAs, non-coding RNAs, coding RNAs, and mRNAs) involves the use of a probe/primer in a polymerase chain reaction (PCR) (see, e.g., U.S. Pat. Nos. 4,683,195 and 4,683,202), such as anchor PCR or RACE PCR, or, in some embodiments, in a ligation chain reaction (LCR) (see, e.g., Landrigan et al. (1988) Science 241: 1077-1080; and Nakazawa et al. (1994) Proc. Natl. Acad. Sci. USA 91:360-364), the latter of which can be particularly useful for detecting point mutations in one or more targets gene (see Abravaya et al. (1995) Nucleic Acids Res. 23:675-682). This method can include the steps of collecting a sample of cells from a subject, isolating nucleic acid (e.g., genomic DNA, mRNA, cDNA, small RNA, mature miRNA, pre-miRNA, pri-miRNA, miRNA*, anti-miRNA, or a miRNA binding site, or a variant thereof) from the cells of the sample, contacting the nucleic acid sample with one or more primers which specifically hybridize to one or more targets gene of the disclosure, including the target genes selected from Tables 1-6, or fragments thereof, under conditions such that hybridization and amplification of the target gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting levels of gene products as described herein.

In some embodiments, expression levels of the specified different genes/gene products are detected. In some embodiments, detecting expression levels of the specified different genes/gene products comprises a technique selected from the group, but not limited to, consisting of cDNA chip array analysis, quantitative (RT) PCR, microarray analysis, multiplexing, Luminex analysis, nucleic acid sequencing, northern blot analysis, genomic high throughput sequencing, nanostring, massive parallel signature sequencing (MPSS), and serial analysis of gene expression (SAGE), or a combination of two or more of the listed items. Optionally, the detecting method comprises a technique selected from the group consisting of, but not limited to, enzyme-linked immunosorbent assay (ELISA), protein sequencing, mass spectrometry (such as MALDI TOF, QTOF, or SELDI TOF), quantitative western blot analysis, and peptide barcoding, or a combination of two or more of the listed items.

In some embodiments, detecting expression levels of the specified different genes/gene products comprises detecting transcript levels, detecting peptide levels, or detecting signal transduction activity for each of the specified different gene products.

In some embodiments, detecting expression levels of the specified different genes/gene products comprises contacting the sample with a probe that hybridizes to at least a portion of a transcript of a gene, the transcript comprising any of nucleotide sequences listed in Tables 3 and 4, or an antibody that binds specifically to a peptide listed in Tables 3 and 4. At least a portion of the peptide can be encoded by a nucleotide sequence selected from Tables 3 and 4, and the method can comprise for example (but not limited to), protein sequencing, mass spectrometry (such as MALDI TOF, QTOF, or SELDI TOF), or peptide barcoding.

In some embodiments, detecting expression levels of the specified different genes/gene products comprises contacting the sample with a probe that hybridizes to at least a portion of a transcript of a gene, the transcript comprising any of nucleotide sequences listed in Tables 3 and 4.

In some embodiments, detecting expression levels of the specified different genes/gene products comprises contacting the sample with an antibody that binds specifically to a peptide. At least a portion of the peptide can be encoded by a sequence selected from peptide sequences listed in Tables 3 and 4.

In some embodiments, detecting expression levels of the specified different genes/gene products comprises peptide analysis, for example (but not limited to), protein sequencing, mass spectrometry (such as MALDI TOF, QTOF, or SELDI TOF), or peptide barcoding.

In some embodiments, detecting expression levels of the specified different genes/gene products comprises one or more of contacting the sample with a probe that hybridizes to at least a portion of a transcript of a gene (the transcript comprising any of nucleotide sequences listed in Tables 3 and 4) and/or an antibody that binds specifically to a peptide (in which at least a portion of the peptide is encoded by a sequence selected from Tables 3 and 4); and/or, for example (but not limited to) protein sequencing, mass spectrometry (such as MALDI TOF, QTOF, or SELDI TOF), or peptide barcoding.

In some embodiments, detecting expression levels of the specified different genes/gene products comprises contacting the sample with at least two probes that each hybridize to at least a portion of a transcript of a gene, the transcript comprising any of nucleotide sequences listed in Tables 3 and 4, or at least two antibodies (or protein ligands) that each bind specifically to a peptide, in which at least a portion of the peptide is encoded by a sequence selected from Tables 3 and 4.

In some embodiments, detecting expression levels of the specified different genes/gene products comprises contacting the sample with at least two probes that each hybridize to at least a portion of a transcript of a gene or the reverse complement thereof, the transcript comprising any of nucleotide sequences listed in Tables 3 and 4.

In some embodiments, detecting expression levels of the specified different genes/gene products comprises contacting the sample with at least two antibodies (or protein ligands) that each bind specifically to a peptide, in which at least a portion of the peptide is encoded by a sequence selected from Tables 3 and 4.

In some embodiments, detecting expression levels of the specified different genes/gene products comprises contacting the sample with at least 6, 7, 8, 9, 10, 11, 12, 13, 14, 15, 16, 17, 18, 19, 25, 30, or 35 but no more than 40 (or 20 or 15 or 14) probes that each hybridize to at least a portion of a gene product (such as a transcript of a gene) comprising any of nucleotide sequences listed in Tables 3 and 4; and/or at least 2, 3, 4, 5, 6, 7, 8, 9, 10, 11, 12, 13, 14, 15, 16, 17, 18, 19, 20, 25, 30, or but no more than 40 (or 20 or 15 or 14) antibodies (or protein ligands) that each bind specifically to a peptide, wherein at least a portion of the peptide is encoded by a sequence selected from Tables 3 and 4.

In some embodiments, detecting expression levels of the specified different genes/gene products comprises contacting the sample with at least 6, 7, 8, 9, 10, 11, 12, 13, 14, 15, 16, 17, 18, 19, 25, 30, or 35 but no more than 40 (or 20 or 15 or 14) probes that each hybridize to at least a portion of gene product (such as a transcript of a gene), comprising any of nucleotide sequences listed in Tables 3 and 4.

In some embodiments, detecting expression levels of the specified different genes/gene products comprises contacting the sample with at least 6, 7, 8, 9, 10, 11, 12, 13, 14, 15, 16, 17, 18, 19, 25, 30, or 35 but no more than 40 (or 20 or 15 or 14) antibodies (or protein ligands) that each bind specifically to a peptide, wherein at least a portion of the peptide is encoded by a sequence selected from Tables 3 and 4.

In some embodiments, detecting expression levels of the specified different genes/gene products comprises contacting the sample with (a) for a female subject, a set of at least 6, but no more than 60 (for example, 6-14, 6-15, 6-20, 6-25, 6-30, 6-35, 6-40, 6-50, 6-60, 8-14, 8-15, 8-20, 8-25, 8-30, 8-35, 8-40, 8-50, 8-60, 9-10, 9-14, 9-15, 9-16, 9-20, 9-40, 9-50, 9-60, 10-14, 10-15, 10-20, 10-25, 10-10-35, 10-40, 10-50, or 10-60) different nucleic acid probes that each hybridize to at least a portion of a gene product (such as a transcript of a gene) comprising any of nucleotide sequences listed in Table 3, (b) for a male subject, a set of at least 6, but no more than 40 (for example, 6-14, 6-15, 6-20, 6-25, 6-30, 6-35, 6-40, 8-14, 8-15, 8-20, 8-25, 8-30, 8-35, 8-40, 9-10, 9-14, 9-15, 9-16, 9-20, 9-40, 10-14, 10-20, 10-25, 10-30, 10-35, or 10-40) different nucleic acid probes and/or primer pairs that each hybridize to at least a portion of a transcript of a gene or the reverse complement thereof, the transcript comprising any of nucleotide sequences listed in Table 4, (c) for a female subject, a set of at least 6, but no more than 12 (for example, 6-12, 6-10, 6-8, 8-12, 8-10, or 10-12) different antibodies (or protein ligands) that each bind specifically to a peptide encoded by sequences selected from Table 3, and/or (d) for a male subject, a set of at least 14, but no more than 20 (or a set of no more than 15) different antibodies (or protein ligands) that each bind specifically to a peptide encoded by sequences selected from Table 4. In some embodiments, the same numerical range of gene products is detected for male and female subjects.

In some embodiments, for a female subject, detecting expression levels of the specified different genes/gene products comprises contacting the sample with a set of at least 6, but no more than 30, or 20 different nucleic acid probes and/or primer pairs that each hybridize to at least a portion of a transcript of a gene or the reverse complement thereof, the transcript comprising any of nucleotide sequences listed in Tables 3.

In some embodiments, fora male subject, detecting expression levels of the specified different genes/gene products comprises contacting the sample with a set of at least 10, but no more than 40, 30, or 20 different nucleic acid probes and/or primer pairs that each hybridize to at least a portion of a transcript of a gene or the reverse complement thereof, the transcript comprising any of nucleotide sequences listed in Table 4.

In some embodiments, for a female subject, detecting expression levels of the specified different genes/gene products comprises contacting the sample with a set of at least 10, but no more than 40, 30, or 20 different antibodies (or protein ligands) that each bind specifically to a peptide encoded by sequences selected from Table 3.

In some embodiments, fora male subject, detecting expression levels of the specified different genes/gene products comprises contacting the sample with set of at least 6, 7, 8, 9, 10, 11, 12, 13, 14, 16, 17, 18, 19, 20, 25, 30, or 35 but no more than 40 different antibodies that each bind specifically to a peptide encoded by sequences selected from Table 4. In some embodiments, for a male subject, detecting expression levels of the specified different genes/gene products comprises contacting the sample with set of at least 6, 7, 8, 9, 10, 11, 12, 13, 14, 15, 16, 17, 18, 19, 20, 25, 30, or 35 but no more than 40 different antibodies (or protein ligands) that each bind specifically to a peptide encoded by sequences selected from Table 4.

In some embodiments, for a female subject, detecting expression levels of the specified different genes/gene products comprises contacting the sample with one or more of a set of at least 6, 7, 8, 9, 10, 11, 12, 13, 14, 15, 16, 17, 18, 19, 20, 25, 30, or 35 but no more than 40 different nucleic acid probes (for example, a part of primer pairs, or for capture on an array) that each hybridize to at least a portion of a transcript of a gene or the reverse complement thereof, the transcript comprising any of nucleotide sequences listed in Table 3, and/or a set of at least 6, 7, 8, 9, 10, 11, 12, 13, 14, 15, 16, 17, 18, 19, 20, 25, 30, or 35 but no more than 40 different antibodies (or protein ligands) that each bind specifically to a peptide encoded by sequences selected from Table 3.

In some embodiments, fora male subject, detecting expression levels of the specified different genes/gene products comprises contacting the sample with one or more of a set of at least 10, 11, 12, 13, 14, 15, 16, 17, 18, 19, or 20 but no more than 40 different nucleic acid probes and/or primer pairs that each hybridize to at least a portion of a transcript of a gene or the reverse complement thereof, the transcript comprising any of nucleotide sequences listed in Table 4, and/or a set of at least 10, 11, 12, 13, 14, 15, 16, 17, 18, 19, or 20, but no more than 40 different antibodies (or protein ligands) that each bind specifically to a peptide encoded by sequences selected from Table 4. In some embodiments, for a male subject, detecting expression levels of the specified different genes/gene products comprises contacting the sample with one or more of a set of at least 8, 9, 10, 11, 12, 13, or 14, but no more than different nucleic acid probes and/or primer pairs that each hybridize to at least a portion of a transcript of a gene or the reverse complement thereof, the transcript comprising any of sequences selected from Table 4, and/or a set of at least 8, 9, 10, 11, 12, 13, or 14, but no more than 15 different antibodies (or protein ligands) that each bind specifically to a peptide encoded by sequences selected from Table 4.

In some embodiments, for a female subject, detecting expression levels of the specified different genes/gene products comprises contacting the sample with a set of at least 6, 7, 8, 9, 10, or 11, but no more than 12 (for example, 6-12, 6-10, 6-8, 8-12, 8-10, or 10-12) different nucleic acid probes and/or primer pairs that each hybridize to at least a portion of a transcript of a gene or the reverse complement thereof, the transcript comprising any of nucleotide sequences listed in Table 3. Optionally, detecting expression levels of the specified different genes/gene products comprises contacting the sample with a set of at least 6, 7, 8, 9, 10, 11, or 12, but no more than 12 (for example, 6-12, 6-10, 6-8, 8-12, 8-10, or 10-12) different antibodies (or protein ligands) that each bind specifically to a peptide encoded by sequences selected from Table 3.

In some embodiments, fora male subject, detecting expression levels of the specified different genes/gene products comprises contacting the sample with a set of at least 10, 11, 12, 13, 14, 15, 16, 17, 18, or 19, but no more than 20 (or no more than 15) different nucleic acid probes and/or primer pairs that each hybridize to at least a portion of a transcript of a gene or the reverse complement thereof from Tables 2, 4, and 6. Optionally, detecting expression levels of the specified different genes/gene products comprises contacting the sample with a set of at least 10, 11, 12, 13, 14, 15, 16, 17, 18, or 19, but no more than 20 (or no more than 15) different antibodies (or protein ligands) that each bind specifically to a peptide encoded by sequences selected from Tables 2, 4, and 6.

Subjects Having or at Risk of Having Conditions Comprising Neoangiogenesis and/or Ischemia

As used herein, a subject is referred to a mammal, including, for example, a murine, porcine, bovine, equine, canine, and/or feline mammals. In some embodiments, a subject is a non-human primate. In some embodiments, the subject is a human. In some embodiments, the subject is a healthy subject. In some embodiments, the subject is a mammal with a cardiac disease or a cardiovascular condition. In some embodiments the subject is a mammal at risk of having a cardiac disease or a cardiovascular condition. In some embodiments, the subject is at risk of ischemia. In some embodiments, the subject is a mammal with a cardiovascular condition characterized by perfusion shortage, or at a risk thereof. In some embodiments, the subject has a cardiac disease. In some embodiments the subject has a disease, or a cardiovascular condition characterized by oxygen insufficiency related to a cardiovascular event.

It is contemplated that methods, compositions, kits, and uses of embodiments herein can be used to detect any art-recognized condition comprising neoangiogenesis or ischemia or the risk thereof. Additionally, methods, compositions, kits, and uses comprising a therapeutic compound and/or medical intervention as described herein can be used to inhibit, ameliorate, delay the onset of, reduce the likelihood of, treat, or prevent any art-recognized condition comprising neoangiogenesis or ischemia.

In certain embodiments, a subject may be diagnosed with one of the diseases or conditions listed here. Alternatively, a subject may be given an output/report that the subject is at risk of having one of the diseases or conditions listed herein. “Disease” and “condition” comprising perfusion shortage are used interchangeably herein. Examples of conditions (or diseases) comprising perfusion shortage include ischemic diseases (such as for example (but not limited to) cardiovascular ischemic disease such as angina pectoris, silent myocardial ischemia, microvascular cardiovascular disease, myocardial infarction, TIA, stroke, minimal vessel dementia, peripheral artery disease) and disease with a perfusion component (for example (but not limited to), cancer, diabetes, and ophthalmologic diseases). By way of example, cancer can comprise perfusion components such as tumor vascularization and/or hematological or lymphogenic metastasis (and thus can be characterized as a disease comprising a perfusion shortage or deficiency and neoangiogenesis (tumor angiogenesis)). By way of example, diabetes, in which there can be challenges in providing blood to the extremities can be characterized as a perfusion shortage or retinal neoangiogenesis. By way of example, ophthalmologic conditions for example (but not limited to) retinal neoangiogenesis can be characterized as a neoangiogenesis. By way of example, gynecological conditions such as placental buildup during a menstrual cycle can be characterized as a perfusion shortage. Accordingly, examples of conditions or diseases comprising perfusion shortage include, but are not limited to, ischemic cardiovascular disease, microangiopathy in cardiovascular disease (such as diastolic heart failure), hypertrophic cardiomyopathy, cardiovascular pathology with relative ischemia (such as hypertrophic CMP, ischemic heart failure, and/or congenital cardiac disease), myocardial infarction, ischemic cerebrovascular disease (including CVA/TIA, and/or vascular dementia), ischemic peripheral artery disease, venous disease, orthopedic disease, sepsis, inflammation, lymphatic disease, ophthalmological disease associated with ischemia and/or neoangiogenesis (such as neoangiogenesis in retinopathy), diabetic macro- and microangiopathy, oncological pathology (including solid tumors (tumor angiogenesis), metastatic disease), endothelial dysfunction in cardiovascular and peripheral artery disease, ophthalmology (such as neoangiogenesis in retinopathy), diabetic macro- and microangiopathy, oncological pathology (such as solid tumors and/or metastatic disease), endothelial dysfunction in cardiovascular and peripheral artery disease, nephrological disease associated with ischemia and/or neoangiogenesis, gynecological disease, orthopedic disease, lung disease (for example (but not limited to) COPD, emphysema, lung fibrosis, cystic fibrosis, lung cancer, pulmonary interstitial disease, and/or monitoring pre- and post-lung transplantation, pulmonary emboli), cerebrovascular disease (for example, stroke, TIA, microvascular dementia), and emboli (such as pulmonary emboli, deep venous thrombosis), inadequate ventilation/oxygenation of a mechanically ventilated subject, inflammatory disease, auto-immune disease (with an ischemic component) or gastrointestinal disorders (for example (but not limited to) Crohn's disease, colitis ulcerosa), neurodegenerative disease (for example (but not limited to) Parkinson's disease or multiple sclerosis), erectile dysfunction, sport injury, frailty, shock, post-reanimation, and post-resuscitation, or a combination of two or more of the listed items. In some embodiments, the condition comprising perfusion shortage comprises a diseases or processes related to vascular remodeling including, but not limited to, arterial and venous diseases, as well as diseases related to lymphangiogenic disease, or vascular remodeling including (but not limited to) lymphangiogenic metastatic disease, arteriovenous malformation, arterial remodeling, and/or venous thrombosis, and/or placental development (e.g., to monitor menstrual cycle). In some embodiments, the disease or condition comprising perfusion shortage comprises (but not limited to) ischemic cardiovascular disease, microangiopathy in cardiovascular disease (such as diastolic heart failure), hypertrophic cardiomyopathy, cardiovascular pathology with relative ischemia (such as hypertrophic CMP, ischemic heart failure, and/or congenital cardiac disease), ischemic cerebrovascular disease (including CVA/TIA, and/or vascular dementia), ischemic peripheral artery disease, venous disease, orthopedic disease, sepsis, inflammation, lymphatic disease, ophthalmological disease associated with ischemia and/or neoangiogenesis (such as neoangiogenesis in retinopathy), diabetic macro and microangiopathy, oncological pathology (including solid tumors, metastatic disease), endothelial dysfunction in cardiovascular and peripheral artery disease, ophthalmology (such as neoangiogenesis in retinopathy), diabetic macro and microangiopathy, oncological pathology (such as solid tumors and/or metastatic disease), endothelial dysfunction in cardiovascular and peripheral artery disease, nephrological disease associated with ischemia and/or neoangiogenesis, gynecological disease, orthopedic disease, lung disease (such as COPD, emphysema, lung fibrosis, cystic fibrosis, lung cancer, pulmonary interstitial disease, and/or monitoring pre- and post-lung transplantation, pulmonary emboli), cerebrovascular disease (for example, stroke, TIA, microvascular dementia), and emboli (such as pulmonary emboli, deep venous thrombosis), inadequate ventilation/oxygenation of a mechanically ventilated subject, inflammatory disease and/or auto-immune disease (with an ischemic component) or gastrointestinal disorder (including (but not limited to) Crohn's disease, Colitis Ulcerosa), neurodegenerative disease (including (but not limited to) Parkinson's disease or multiple sclerosis), erectile dysfunction, sport injury, frailty, shock (such as post-resuscitation shock), arterial and venous diseases, disease related to lymphangiogenic disease, vascular remodeling including lymphangiogenic metastatic disease, arteriovenous malformation, arterial remodeling, or venous thrombosis, or a combination of two or more of any of the listed items.

In the method of some embodiments, the condition comprising perfusion shortage is selected from the group consisting of ischemic cardiovascular disease, microangiopathy in cardiovascular disease (such as diastolic heart failure), hypertrophic cardiomyopathy, cardiovascular pathology with relative ischemia (such as hypertrophic CMP, ischemic heart failure, and/or congenital cardiac disease), ischemic cerebrovascular disease (including CVA/TIA, and/or vascular dementia), ischemic peripheral artery disease, venous disease, orthopedic disease, sepsis, inflammation, lymphatic disease, ophthalmological disease associated with ischemia and/or neoangiogenesis (including (but not limited to) neoangiogenesis in retinopathy), diabetic macro and microangiopathy, oncological pathology (including solid tumors, metastatic disease), endothelial dysfunction in cardiovascular and peripheral artery disease, ophthalmology (such as neoangiogenesis in retinopathy), diabetic macro and microangiopathy, oncological pathology (including (but not limited) solid tumors and/or metastatic disease), endothelial dysfunction in cardiovascular and peripheral artery disease, nephrological disease associated with ischemia and/or neoangiogenesis, gynecological disease, orthopedic disease, lung disease (including (but not limited to) COPD, emphysema, lung fibrosis, cystic fibrosis, lung cancer, pulmonary interstitial disease, and/or monitoring pre- and post-lung transplantation), pulmonary emboli or deep venous thrombosis), cerebrovascular disease (including (but not limited to), stroke, TIA, microvascular dementia, and emboli such as pulmonary emboli), deep venous thrombosis, inadequate ventilation/oxygenation of a mechanically ventilated subject, inflammatory disease and/or auto-immune disease (with an ischemic component), gastrointestinal disorders (including (but not limited) Crohn's disease, colitis ulcerosa), neurodegenerative disease (including (but not limited) Parkinson's disease or multiple sclerosis), erectile dysfunction, sport injury, frailty, shock (such as post-resuscitation shock), arterial and venous diseases, disease related to lymphangiogenic disease, vascular remodeling including lymphangiogenic metastatic disease, arteriovenous malformation, arterial remodeling, or venous thrombosis, or a combination of two or more of the listed items.

In some embodiments, the condition comprising perfusion shortage comprises cardiovascular pathology with relative or absolute ischemia comprises congenital disease with significant or persistent shunting, such as open foramen ovale, ductus botalli, and/or ventricular shunt, or Eisenmenger syndrome/disease.

In some embodiments, the condition comprising perfusion shortage comprises relative or absolute ischemia. Without being limited by theory, ischemia can refer to decreased supply of oxygen and/or increased demand for oxygen. In some embodiments, ischemia due to increased demand for oxygen may occur due to, for instance, due to hypertrophy, inflammation or increased metabolism or sepsis

In some embodiments, the relative ischemia comprises cardiovascular disease with an increased demand for oxygen for instance but not limited to hypertrophic myocardium or and local and/or systemic increased oxygen demand (including but not limited to inflammation).

In some embodiments, a subject has at least one risk factor for having a condition comprising perfusion shortage, wherein said risk factor is selected from the group consisting of high blood pressure; glucose intolerance; family history of cardiovascular disease, hypercholesterolemia, or dyslipidemia; aging; smoking; physical inactivity; obesity; previous cardiovascular disease; diagnosis of diabetes mellitus, kidney disease, peripheral artery disease, or metabolic syndrome; gender; high low density lipoprotein (LDL) level; high cholesterol level; and specific DNA polymorphism associated with higher cardiovascular risk.

### Medical Monitoring and Intervention

In some embodiments, the method further comprises correlating the detected levels of the specified different gene products to a severity of the condition comprising perfusion shortage or a likelihood of developing the condition, thereby ascertaining a severity or risk of the condition. The correlating can be used to quantify the condition or the risk of the condition. By way of example, if the condition comprising perfusion shortage is present, or there is a risk of the condition, the subject can be recommended for a medical intervention or an invasive or resource-intensive monitoring as described herein (e.g., invasive angiography, advanced imaging, nuclear imaging, MSCT, MRI analysis, or tissue biopsy)) as described herein. Example medical interventions include, but are not limited to, initiation or modulation of pharmacotherapy or anti-ischemic medical intervention (including (but not limited to) percutaneous and/or surgical revascularization), and/or modulating the local (or general) perfusion and oxygenation. In some embodiments, the medical intervention is provided to the subject. In some embodiments, the subject receives the medical intervention. In some embodiments, the correlating can be performed after a medical intervention, for example to determine efficacy of the medical intervention, monitor disease progression, and/or to select further medical management or medical interventions.

In some embodiments, the method further comprises identifying a previous condition comprising perfusion shortage (such as an ischemic or neoangiogenesis episode), diagnosing the subject as having the condition, determining the subject to be at risk of the condition, prognosticating an outcome in the subject, predicting a response of the subject to a medical intervention or pharmacotherapy, selecting a medical intervention for the subject (for example, by way of a surrogate marker), selecting a dose and/or frequency of a medical intervention, validating a candidate medical intervention, measuring a response to a candidate medical intervention, measuring and monitoring a subject's ischemic disease progression or remission, and/or monitoring a subject's response to a medical intervention or pharmacotherapy based on the detected levels of the specified different gene products. For example, levels of the specified gene products outside of (e.g., exceeding the upper limit of) the control ranges can indicate that the subject has the condition, or is at risk for the condition.

In some embodiments, the method comprises recommending the subject for an anti- or pro-ischemic medical intervention and/or anti- or pro-neoangiogenesis medical intervention or the modification of the therapy when the expression levels of the specified different gene products are outside of the control ranges. Any suitable surgical and/or percutaneous procedure to treat a condition comprising ischemia and/or neoangiogenesis is contemplated. The medical intervention can be one that is approved by, and/or meets guidelines set by a local health specialist organisation, or by a professional or government organization, for example the Food and Drug Administration, or the European Medicines Agency.

In some embodiments, the method comprises providing an assessment of a severity of said subject's perfusion shortage, based on the output. In some embodiments, the output further comprises providing a prognosis report of said subject's perfusion shortage. In some embodiments, providing a prognosis report may be based on the determination of expression levels of two or more genes (nucleotide or protein biomarkers) from a plurality of the identified genes (nucleotide or protein biomarkers) described herein. In some embodiments, providing a prognosis report may be based on the determination of expression levels of two or more genes (nucleotide or protein biomarkers) from a plurality of the identified genes (nucleotide or protein biomarkers) that are specific for a female in case the subject is a female. In some embodiments, providing a prognosis report may be based on the determination of expression levels of two or more genes (nucleotide or protein biomarkers) from a plurality of the identified genes (nucleotide or protein biomarkers) that are specific for a male in case the subject is a male. In some embodiments, providing a prognosis report may be based on the determination of expression levels of two or more genes (nucleotide or protein biomarkers) from a plurality of the identified genes (nucleotide or protein biomarkers) that are specific for a male or a female in case the subject is a male or a female respectively, depending on the degree of difference of the expression levels of said two or more genes (nucleotide or protein biomarkers) in the subject with respect to a control value.

In some embodiments, a control value is the respective value in a control for example, an individual of the same gender as the subject, without a cardiovascular risk of having ischemia. A control value may be a baseline value for the level of each of the two or more genes in the subject prior to a cardiovascular event in the subject.

In some embodiments, said output may further comprise providing a response report of said subject upon, during, and/or after a medical intervention or modulation of pharmacotherapy. In some embodiments, said output may further comprise providing a monitoring report. The monitoring report may comprise monitoring the subject before or after receiving a medical intervention or modulation of pharmacotherapy. In some embodiments, the monitoring may occur periodically from the date of obtaining the samples to generate the output, and continue for a variable period of time, or as determined by a medical practitioner. In some embodiments, the monitoring may occur periodically for 2 months or more, for 4 months or more for 6 months or more, for 8 months or more, for 10 months or more, for 12 months or more for 14 months or more, for 16 months or more, for 18 months or more, for 20 months or more, for 22 months or more, or for 24 months, or more from the time the date of obtaining the samples to generate the output. In some embodiments, the monitoring may occur periodically for 36 months or more. In some embodiments, the monitoring may occur periodically for 48 months or more.

In some embodiments, the monitoring may occur once every 2 weeks, or once every 3 weeks, or once every 4 weeks, or once every 6 weeks, or once every 8 weeks, or once every 10 weeks, or once every 12 weeks, or once every 14 weeks, or once every 16 weeks, or once every 18 weeks, or once every 20 weeks, or once every 22 weeks, or once every 24 weeks.

In some embodiments, the method further comprises providing a treatment to said subject, wherein said subject has or is at risk of having a condition comprising perfusion shortage, at least in part based on said output. In some embodiments, providing a treatment comprises administering an anti- or pro-ischemic medical intervention and/or anti- or pro-neoangiogenesis medical intervention, and/or additional diagnostic analysis (e.g., biopsy) to the subject (or recommending administering an anti- or pro-ischemic medical intervention and/or anti- or pro-neoangiogenesis medical intervention, and/or additional diagnostic analysis (e.g., biopsy) to the subject) when the expression levels of the specified different gene products are outside of the control ranges. For example, in some embodiments, if the expression levels of the specified different gene products exceed the control ranges, and an anti-ischemic medical intervention or anti-neoangiogenesis medical intervention is provided.

In the method of some embodiments, the medical intervention comprises pharmacotherapy, radiotherapy or a surgical or percutaneous procedure, or a combination of two or more of the listed items. In the method of some embodiments, the medical intervention targets one or more of the at least two specified different gene products. In the method of some embodiments, the medical intervention or pharmacotherapy comprises (but is not limited to) an anti-ischemic medical intervention, for example a beta-blocker, a calcium antagonist, nitrates, thrombolysis, thrombectomy, bypass surgery, or a percutaneous coronary procedure. In some embodiments, the medical intervention comprises administering a pharmaceutical composition including but not limited to beta-blockers, calcium channels antagonists, nitrates, aspirin, cholesterol-lowering compounds, angiotensin-converting enzyme (ACE) inhibitors, and ranolazine. In some embodiments, the medical intervention comprises administering a pharmaceutical composition selected from the group consisting of (but not limited to) beta-blockers, calcium channels antagonists, nitrates, aspirin, cholesterol-lowering compounds, angiotensin-converting enzyme (ACE) inhibitors, and ranolazine. In some embodiments, the medical intervention comprises (but not limited to) one or more of atenolol, nadolol, metoprolol, propranolol, carteolol, carvedilol, labetolol, oxprenolol, penbutolol, sotalol, timolol, benazepril, captopril, cilazapril, enalapril, cnalaprilat fosinopril, iiriidapril, lisinopril, perindopril, quinapril, ramipril, trandolapril, candesartan, eprosartan irbesartan, losartan, elmesartan, telmisartan, valsartan, aspirin, diclofenae; diflunisal, etodolac, fenoproferi, flurbiprofen, ibuprofen, indomethaein, ketoprofen; Ketorolac, meclofenamate, mefenamic acid, meloxicam, naburnetone, naproxen, oxaprozin, piroxicani, salsalate, sulindac, tolmetin, a COX-2 inhibitor (comprises celecoxib rofecoNib, etoricoxib, valdecoxib, pareeoxib, meloxicarn or luniracoxib), ranolazine, nitrates, such as isosorbide mononitrate, isosorbide dinitrate (ISDN), andarine, ethylestrenol, mesterolone, methandrostenolone, methenolone, methyltestosterone, oxandrolone, oxymetholone stanozolol, boldenone, hexoxymestrolum, methandrostenolone, methenolone enanthate, nandrolone decanoate, nandrolone phenproprionate, stanozolol, stenbolone, testosterone cypionate, testosterone enanthate, testosteron, testosterone nicotinate, therobolin, trenbolone, trenbolone, trophobolene, a natural oil or fatty acid, e.g., comprising an omega-3 fatty acid, a fish oil, a long-chain polyunsaturated fatty acid, an n-3 and/or n-6 highly unsaturated fatty acid, eicosapentaenoic acid (EPA), docosaltexaenoic acid (DHA), calcium channel blocker, such as Nifidipine, Amlodipine, Felodipine, Nieardipine, Nisoldipine, Verapamil, or a combination thereof in some embodiments, the pharmaceutical composition may be packaged for administration intravenously, topically, orally, by inhalation, by infusion, by injection, intraperitoneally, intramuscularly, subcutaneously, intra-aurally, for intra-articular administration, for intra-mammary administration, for topical administration or for absorption through epithelial or mucocutaneous linings.

In the method of some embodiments, the medical intervention comprises a modulator of nucleic acid or protein expression comprising at least one of nucleotide or peptide sequences listed in Tables 3 and 4. In some embodiments, the medical intervention can target one or more genes (nucleotide or protein targets) listed in Tables 1-6. In some embodiments, the medical intervention may comprise a protein, a small molecule or a nucleic acid that can modulate the expression of the target genes comprising one or more genes (nucleotide or protein targets) listed in Tables 1-3.

In the method of some embodiments, the medical intervention comprises modulating the function of a gene listed in Tables 1-6. For example, modulating can comprise inhibiting the function of the gene product or enhancing the function of the gene product. Inhibiting (or reducing) can comprise, for example, (but not limited to) inhibiting transcription or translation, destabilizing or degrading a nucleic acid encoding the gene product or the gene product itself, changing the location of a nucleic acid encoding the gene product or the gene product itself, or forming a complex with the gene product. Enhancing (or increasing) can comprise, for example (but not limited to), inducing or increasing transcription or translation, stabilizing a nucleic acid encoding the gene product or the gene product itself, changing the location of a nucleic acid encoding the gene product or the gene product itself, or forming a complex with the gene product.

In some cases, the medical intervention comprises using nucleotides or peptides listed in Tables 3 and 4 or nucleotides or peptides derived from genes listed in Tables 1-6 as therapeutic targets. Suitable technologies may be used to direct to these therapeutic targets to modulate gene functions.

In some embodiments, the method further comprises ceasing or modulating the medical intervention and/or monitoring (or recommending ceasing or modulating the medical intervention and/or monitoring) when the expression levels of the specified different gene products are within the control ranges. For example, modulating can comprise adaptation of dose or frequency, or change of pharmacotherapy/intervention. It is contemplated that some kinds of patient monitoring can be invasive and/or resource-intensive (which, for shorthand, may collectively be referred to as “invasive monitoring”). Therefore, it can be advantageous to use methods, compositions, kits and uses as described herein to determine that the subject is at-risk before performing more invasive or resource-intensive analysis. In the method, composition, kit, or use of some embodiments, the invasive or resource-intensive monitoring comprises (but is not limited to) invasive angiography, advanced imaging, nuclear imaging, MSCT, MRI analysis, tissue biopsy, or monitoring of mechanical ventilation. Accordingly, in some embodiments, the method comprises determining the subject to be at risk for the condition, and further comprises invasive or resource-intensive monitoring after the subject has been determined to be at-risk. For example, in some embodiments, monitoring is performed for adequate ventilation and oxygenation of a mechanically ventilated patient, such as a patient in the CCU, ICU, and/or during surgery. In the method, composition, kit, or use of some embodiments, detecting a condition comprising perfusion shortage (or risk thereof) as described herein can be used in combination with a therapy for a condition comprising perfusion shortage as for example, a companion diagnostic. By way of example, the companion diagnostic can be used as a “gatekeeper” assay for the initiation of therapy, for modulation of therapy (e.g., reduction or increase of dose and/or frequency), efficacy analysis, and to monitor disease progression and remission. Accordingly, in some embodiments, the method of detecting a condition comprising perfusion shortage (or risk thereof) comprises determining whether or not to initiation therapy (based on whether the subject has or is at risk of a condition comprising perfusion shortage), for modulation of therapy (e.g., reduction or increase of dose and/or frequency), efficacy analysis, and to monitor disease progression.

In some embodiments, the subject may be monitored for, in addition to the monitoring aspects contemplated in the preceding sections, a condition or an event related to any one or more of: a trauma, condition or disease comprising: a chronic Systemic inflammatory Response State (SIRS); chronic systemic inflammatory stress; burns, chronic obstructive pulmonary disease; congestive heart failure; chronic kidney disease; surgery; cancer (and cancer metastasis (hematologic or lymphogenic metastasis); sepsis; ageing; acute respiratory distress syndrome; acute lung injury; infection; a CNS disorder or injury; anemia; immunosuppression; insulin resistance; anorexia; anxiety; sleep disturbances; weakness; fatigue; gastrointestinal distress; sleep disturbances; wake disturbances; pain; listlessness; shortness of breath; lethargy; depression; malaise; or, a combination thereof. In one embodiment, the trauma, condition or disease comprises a maladaptive nutritional state secondary to the SIRS. In one embodiment, the maladaptive nutritional state comprises cachexia, and optionally the cachexia comprises cachexia secondary to cancer. In one embodiment, the CNS disorder comprises Parkinson's disease or Alzheimer's disease. In some embodiments, at least based in part on response report, prognosis report, or both, further comprising ceasing said medical intervention or pharmacotherapy.

### Kits

In some embodiments, a kit is described. The kit can comprise (i) at least two different nucleic acid probes that hybridize to a portion of a transcript comprising sequences selected from Tables 3 and 4 or a reverse complement thereof, in which each of the nucleic acid probes comprises a different detectable moiety; and/or (ii) at least two different antibodies (or protein ligands) that each binds specifically to a peptide, in which at least a portion of the peptide is encoded by a sequence selected from Tables 3 and 4. In some embodiments, the kit consists essentially of or consists of (i) at least 2, 3, 4, 5, 6, 7, 8, 9, 10, 11, 12, 13, 14, 15, 16, 17, 18, or 19 different nucleic acid probes that hybridize to a portion of a transcript comprising sequences selected from Tables 3 and 4, or (ii) at least 2, 3, 4, 5, 6, 7, 8, 9, 10, 11, 12, 13, 14, 15, 16, 17, 18, or 19 different antibodies (or protein ligands) that each binds specifically to a peptide, in which at least a portion of the peptide is encoded by a sequence selected from Tables 3 and 4. In some embodiments, the kit consists essentially of or consists of (i) at least 2, 3, 4, 5, 6, 7, 8, 9, 10, 11, 12, 13, 14, 15, 16, 17, 18, or 19, but no more than 60 different nucleic acid probes that hybridize to a portion of a transcript comprising sequences selected from Tables 3 and 4, or (ii) at least 2, 3, 4, 5, 6, 7, 8, 9, 10, 11, 12, 13, 14, 15, 16, 17, 18, or 19, but no more than 60 different antibodies (or protein ligands) that each binds specifically to a peptide, in which at least a portion of the peptide is encoded by a sequence selected from Tables 3 and 4. In some embodiments, the kit consists essentially of or consists of (i) at least 2, 3, 4, 5, 6, 7, 8, 9, 10, 11, 12, 13, 14, 15, 16, 17, 18, or 19, but no more than 40 different nucleic acid probes that hybridize to a portion of a transcript comprising sequences selected from Tables 3 and 4, or (ii) at least 2, 3, 4, 5, 6, 7, 8, 9, 10, 11, 12, 13, 14, 15, 16, 17, 18, or 19, but no more than 40 different antibodies (or protein ligands) that each binds specifically to a peptide, in which at least a portion of the peptide is encoded by a sequence selected from Tables 3 and 4. In some embodiments, the kit consists essentially of or consists of (i) at least 2, 3, 4, 5, 6, 7, 8, 9, 10, 11, 12, 13, 14, 15, 16, 17, 18, or 19, but no more than 20 different nucleic acid probes that hybridize to a portion of a transcript comprising sequences selected from Tables 3 and 4. As described herein, a probe may be part of a primer pair, may be for quantifying amplicons in nucleic acid amplification, and/or may be for capturing a nucleic acid on a substrate. In some embodiments, the kit consists essentially of or consists of (ii) at least 2, 3, 4, 5, 6, 7, 8, 9, 10, 11, 12, 13, 14, 15, 16, 17, 18, or 19, but no more than 20 different antibodies (or protein ligands) that each binds specifically to a peptide, in which at least a portion of the peptide is encoded by a sequence selected from Tables 3 and 4. In some embodiments, the kit consists essentially of or consists of (i) at least 2, 3, 4, 5, 6, 7, 8, 9, 10, 11, 12, 13, 14, 15, 16, 17, 18, or 19, but no more than 20 different nucleic acid probes that hybridize to a portion of a transcript comprising sequences selected from Tables 3 and 4, or (ii) at least 2, 3, 4, 5, 6, 7, 8, 9, 10, 11, 12, 13, 14, 15, 16, 17, 18, or 19, but no more than 20 different antibodies (or protein ligands) that each binds specifically to a peptide, in which at least a portion of the peptide is encoded by a sequence selected from Tables 3 and 4. In some embodiments, nucleic acid probes that hybridize to a portion of a transcript comprising sequences selected from Tables 3 and 4 represent at least 50% of the total unique nucleic acid probes in the kit, for example, at least 50%, 60%, 70%, 80%, 90%, 95% or 100% of the unique nucleic acid probes in the kit. In some embodiments nucleic acid probes that hybridize to a portion of a transcript comprising sequences selected from Tables 3 and 4 represent at least 50% of the total unique nucleic acid probes in the kit, for example, at least 50%, 60%, 70%, 80%, 90%, 95% or 100% of the unique nucleic acid probes in the kit. In some embodiments, antibodies (or protein ligands) that each bind specifically to a peptide, in which at least a portion of the peptide is encoded by a sequence selected from Tables 3 and 4, represent at least 50% of the total unique antibodies (or protein ligands) in the kit, for example, at least 50%, 60%, 70%, 80%, 90%, 95% or 100% of the unique antibodies (or protein ligands) in the kit. In some embodiments, the kit can be for use in any of the methods described herein.

In some embodiments, the kit comprises at least two different nucleic acid probes or primer pairs that hybridize to a portion of a transcript comprising sequences selected from Tables 3 and 4 or a reverse complement thereof. Each of the nucleic acid probes can comprise a different detectable moiety, for example a fluorophore, a quantum dot, a radiolabel, or an enzyme. In some embodiments, a probe of the kit is a primer of a primer pair as described herein.

In some embodiments, the kit comprises at least two different antibodies (or protein ligands) that each bind specifically to a peptide. At least a portion of the peptide is encoded by a sequence selected from Tables 3 and 4. Optionally, the antibodies (or protein ligands) can each bind to a sequence encoded by one of Tables 3 and 4.

In some embodiments, the kit comprises at least two different nucleic acid probes that each hybridize to a portion of a different transcript comprising a sequence selected from SEQ ID NOs: 1-5103, Genes Number 1-9280, or a reverse complement thereof. By way of example, the nucleic acid probes can be parts of primer pairs. By way of example, each of the nucleic acid probes can comprise a different detectable moiety such as a barcode or fluorophore. Optionally, at least two different antibodies (or protein ligands) that each binds specifically to a peptide, on which at least a portion of the peptide is encoded by a sequence selected from Tables 3 and 4.

In the kit of some embodiments, (a) the nucleic acid probes comprise at least 6 but no more than 12 nucleic acid probes that each hybridize to a portion of a transcript comprising sequences selected from Table 3 or a reverse complement thereof, wherein each of the different nucleic acid probes comprises a different detectable moiety, and/or (b) at least 14 but no more than 40 nucleic acid probes that each hybridize to a portion of a transcript comprising sequences selected from Table 4 or a reverse complement thereof, wherein each of the different nucleic acid probes comprises a different detectable moiety. In the kit of some embodiments, (c) the at least two different antibodies (or protein ligands) comprise at least 6, but no more than 12 antibodies (or protein ligands) that each binds specifically to a peptide, and at least a portion of the peptide can be encoded by a sequence selected from Table 3, and each of the antibodies (or protein ligands) can comprises a different detectable moiety, and/or (d) the at least two different antibodies (or protein ligands) comprise at least 14, but no more than 20 antibodies (or protein ligands) that each binds specifically to a peptide, and at least a portion of the peptide is encoded by a sequence selected from Table 4, and each of the antibodies (or protein ligands) comprises a different detectable moiety.

In the kit of some embodiments, the at least two different nucleic acid probes comprise at least 6 but no more than 12 nucleic acid probes that each hybridize to a portion of a transcript comprising sequences selected from Table 3 or a reverse complement thereof. Each of the different nucleic acid probes can comprise a different detectable moiety.

In the kit of some embodiments, the at least two different nucleic acid probes comprise at least 14 but no more than 20 nucleic acid probes that each hybridize to a portion of a transcript comprising sequences selected from Table 4 or a reverse complement thereof. Each of the different nucleic acid probes can comprises a different detectable moiety.

In the kit of some embodiments, the at least two different antibodies (or protein ligands) comprise at least 6, but no more than 12 antibodies (or protein ligands) that each binds specifically to a peptide, in which at least a portion of the peptide is encoded by a sequence selected from Table 3. Each of the antibodies (or protein ligands) can comprises a different detectable moiety.

In the kit of some embodiments, the at least two different antibodies (or protein ligands) comprise at least 14, but no more than 20 antibodies (or protein ligands) that each binds specifically to a peptide, wherein at least a portion of the peptide is encoded by a sequence selected from Table 4. Each of the antibodies (or protein ligands) can comprises a different detectable moiety.

In some embodiments, the kit further comprises (i) a primer pair configured to amplify a transcript comprising a sequence from Table 3, in which at least one of the nucleic acid probes hybridizes to the transcript or portion thereof; or (ii) a primer pair configured to amplify a transcript comprising a sequence from Table 4, in which at least one of the nucleic acid probes hybridizes to the transcript or portion thereof. For example, the kit can comprise at least 2, 3, 4, 5, 6, 7, 8, 9, 10, 11, 12, 13, 14, 15, 16, 17, 18, or 19, but no more than 40 different primer pairs that each amplify a different transcript comprising a sequence from Table 3, such as 6-12 primer pairs, or at least 2, 3, 4, 5, 6, 7, 8, 9, 10, 11, 12, 13, 14, 15, 16, 17, 18, or 19, but no more than 40 different primer pairs that each amplify a different transcript comprising a sequence from Table 3, such as 14-20 primer pairs.

In some embodiments, the kit further comprises a primer pair configured to amplify a transcript comprising a sequence from Table 3. At least one of the nucleic acid probes as described herein can hybridize to the transcript or portion thereof. For example, the kit can comprise at least 2, 3, 4, 5, 6, 7, 8, 9, 10, 11, 12, 13, 14, 15, 16, 17, 18, or 19, but no more than 40 different primer pairs that each amplify a different transcript comprising a sequence from Table 3, such as 6-12 primer pairs.

In some embodiments, the kit further comprises a primer pair configured to amplify a transcript comprising a sequence from Table 4. At least one of the nucleic acid probes hybridizes to the transcript or portion thereof. For example, the kit can comprise at least 2, 3, 4, 5, 6, 7, 8, 9, 10, 11, 12, 13, 14, 15, 16, 17, 18, or 19, but no more than 40 different primer pairs that each amplify a different transcript comprising a sequence from Table 3, such as 14-20 primer pairs.

In the kit of some embodiments, the antibodies (or protein ligands) are each immobilized on a separate substrate, each substrate comprising a detectable moiety unique to the antibody immobilized thereon.

In the kit some embodiments, the kit further comprises an antibody that binds specifically to Flk1, Flt1, Flt4, or any other cell surface marker disclosed herein, or a combination of the listed items, optionally wherein the antibody is immobilized on a substrate. This antibody can be used to isolate Flk1+ cells or other hematopoietic EPCs with other cell surface marker(s) disclosed herein.

In the kit of some embodiments, the detectable moiety comprises (but is not limited to) a fluorophore, a radiolabel, a nanoparticle (such as a metal nanoparticle), magnetic bead, an enzyme, a quantum dot, or a nucleic acid barcode.

In the kit of some embodiments, the kit further comprises a reference composition comprising nucleic acids of cells. At least 50% of the nucleic acids can be from mononuclear endothelial stem cells from a peripheral blood sample of a control individual who does not have a perfusion shortage (e.g., ischemia and/or neoangiogenesis).

In some embodiments, a method of detecting a condition comprising perfusion shortage (such as neoangiogenesis and/or ischemia), or risk thereof in a subject is performed using the reagents of any of the kits or compositions described herein.

### Methods of Inhibiting, Ameliorating, Delaying the Onset of, Reducing the Likelihood of, Treating, or Preventing a Condition Comprising Neoangiogenesis or Ischemia

In some embodiments, a method of inhibiting, ameliorating, delaying the onset of, reducing the likelihood of, treating, or preventing a condition comprising neoangiogenesis or ischemia in a subject in need thereof is provided. The method can comprise administering to the subject a medical intervention comprising an effective amount of a modulator nucleic acid comprising at least one SEQ ID NOs: 1-5103 or Genes Number 1-9280. Optionally, the medical intervention further comprises an additional pharmacotherapy, or a surgical or percutaneous procedure, or a combination of two or more of the listed items. The modulator nucleic acid can be administered by any suitable means, for example, oral, intravenous, intramuscular, subcutaneous, intra-arterial or the like. The modulator nucleic acid can be administered in an amount effect to inhibit, reduce, or prevent or even increase expression, initiate expression or mimic the expression of genes listed in Tables 1-6. It will be appreciated that any method of inhibiting, ameliorating, delaying the onset of, reducing the likelihood of, treating, or preventing a condition comprising neoangiogenesis or ischemia described herein may be performed in conjunction with a method of detecting a condition comprising neoangiogenesis or ischemia as described herein. For example, the condition can be detected, and then the inhibiting, ameliorating, delaying the onset of, reducing the likelihood of, treating, or preventing a condition comprising neoangiogenesis or ischemia can be performed. Conceptually, in some embodiments, the method comprises any method of reducing, inhibiting, or preventing the increase of the expression levels of any of the gene products comprising or encoded at least in part by any of genes listed in Tables 1-6. Without being limited by theory, although the nucleic acids of Tables 1-6 are overexpressed in conditions comprising perfusion (such as ischemia and/or neoangiogenesis), it is contemplated that the overexpression can represent a component of disease or disease progression, or can represent a response to disease progression. Accordingly, it will be appreciated that a “modulator nucleic acid” can decrease the expression of a gene product, for example if the modulator nucleic acid comprises an antisense RNA, small interfering RNA (siRNA), microRNA (miRNA), short hairpin RNA (shRNA), or CRISPR gRNA. It will also be appreciated that a modulator nucleic acid can increase the expression of a gene product, for example by encoding the gene product. Accordingly, in some embodiments, a modulator nucleic is comprised by an expression vector, such as a lentiviral vector, adenoviral vector, or adeno-associated viral vector. It will be appreciated that a modulator nucleic acid comprises sufficient sequence identity to a target nucleic acid to either enhance activity of the encoded gene product (for example by encoding additional gene product) or to inhibit the encoded gene product (for example, by hybridizing to a target nucleic acid encoding the gene product. Accordingly, a modulator nucleic acid can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 consecutive nucleotides of a target nucleic acid. In some embodiments, a modulator nucleic acid hybridizes to a target nucleic acid under physiological conditions.

In the method of some embodiments, (a) if the subject is a female, the modulator nucleic acid comprises at least 1, 2, 3, 4, 5, 10, 15, 20, 25, 30, 35, 40, 45, or 50 consecutive nucleotides of one or more of any of Table 3 or an antisense strand thereof (for example, 2-14, 2-15, 2-16, 2-17, 2-18, 2-19, 2-20, 2-40, 4-14, 4-15, 4-20, 4-25, 4-30, 4-35, 4-40, 5-10, 5-14, 5-15, 5-16, 5-17, 5-18, 5-19, 5-40, 6-14, 6-15, 6-20, 6-25, 6-30, 6-35, 6-40, 8-14, 8-15, 8-20, 8-25, 8-30, 8-35, 8-40, 9-10, 9-14, 9-15, 9-16, 9-20, 9-40, 10-14, 10-15, 10-20, 10-25, 10-30, 10-35, or 10-40 of sequences listed in Table 3), or (b) if the subject is a male, the modulator nucleic acid comprises at least 5, 10, 15, 20, 25, 35, 40, 45, or 50 consecutive nucleotides of one or more of any at least one of Tables 2, 4, and 6 or an antisense strand thereof to the subject (for example, 2-14, 2-15, 2-16, 2-17, 2-18, 2-19, 2-20, 2-4-14, 4-15, 4-20, 4-25, 4-30, 4-35, 4-40, 5-10, 5-14, 5-15, 5-16, 5-17, 5-18, 5-19, 5-20, 5-40, 6-14, 6-15, 6-20, 6-25, 6-30, 6-35, 6-40, 8-14, 8-15, 8-20, 8-25, 8-30, 8-35, 8-40, 9-10, 9-14, 9-15, 9-16, 9-9-40, 10-14, 10-15, 10-20, 10-25, 10-30, 10-35, or 10-40 of the listed SEQ ID NO:s). In some embodiments, the same numerical range of gene products is detected for male and female subjects. In some embodiments, the modulator nucleic acid enhances activity of the gene product. In some embodiments, the modulator nucleic acid inhibits activity of the gene product, for example, comprising an antisense RNA, small interfering RNA (siRNA), microRNA (miRNA), short hairpin RNA (shRNA), or CRISPR gRNA.

In the method of some embodiments, if the subject is a female, the modulator nucleic acid comprises at least one of sequences listed in Table 3 or an antisense strand thereof.

In the method of some embodiments, if the subject is a male, the modulator nucleic acid comprises at least one of sequences listed in Table 4 or an antisense strand thereof.

In some embodiments, a method of inhibiting, ameliorating, delaying the onset of, reducing the likelihood of, treating, or preventing a condition comprising perfusion shortage in a subject in need thereof is provided. In some embodiments, the method comprises obtaining a sample of a subject, and obtaining expression levels from the sample of the subject. The expression levels can be of at least two specified different gene products from Tables 1-6. The expression levels of the specified different gene products can be outside of control ranges of expression levels of the specified different gene products. Accordingly, the method can comprise administering a medical intervention for the condition to the subject.

In the method of some embodiments, the cellular secretion is selected from the group consisting of: cell lysate, blood, plasma, serum, stool, lymph, cerebrospinal fluid, saliva, sputum, tears, sweat, semen, transudate, urine, exudate, tissue biopsy, and synovial fluid. In the method of some embodiments, the cellular secretion is selected from the group consisting of: blood, plasma, serum, stool, lymph, cerebrospinal fluid, saliva, sputum, tears, sweat, semen, transudate, urine, exudate, tissue biopsy, and synovial fluid

In some embodiments, the method further comprises correlating the detected levels of the specified different gene products to a severity of the condition or a likelihood of developing the condition, thereby ascertaining a severity or risk of the condition.

In the method of some embodiments, administering the medical intervention to the subject when the expression levels of at least 2, 3, 4, 5, 6, 7, 8, 9, 10, 11, 12, 13, 14, 15, 16, 17, 18, 19, 21, 22, 23, 24, 25, 26, 27, 28 29, 30, 31, 32, 33, 34, 35, 36, 37, 38, 39, 40, 50, or 60 specified different gene products are outside of the control ranges, including ranges between any two of the listed values, for example, 2-14, 2-15, 2-16, 2-17, 2-18, 2-19, 2-20, 2-40, 20-50, 20-60, 4-14, 4-15, 4-20, 4-4-30, 4-35, 4-40, 4-50, 4-60, 5-10, 5-14, 5-15, 5-16, 5-17, 5-18, 5-19, 5-20, 5-40, 5-50, 5-60, 6-14, 6-15, 6-20, 6-25, 6-30, 6-35, 6-40, 6-50, 6-60, 8-14, 8-15, 8-20, 8-25, 8-30, 8-35, 8-40, 8-50, 8-60, 9-9-14, 9-15, 9-16, 9-20, 9-40, 9-50, 9-60, 10-14, 10-15, 10-20, 10-25, 10-30, 10-35, 10-40, 10-50, or 10-60 specified different gene products.

In some embodiments, (a) if the subject is female, the method comprises determining the subject to have the condition or a risk thereof in the subject when the expression levels of at least two specified different gene products that each comprise a sequence selected from Table 3 (or peptide sequence encoded therefrom) are outside of the control ranges, and (b) if the subject is male, the method comprises determining the subject to have the condition or a risk thereof in the subject when the expression levels of at least two specified different gene products that each comprise a sequence selected from Table 4 (or peptide sequence encoded therefrom) are outside of the control ranges.

In some embodiments, if the subject is female, the method comprises determining the subject to have the condition or a risk thereof in the subject when the expression levels of at least two specified different gene products that each comprise a sequence selected from Table 3 (or peptide sequence encoded therefrom) are outside of the control ranges.

In some embodiments, if the subject is male, the method comprises determining the subject to have the condition or a risk thereof in the subject when the expression levels of at least two specified different gene products that each comprise a sequence selected from Table 4 (or peptide sequence encoded therefrom) are outside of the control ranges.

In some embodiments, (a) if the subject is female, the method comprises determining the subject to have the condition or risk thereof in the subject when the expression levels of 2-14, 2-15, 2-16, 2-17, 2-18, 2-19, 2-20, 2-40, 20-50, 20-60, 4-14, 4-15, 4-20, 4-25, 4-30, 4-35, 4-4-50, 4-60, 5-10, 5-14, 5-15, 5-16, 5-17, 5-18, 5-19, 5-20, 5-40, 5-50, 5-60, 6-14, 6-15, 6-20, 6-25, 6-30, 6-35, 6-40, 6-50, 6-60, 8-14, 8-15, 8-20, 8-25, 8-30, 8-35, 8-40, 8-50, 8-60, 9-10, 9-14, 9-15, 9-16, 9-20, 9-40, 9-50, 9-60, 10-14, 10-15, 10-20, 10-25, 10-30, 10-35, 10-40, 10-50, or 10-60 specified different gene products that each comprise a sequence selected from Table 3 (or peptide sequence encoded therefrom) are outside of the control ranges, and (b) if the subject is male, the method comprises determining the subject to have the condition or a risk thereof in the subject when the expression levels of 2-14, 2-15, 2-16, 2-17, 2-18, 2-19, 2-20, 2-40, 4-14, 4-15, 4-20, 4-25, 4-30, 4-35, 4-40, 5-10, 5-14, 5-15, 5-16, 5-17, 5-18, 5-19, 5-20, 5-40, 6-14, 6-15, 6-20, 6-25, 6-30, 6-35, 6-40, 8-14, 8-15, 8-20, 8-25, 8-30, 8-35, 8-40, 9-10, 9-14, 9-15, 9-16, 9-20, 9-40, 10-14, 10-15, 10-20, 10-25, 10-35, or 10-40 specified different gene products that each comprise a sequence selected from Table 4 (or peptide sequence encoded therefrom) are outside of the control ranges. In some embodiments, (a) if the subject is female, the method comprises determining the subject to have the condition or risk thereof in the subject when the expression levels of 2-14, 2-15, 2-16, 2-17, 2-18, 2-19, 2-20, 2-40, 4-14, 4-15, 4-20, 4-25, 4-30, 4-35, 4-40, 5-10, 5-14, 5-15, 5-16, 5-17, 5-18, 5-19, 5-20, 6-14, 6-15, 6-20, 6-25, 6-30, 6-35, 6-40, 8-14, 8-15, 8-20, 8-25, 8-30, 8-35, 8-40, 9-10, 9-14, 9-9-16, 9-20, 9-40, 10-14, 10-15, 10-20, 10-25, 10-30, 10-35, or 10-40 specified different gene products that each comprise a sequence selected from Table 3 (or peptide sequence encoded therefrom) are outside of the control ranges, and (b) if the subject is male, the method comprises determining the subject to have the condition or a risk thereof in the subject when the expression levels of 2-14, 2-15, 2-16, 2-17, 2-18, 2-19, 2-20, 2-40, 4-14, 4-15, 4-20, 4-25, 4-30, 4-35, 4-40, 5-10, 5-14, 5-15, 5-16, 5-17, 5-18, 5-19, 5-20, 5-40, 6-14, 6-15, 6-20, 6-25, 6-30, 6-35, 6-40, 8-14, 8-15, 8-20, 8-25, 8-30, 8-35, 8-40, 9-10, 9-14, 9-15, 9-16, 9-20, 9-40, 10-14, 10-15, 10-20, 10-25, 10-30, 10-35, or 10-40 specified different gene products that each comprise a sequence selected from Table 4 (or peptide sequence encoded therefrom) are outside of the control ranges. In some embodiments, the same numerical range of gene products is utilized for male and female subjects.

In some embodiments, if the subject is female, the method comprises determining the subject to have the condition or risk thereof in the subject when the expression levels of 2-14, 2-15, 2-16, 2-17, 2-18, 2-19, 2-20, 2-40, 20-50, 20-60, 4-14, 4-15, 4-20, 4-25, 4-30, 4-35, 4-40, 4-50, 4-60, 5-14, 5-15, 5-16, 5-17, 5-18, 5-19, 5-20, 5-40, 5-50, 5-60, 6-14, 6-15, 6-20, 6-25, 6-30, 6-35, 6-6-50, 6-60, 8-14, 8-15, 8-20, 8-25, 8-30, 8-35, 8-40, 8-50, 8-60, 9-10, 9-14, 9-15, 9-16, 9-20, 9-40, 9-50, 9-60, 10-14, 10-15, 10-20, 10-25, 10-30, 10-35, 10-40, 10-50, or 10-60 specified different gene products (or fewer than 60, 40, 30, or 20 specified different gene products) that each comprise a sequence selected from Table 3 (or peptide sequence encoded therefrom) are outside of the control ranges.

In some embodiments, if the subject is male, the method comprises determining the subject to have the condition or a risk thereof in the subject when the expression levels of only 2-14, 2-2-16, 2-17, 2-18, 2-19, 2-20, 2-40, 4-14, 4-15, 4-20, 4-25, 4-30, 4-35, 4-40, 5-10, 5-14, 5-15, 5-16, 5-18, 5-19, 5-20, 5-40, 6-14, 6-15, 6-20, 6-25, 6-30, 6-35, 6-40, 8-14, 8-15, 8-20, 8-25, 8-30, 8-8-40, 9-10, 9-14, 9-15, 9-16, 9-20, 9-40, 10-14, 10-15, 10-20, 10-25, 10-30, 10-35, or 10-40 specified different gene products (or fewer than 40, 30, or 20 specified different gene products) that each comprise a sequence selected from Table 4 (or peptide sequence encoded therefrom) are outside of the control ranges.

In some embodiments, (a) if the subject is female, the method comprises detecting, from the sample, expression levels of only 10-40 or 10-60 specified different gene products that each comprise a sequence selected from Table 3 (or peptide sequence encoded therefrom), and (b) if the subject is male, the method comprises detecting, from the sample, expression levels of only 10-40 specified different gene products that each comprise a sequence selected from Table 4 (or peptide sequence encoded therefrom).

In some embodiments, if the subject is female, the method comprises detecting, from the sample, expression levels of only 2-14, 2-15, 2-16, 2-17, 2-18, 2-19, 2-20, 2-40, 20-50, 20-60, 4-14, 4-15, 4-20, 4-25, 4-30, 4-35, 4-40, 4-50, 4-60, 5-10, 5-14, 5-15, 5-16, 5-17, 5-18, 5-19, 5-20, 5-5-50, 5-60, 6-14, 6-15, 6-20, 6-25, 6-30, 6-35, 6-40, 6-50, 6-60, 8-14, 8-15, 8-20, 8-25, 8-30, 8-35, 8-40, 8-50, 8-60, 9-10, 9-14, 9-15, 9-16, 9-20, 9-40, 9-50, 9-60, 10-14, 10-15, 10-20, 10-25, 10-30, 10-10-40, 10-50, or 10-60 specified different gene products (or fewer than 60, 40, 30, or 20 specified different gene products) that each comprise a sequence selected from Table 3 (or peptide sequence encoded therefrom).

In some embodiments, if the subject is male, the method comprises detecting, from the sample, expression levels of only 2-14, 2-15, 2-16, 2-17, 2-18, 2-19, 2-20, 2-40, 4-14, 4-15, 4-20, 4-25, 4-30, 4-35, 4-40, 5-10, 5-14, 5-15, 5-16, 5-17, 5-18, 5-19, 5-20, 5-40, 6-14, 6-15, 6-20, 6-25, 6-6-35, 6-40, 8-14, 8-15, 8-20, 8-25, 8-30, 8-35, 8-40, 9-10, 9-14, 9-15, 9-16, 9-20, 9-40, 10-14, 10-10-20, 10-25, 10-30, 10-35, or 10-40 specified different gene products (or fewer than 40, 30, or 20 specified different gene products) that each comprise a sequence selected from Table 4 (or peptide sequence encoded therefrom).

In the method of some embodiments, the medical intervention further comprises pharmacotherapy, or a surgical or percutaneous procedure, or a combination of two or more of the listed items.

In the method of some embodiments, the medical intervention comprises an anti-ischemic therapy selected from the group consisting of for example (but not limited to) beta-blockers, calcium antagonists, long- or short-acting nitrates, Ranolazine, artery bypass surgery (surgical revascularization), or a percutaneous coronary or arterial procedure, or any medical intervention (including suitable surgical and/or percutaneous procedure) to treat perfusion shortage (such as ischemia and/or neoangiogenesis) approved by, and/or meets guidelines set by a local health specialist organisation, or by a professional or government organization, for example the Food and Drug Administration, or the European Medicines Agency.

In the method of some embodiments, the medical intervention comprises a modulator nucleic acid or a modulator protein comprising or targeting at least one of genes listed in Tables 1-6 or gene products as described herein.

In the method of some embodiments, (a) if the subject is a female, the therapy comprises an modulator nucleic acid or a modulator protein comprising or targeting at least one of genes listed in Tables 1, 3, and 5 or gene products to the subject, or (b) if the subject is a male, the therapy comprises an modulator nucleic acid or a modulator protein comprising or targeting at least one of genes listed in Tables 2, 4, 6 or gene products to the subject.

In the method of some embodiments, the modulator nucleic acid comprises at least one of an antisense RNA, small interfering RNA, or CRISPR gRNA. In the method of some embodiments, the modulator nucleic acid is encoded by an expression vector, for example a lentiviral, adenoviral, or AAV vector for expressing the modulator nucleic acid in order to produce gene product encoded by the modulator nucleic acid (thus increasing activity of the gene product).

In the method of some embodiments, the subject may not present symptoms of any disease comprising perfusion shortage, such as neoangiogenesis and/or ischemia. For example, the subject may not present symptoms of any of ischemic cardiovascular disease, microangiopathy in cardiovascular disease (for example (but not limited to) diastolic heart failure), hypertrophic cardiomyopathy, cardiovascular pathology with relative ischemia (such as hypertrophic CMP, ischemic heart failure, and/or congenital cardiac disease), ischemic cerebrovascular disease (for example (but not limited to) CVA/TIA, and/or vascular dementia), ischemic peripheral artery disease, venous disease, orthopedic disease, sepsis, inflammation, lymphatic disease, ophthalmological disease associated with ischemia and/or neoangiogenesis (for example (but not limited to) neoangiogenesis in retinopathy), diabetic macro and microangiopathy, oncological pathology (for example (but not limited to) solid tumors, metastatic disease), endothelial dysfunction in cardiovascular and peripheral artery disease, ophthalmology (for example (but not limited to) neoangiogenesis in retinopathy), diabetic macro and microangiopathy, oncological pathology (for example (but not limited to) solid tumors and/or metastatic disease), endothelial dysfunction in cardiovascular and peripheral artery disease, nephrological disease associated with ischemia and/or neoangiogenesis, gynecological disease, orthopedic disease, lung disease (for example (but not limited to) COPD, emphysema, lung fibrosis, cystic fibrosis, lung cancer, pulmonary interstitial disease, and/or monitoring pre- and post-lung transplantation, pulmonary emboli), cerebrovascular disease (for example, stroke, TIA, microvascular dementia), and emboli (such as pulmonary emboli, deep venous thrombosis), inadequate ventilation/oxygenation of a mechanically ventilated subject, an inflammatory and auto-immune disease with an ischemic component, gastrointestinal disorders (for example (but not limited to) Crohn's disease, colitis ulcerosa), a neurodegenerative disease (for example (but not limited to) Parkinson's disease or multiple sclerosis), erectile dysfunction, sport injury, frailty, shock (such as post-resuscitation shock), arterial and venous diseases, disease related to lymphangiogenic disease, vascular remodeling including lymphangiogenic metastatic disease, arteriovenous malformation, arterial remodeling, or venous thrombosis, or a combination of two or more of the listed items. In the method of some embodiments, the subject may present symptoms of any disease comprising perfusion shortage as disclosed herein.

In the method of some embodiments, the condition comprising perfusion shortage is selected from the group consisting of: ischemic cardiovascular disease, microangiopathy in cardiovascular disease (such as diastolic heart failure), hypertrophic cardiomyopathy, cardiovascular pathology with relative ischemia (such as hypertrophic CMP, ischemic heart failure, and/or congenital cardiac disease), ischemic cerebrovascular disease (for example (but not limited to) CVA/TIA, and/or vascular dementia), ischemic peripheral artery disease, venous disease, orthopedic disease, sepsis, inflammation, lymphatic disease, ophthalmological disease associated with ischemia and/or neoangiogenesis (for example (but not limited to) neoangiogenesis in retinopathy), diabetic macro and microangiopathy, oncological pathology (for example (but not limited to) solid tumors, metastatic disease), endothelial dysfunction in cardiovascular and peripheral artery disease, ophthalmology (for example (but not limited to) as neoangiogenesis in retinopathy), diabetic macro and microangiopathy, oncological pathology (for example (but not limited to) as solid tumors and/or metastatic disease), endothelial dysfunction in cardiovascular and peripheral artery disease, nephrological disease associated with ischemia and/or neoangiogenesis, gynecological disease, orthopedic disease, lung disease (for example (but not limited to) COPD, emphysema, lung fibrosis, cystic fibrosis, lung cancer, pulmonary interstitial disease, and/or monitoring pre- and post-lung transplantation, pulmonary emboli), cerebrovascular disease (for example (but not limited to), stroke, TIA, microvascular dementia, and emboli (pulmonary emboli, deep venous thrombosis), inadequate ventilation/oxygenation of a mechanically ventilated subject, inflammatory disease and auto-immune disease with an ischemic component, gastrointestinal disorders (for example (but not limited to) Crohn's disease, colitis ulcerosa), a neurodegenerative disease (for example (but not limited to) Parkinson's disease or multiple sclerosis), erectile dysfunction, sport injury, frailty, shock (such as post-resuscitation shock), arterial and venous diseases, disease related to lymphangiogenic disease, vascular remodeling including lymphangiogenic metastatic disease, arteriovenous malformation, arterial remodeling, and venous thrombosis, or a combination of two or more of the listed items.

In the method of some embodiments, the condition comprising perfusion shortage comprises cardiovascular pathology with relative or absolute ischemia comprises congenital disease with significant or persistent shunting, such as open foramen ovale, ductus botalli, and/or ventricular shunt, or Eisenmenger syndrome/disease. Without being limited by theory, it is contemplated that the condition can comprise any congenital disease in which ischemia or neoangiogenesis plays an important role in the pathogenesis or determines prognostication or response to therapy.

In the method of some embodiments, the condition comprises relative or absolute ischemia. In some embodiments, ischemia due to increased demand for oxygen may occur due to, for instance, due to hypertrophy, inflammation or increased metabolism or sepsis.

In the method of some embodiments, the relative ischemia comprises cardiovascular disease with an increased demand for oxygen for instance but not limited to hypertrophic myocardium or and local and/or systemic increased oxygen demand (including but not limited to inflammation).

### Additional Embodiments

In some embodiments, a test to diagnose, measure, quantify and monitor disease comprising perfusion shortage (ischemic periods) in patients is provided. This test may also be used to measure therapy responsiveness in patients, for example anti-ischemic therapy, pro-ischemic therapy, measure neoangiogenesis, and increased tissue perfusion. Further, the test may be used to monitor disease progression or remission for better personalized and adjustable treatment in patients.

In some embodiments, disclosed herein is the identification of biomarkers that can identify and measure these episodes of oxygen deprivation with great certainty. Disclosed herein are unique diagnostic tests (method of blood processing), unique set of biomarkers, and a specific biostatistical algorithm and reference values to determine abnormal or pathological outcome/values in patients with disease comprising perfusion shortage such as neoangiogenesis (for example, tumor neoangiogenesis, and hematological metastatic disease, or ophthalmologic disease with retinal neoangiogenesis) or ischemia (for example cardiovascular or any other form of ischemic disease) from normal values in subjects without ischemic disease.

In some embodiments, molecular biomarkers have been identified that are up-regulated in patients having reperfusion shortage (such as ischemic patients) and may be used to diagnose (and quantify) ischemia including silent or transient ischemic episodes in CAD patients, and thus are likely to serve as sensitive biomarkers for the detection of mild ischemia in coronary, cerebrovascular and peripheral artery disease patients (and other ischemic patients). The gene expression patterns involved in the hypoxic response and/or the regulation of vasculogenesis likely represent a specific biomarker (RNA) profile that can be used for the diagnosis of coronary, cerebrovascular and peripheral artery disease.

The extent of reperfusion shortage (e.g., number and severity of ischemic episodes) may correlate with an incremental biological response and corresponding upregulation of the molecular biomarkers. Hence, the biomarkers may be used as to quantify ongoing ischemia in patients (using arbitrary units/scale), for example to quantify the extent of disease comprising perfusion shortage. Further, the biomarkers may also be used to monitor disease progression or remission over time.

The markers may be upregulated/responding rapidly to ischemic episodes, whereas other may be responding slowly to the hypoxic conditions. The former markers could be useful/appropriate in the dynamic setting of a hospital in which clinical decision making occurs from hour-to-hour (instantaneously), whereas the latter would be more useful in an outpatient setting (for a general practitioner or for population screening).

The methods (protocol, biomarkers, reference values and algorithm) of some embodiments disclosed herein may be used for the identification of current and past ischemic events (irrespective of its location (for instance (but not limited to) cerebral, myocardial, claudication) and can be used for the diagnosis of ischemic disease (including, but not limited to cardiovascular disease, cerebrovascular disease and peripheral artery disease).

The methods (protocol, biomarkers, reference values and algorithm) of some embodiments herein may be used for the identification of current and past ischemic events (irrespective of its location) and may be used for monitoring the progression of ischemic disease (including cardiovascular disease, cerebrovascular disease and peripheral artery disease), and to monitor the response (efficacy) to initiated therapy (pharmacotherapy or invasive therapy).

The methods (protocol, biomarkers, reference values and algorithm) of some embodiments herein may also be used to prognosticate/predict future outcome in patients with ischemic disease or disease with an important ischemic or neoangiogenesis component (mortality and adverse events) and to predict and measure/quantify the response of patients with ischemic disease (or disease with an important ischemic or neoangiogenesis component) to specific therapy (and therefore can be used to determine medical decision making (patient stratification and companion use of this assay for specific therapies or in depth diagnostic tracts (including more advanced imaging).

### EXAMPLES

The following examples are non-limiting and other variants within the scope of the art also contemplated.

### Example 1—Isolation of Flk1+(KDR1) Cells

KDR1 (also referred to as Flk1, VEGFR2, CD309 or Kdr) is a key marker defining hematopoietic stem cells. Human KDR1+ cells were isolated using immuno-selection by flow cytometry. After a venous blood draw, whole blood is processed by lysis of the erythrocytes, followed by a Ficoll gradient isolation of the mononucleated cells. The Flk1+(or Flt1+/Flt4+) cell preparation is isolated using an immunoseperation and a proprietary antibody directed against the CD cell surface markers Flk1+(CD309, KDR1, VEGFR2 or alternatively Flt1+/Flt4+). After cell isolation, the preparation is stored at −80° C. for further analysis, or processed for differential gene expression analysis or protein expression using DNAchip analysis, qPCR or any other method for differential expression analysis of nucleotide biomarkers.

### Example 2—Screen for Differentially Expressed Gene Products

A screen was performed for molecular biomarkers (here (RNA) oligonucleotides/candidate regulatory genes) using DNA chip analysis of 244 symptomatic patients with potential (cardiac/coronary) hypoxic episodes. Circulating KDR1-cells were isolated from blood samples, as these cells are involved in the physiological hypoxic response. Molecular expression patterns were cross-correlated with clinical diagnosis using current standard-of-care/best-of-care diagnostic tract. In order to increase the accuracy of the diagnoses in this clinical cohort (and reduce the number of false positive/false negative diagnoses), patients received a 6 month clinical telephone follow-up (and/or review of the clinical follow-up tract): in patients that were originally diagnosed as/allocated to the “angina pectoris excluded group” (no myocardial ischemia detectable), did hence not receive any (anti-ischemic) therapy as follow-up (and should not have shown any progression of ischemic heart disease (i.e. no persistent and/or progressive angina episodes and/or major cardiovascular events (including myocardial infarction). In the case a patient was inadvertently classified as “no myocardial ischemia detectable/diagnosed”, it should be possible to capture this misclassification (false negative) at 6-month clinical follow-up as the complaints would have deteriorated without appropriate pharmacotherapy or medical intervention. This is expected to reduce the number of false negative/inappropriately classified patients (patients with ischemia inadvertently diagnosed as “healthy”). Patients initially diagnosed as ischemic, with initiation of anti-ischemic pharmacotherapy or more invasive intervention should find confirmation of their diagnosis in their response to the specifically initiated pharmacotherapy, or upon invasive imaging and/or therapy (and their respective response to intervention). This should reduce the number of patients with false positive diagnosis. On average the number of initially falsely allocated patients using current standard-of-care diagnostics is estimated to be 16-26%.

Cross correlation of these clinical data with the molecular data set identified molecular markers (nucleotide probe or peptides) for female patients listed in Table 3 and molecular (nucleotide probe or peptides) markers for male patients that were statistically significantly differentially expressed in the classifier clinical data set. These markers and relative expression levels are shown in Tables 1-6. High throughput computing (i.e. machine learning) comparing different biostatistical analyses of the various combinations of biomarkers (out of the identified pool) showed that a minimal combination of 8 molecular biomarkers in female patients and a minimal combination of 12 biomarkers in male patients is sufficient to identify ischemic patients with the maximal/greatest accuracy in a separate independent patient cohort. This led to the misclassification error (alpha and beta error combined) in male patients of minimal 1.3% and in female patients of <0.05%. The misclassification rate of the current standard-of-care/best-of-care diagnostic tract involving multiple functional and imaging tests is 16-26%. The analysis of this assay comparing (individual) molecular biomarkers against reference values provided an objective (uniform) classification of the diagnosis of patient with a superior accuracy. The current standard-of-care/best-of-care diagnostic tract requires the (subjective) interpretation of exercise performances, ECG alterations and imaging phenomena by medical professionals (which is the basis of variability/inconsistency).

### Example 3—Analyses of Differential Expression of Gene Products in Female Patients

Analyses of differential expression of gene products in samples collected from ischemic female patients were performed using Affymetrix array and/or GeneChip Arrays. For female patients, the expression levels of genes listed in Tables 1, 3, 5 were up- or downregulated in ischemia (compared to non-ischemia controls). The current disclosure shows density plots of individual arrays and plots of individual arrays after frozen RNA normalization and showing re-analysis of 244 blood samples using Affymetrix Exon 2.0 microarrays. The differently expressed nucleic acids are shown in Tables 1, 3, 5. Accordingly, in accordance with some embodiments herein, it is shown that gene products corresponding to the genes listed in Tables 1, 3, 5 are expressed outside of control ranges in ischemic females.

### Example 4—Analyses of Differential Expression of Gene Products in Male Patients

Analyses of differential expression of gene products in samples collected from ischemic male patients were performed using Affymetrix Exon 2.0 array and/or GeneChip Arrays. For male patients, the genes listed in Tables 2, 4, 6 were up- or downregulated in ischemia (compared to non-ischemia controls). The current disclosure shows density plots of individual arrays and density plots of individual arrays after frozen RNA normalization and showing re-analysis of 244 blood samples using Affymetrix Exon 2.0 microarrays. The differently expressed nucleic acids are shown in Tables 2, 4, 6. Accordingly, in accordance with some embodiments herein, it is shown that gene products corresponding to the genes listed in Tables 2, 4, 6 are expressed outside of control ranges in ischemic males.

### Example 5—Validation Study for Female Patients

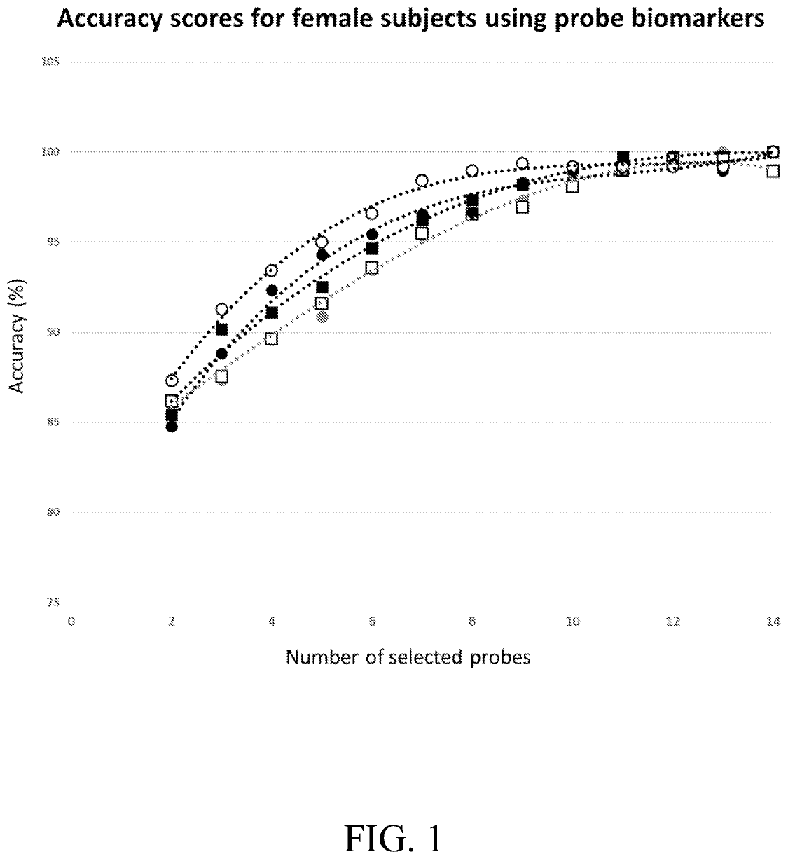

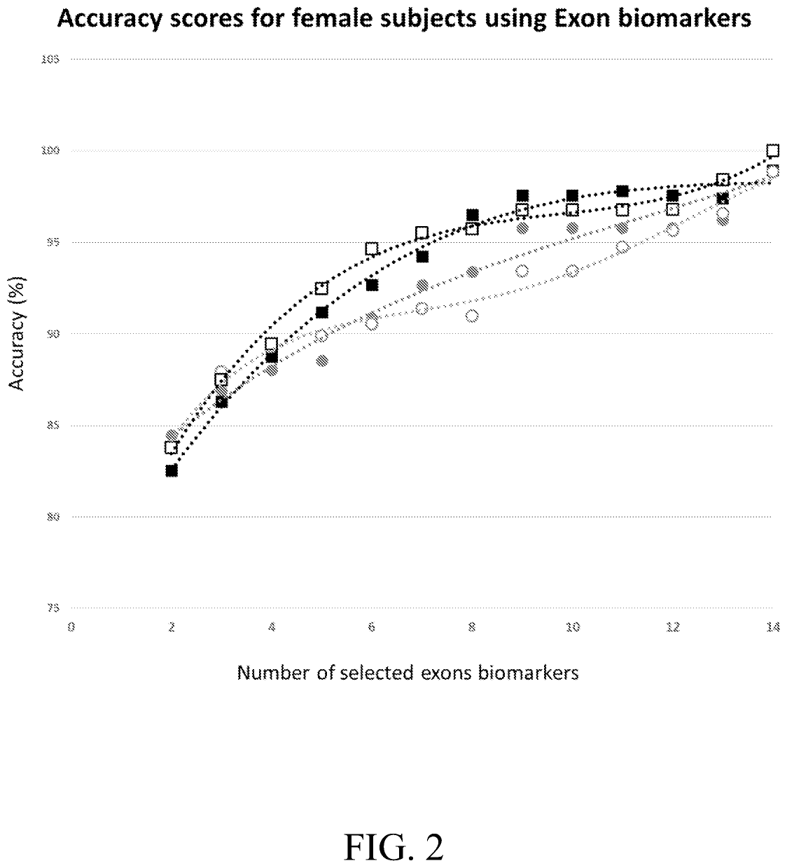

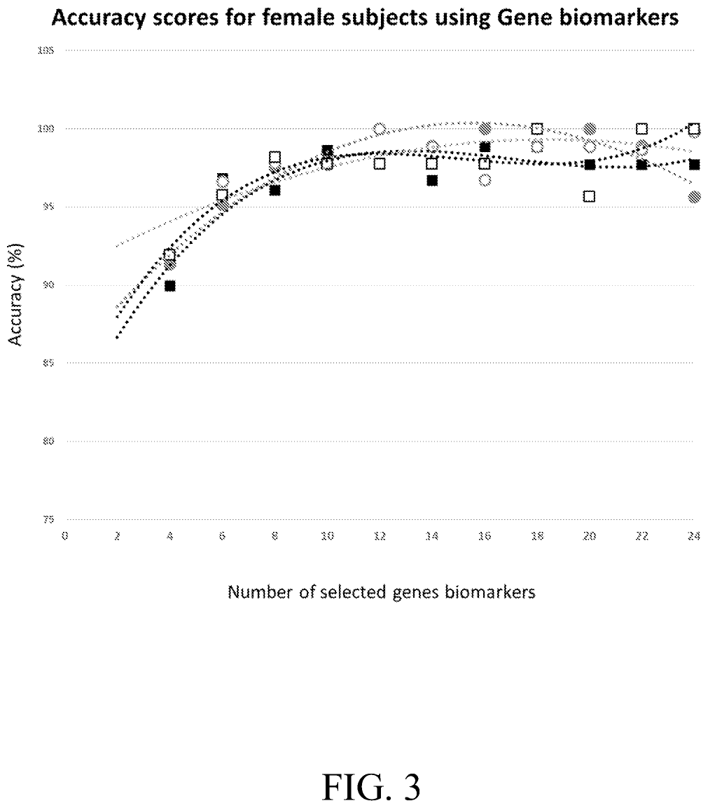

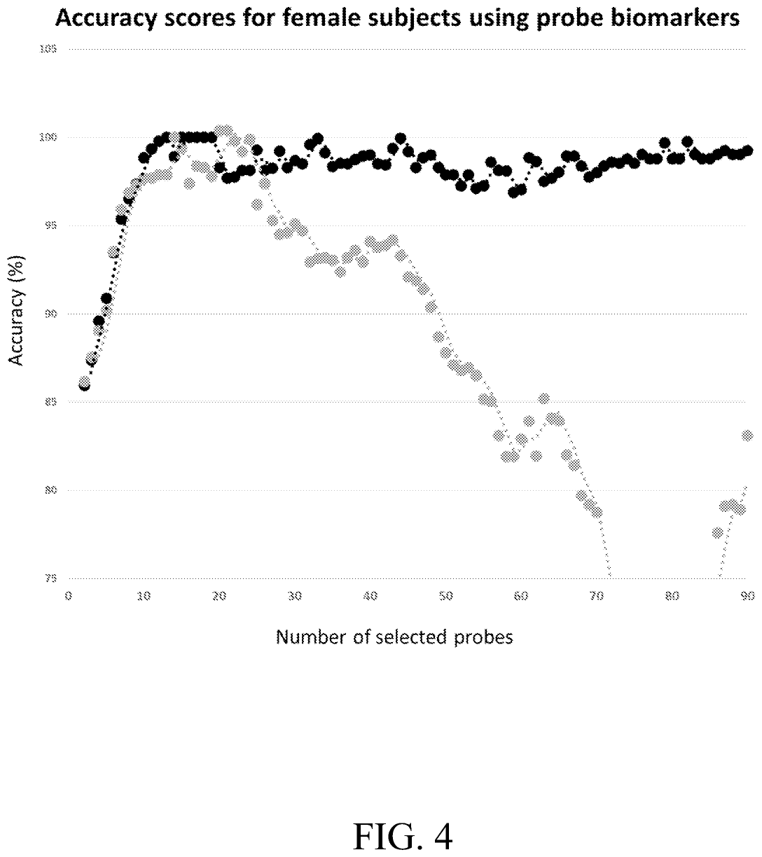

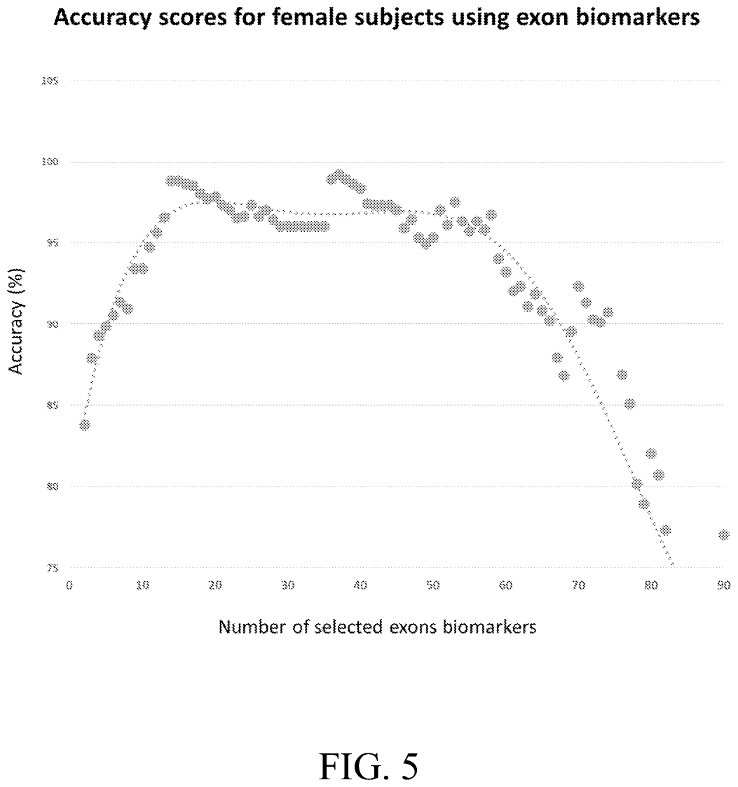

Validation of detection of conditions comprising ischemia and/or neoangiogenesis was performed for female patients using gene algorithms, exon algorithms and probe algorithms. Data in FIGS. 1-5 show that superior classification (as measured by classification scores) was observed for numbers of variables in Female biopanel ranging from 8-14 nucleotide biomarkers, resulting in an accuracy of >99.9% sensitivity using Fisher Discriminant analysis/Diagonal linear discriminant analysis. FIG. 1 shows a plot of classification scores with fitted curves for probes (gene products) for female patients using different statistical methods according to some embodiments. FIG. 2 shows a plot of classification scores with fitted curves for exons (gene products) for female patients using different statistical methods according to some embodiments. FIG. 3 shows LDA accuracy with fitted curve for exons versus the number of selected variables (gene products) for female patients according to some embodiments. FIGS. 4 and 5 show DLDA misclassification (FIG. 4) and LDA misclassification rate (FIG. 5) with fitted curves for probes versus the number of selected variables (gene products) for female patients according to some embodiments.

As shown by the data in FIGS. 1-5, superior classification (as indicated by high accuracy) was observed when the number of selected variables (number of gene products) for samples from female patients was around >8-10. The data were analyzed using several statistical methods. The data showed an accuracy of >99.9%. The data indicated that several statistical methods can be used for detection/diagnosis of conditions comprising ischemia in female patients with nearly 100% accuracy in accordance with some embodiments herein.

Table 8-Table 13 show data for accuracy of detection of ischemic heart disease in female patients. Variables indicated the number of gene products used in the method. The gene products were selected from Table 1, 3, 5.

TABLE 8

| | | | Accuracy |
|---|---|---|---|
| Probe Algorithm | No. of gene | type 1 + type 2 | (100% - type 1 and |
| Female subjects | products | error (%) | type 2 error (%)) |
| DLDA | 10 | 1.2 | 98.9 |
| FDA | 10 | 0.9 | 99.1 |
| LDA | 10 | 2.3 | 97.7 |
| PLSLDA | 10 | 1.9 | 98.1 |
| PLSRF | 10 | 1.0 | 99.0 |
| QDA | 10 | 0.8 | 99.2 |
| RF | 10 | 4.7 | 95.3 |

TABLE 9

| | | | Accuracy |
|---|---|---|---|
| Exon Algorithm | No. of gene | type 1 + type 2 | (100% - type 1 and |
| Female subjects | products | error (%) | type 2 error (%)) |
| DLDA | 10 | 4.2 | 95.8 |
| FDA | 10 | 2.4 | 97.6 |
| LDA | 10 | 6.6 | 93.4 |
| PLSLDA | 10 | 3.2 | 96.8 |
| PLSRF | 10 | 6.1 | 93.9 |
| QDA | 10 | 8.7 | 91.3 |
| RF | 10 | 8.9 | 91.1 |

TABLE 10

| | | | Accuracy |
|---|---|---|---|
| Gene Algorithm | No. of gene | type 1 + type 2 | (100% - type 1 and |
| Female subjects | products | error (%) | type 2 error (%)) |
| DLDA | 10 | 1.6 | 98.5 |
| FDA | 10 | 1.4 | 98.6 |
| LDA | 10 | 2.3 | 97.7 |
| PLSLDA | 10 | 2.3 | 97.8 |
| PLSRF | 10 | 3.9 | 96.1 |
| QDA | 10 | 4.6 | 95.4 |
| RF | 10 | 5.6 | 94.4 |

TABLE 11

| | | | Accuracy |
|---|---|---|---|
| Probe Algorithm | No. of gene | type 1 + type 2 | (100% - type 1 and |
| Female subjects | products | error (%) | type 2 error (%)) |
| DLDA | 14 | 1.1 | 98.9 |
| FDA | 14 | <0.05 | ^{~}100.0 |
| LDA | 14 | <0.05 | ^{~}100.0 |
| PLSLDA | 14 | 1.1 | 98.9 |
| PLSRF | 14 | <0.05 | ^{~}100.0 |
| QDA | 14 | <0.05 | ^{~}100.0 |
| RF | 14 | 4.3 | 95.7 |

TABLE 12

| | | | Accuracy |
|---|---|---|---|
| Exon Algorithm | No. of gene | type 1 + type 2 | (100% - type 1 and |
| Female subjects | products | error (%) | type 2 error (%)) |
| DLDA | 14 | <0.05 | ^{~}100.0 |
| FDA | 14 | 1.1 | 99.0 |
| LDA | 14 | 1.2 | 98.9 |
| PLSLDA | 14 | <0.05 | ^{~}100.0 |
| PLSRF | 14 | 1.1 | 98.9 |
| QDA | 14 | 4.1 | 95.9 |
| RF | 14 | 6.4 | 93.6 |

TABLE 13

| | | | Accuracy |
|---|---|---|---|
| Gene Algorithm | No. of gene | type 1 + type 2 | (100% - type 1 and |
| Female subjects | products | error (%) | type 2 error (%)) |
| DLDA | 14 | 1.1 | ^{~}98.9 |
| FDA | 14 | 3.3 | 96.7 |
| LDA | 14 | 1.2 | 98.9 |
| PLSLDA | 14 | 2.3 | 97.8 |
| PLSRF | 14 | <0.05 | ^{~}100 |
| QDA | 14 | 10.5 | 89.5 |
| RF | 14 | 5.4 | 94.6 |

### Example 6—Validation Study for Male Patients

Validation of detection of conditions comprising (cardiac) ischemia was performed for male patients using exon, probe, and gene algorithms.

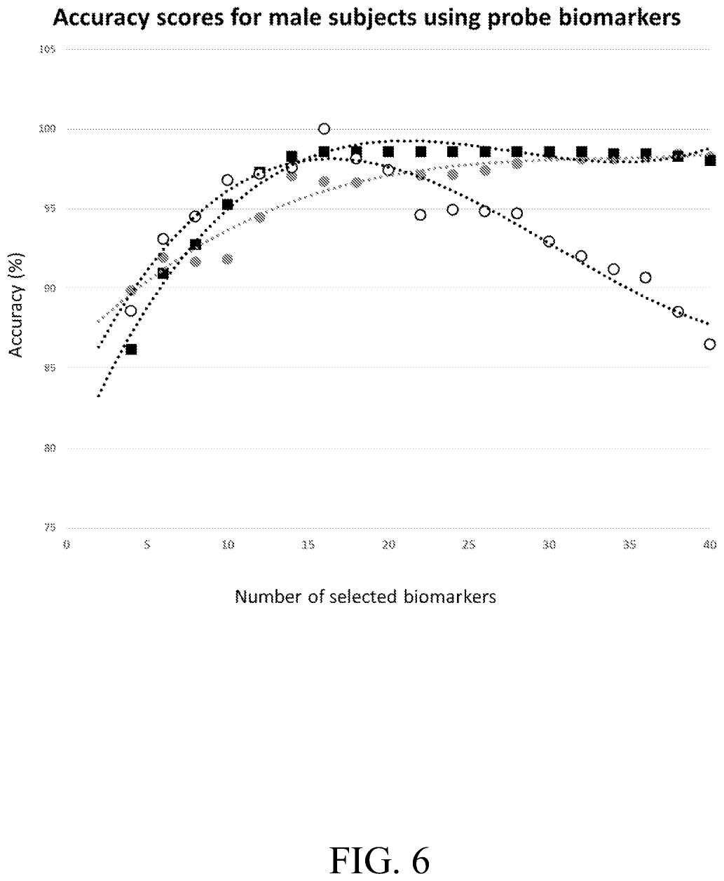

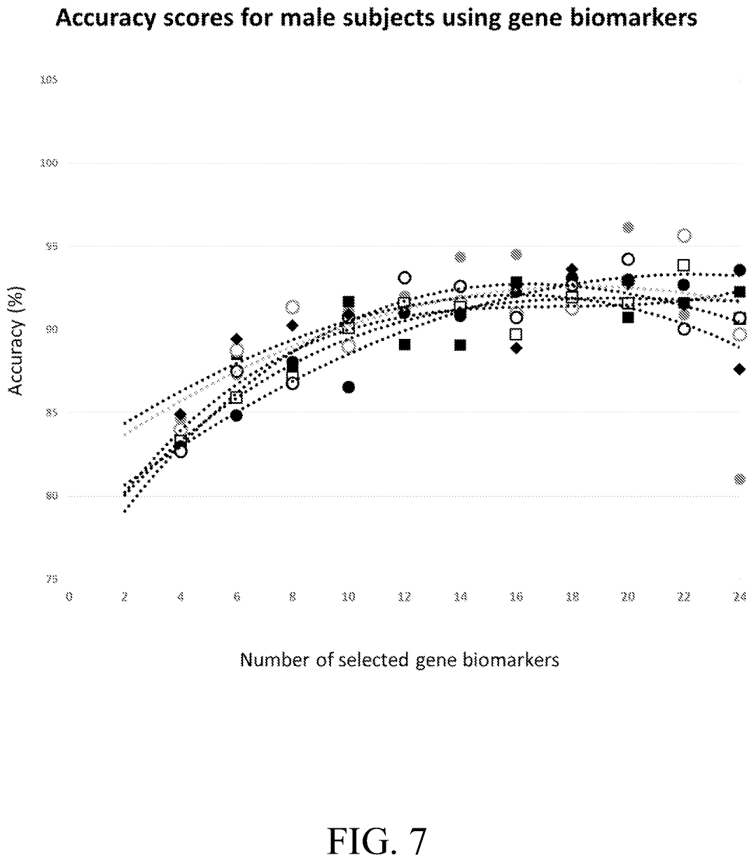

Data in FIGS. 6 and 7 show that superior classification (as measured by classification scores) was observed for numbers of variables in male biopanel >15, resulting in a misclassification of 1.7% (or >98.3% sensitivity) using Fisher Discriminant analysis/Diagonal linear discriminant analysis. FIG. 6 shows a plot of classification scores with fitted curves for probes for male patients according to some embodiments. FIG. 7 shows plots of classification scores with fitted curves for genes for male patients according to some embodiments. Accuracy as a measure of how well this binary classification test correctly identifies or excludes a condition, here ischemic heart disease, is defined as the proportion of correct predictions (both true positives and true negatives) among the total number of cases examined or 100%−alpha and beta error. As shown by the data in FIGS. 6 and 7, the optimal number of selected variables (number of gene products) for samples from male patients was >14. The data were analyzed using several statistical methods. The data showed an accuracy of 98.3%. The data indicated that several statistical methods can be used for detection/diagnosis of conditions comprising ischemia in male patients with nearly 100% accuracy in accordance with some embodiments herein.

Tables 14-16 show data for accuracy of detection of ischemic heart disease in male patients. Variables indicated the number of gene products used in the method. The gene products were selected from Tables 2, 4, 6.

TABLE 14

| | | | Accuracy |
|---|---|---|---|
| Probe Algorithm | No. of gene | type 1 + type 2 | (100% - type 1 and |
| Male subjects | products | error (%) | type 2 error (%)) |
| DLDA | 14 | 2.9 | 97.1 |
| FDA | 14 | 1.7 | 98.3 |
| LDA | 14 | 2.4 | 97.6 |
| PLSLDA | 14 | 11.3 | 88.7 |
| PLSRF | 14 | 7.2 | 92.8 |
| QDA | 14 | 2.4 | 97.6 |
| RF | 14 | <0.05 | ^{~}100 |

TABLE 15

| | | | Accuracy |
|---|---|---|---|
| Exon Algorithm | No. of gene | type 1 + type 2 | (100% - type 1 and |
| Male subjects | products | error (%) | type 2 error (%)) |
| DLDA | 14 | 6.5 | 93.5 |
| FDA | 14 | 3.5 | 96.5 |
| LDA | 14 | 7.8 | 92.2 |
| PLSLDA | 14 | 2.9 | 97.1 |
| PLSRF | 14 | 8.3 | 91.7 |
| QDA | 14 | 7.2 | 92.8 |
| RF | 14 | 6.4 | 93.6 |

TABLE 16

| | | | Accuracy |
|---|---|---|---|
| Genes Algorithm | No. of gene | type 1 + type 2 | (100% - type 1 and |
| Male subjects | products | error (%) | type 2 error (%)) |
| DLDA | 14 | 5.6 | 94.4 |
| FDA | 14 | 10.9 | 89.1 |
| LDA | 14 | 8.3 | 91.7 |
| PLSLDA | 14 | 8.6 | 91.4 |
| PLSRF | 14 | 9.2 | 90.8 |
| QDA | 14 | 7.4 | 92.6 |
| RF | 14 | 9.0 | 91.0 |

### Example 7—Validation Study for Male and Female Patients

Validation of detection of conditions comprising ischemia/neoangiogenesis was performed for male and female patients using gene algorithms, exon algorithms, and probe algorithms.

Tables 17 and Table 18 show data for accuracy of detection of ischemic heart disease in male and female patients combined. Variables indicated the number of gene products used in the method. The gene products were selected from Tables 1-6. Depending on the statistical method used, there was an accuracy of 67-75%. These data show that ischemic heart disease in both male and female patients can be accurately detected by methods in accordance with some embodiments herein, with an accuracy of 67% of higher.

TABLE 17

| | | | Accuracy |
|---|---|---|---|
| | No. of gene | type 1 + type 2 | (100% - type 1 and |
| Exon Algorithm | products | error (%) | type 2 error (%)) |
| DLDA | 24 | 33.0 | 67.0 |
| FDA | 24 | 33.5 | 66.5 |
| LDA | 24 | 30.5 | 69.5 |
| PLSLDA | 24 | 27.0 | 73.0 |
| PLSRF | 24 | 31.1 | 68.9 |
| QDA | 24 | 33.5 | 66.5 |
| RF | 24 | 26.7 | 73.3 |

TABLE 18

| | | | Accuracy |
|---|---|---|---|
| | No. of gene | type 1 + type 2 | (100% - type 1 and |
| Probe Algorithm | products | error (%) | type 2 error (%)) |
| DLDA | 20 | 27.2 | 72.8 |
| FDA | 20 | 24.8 | 75.2 |
| LDA | 20 | 24.7 | 75.3 |
| PLSLDA | 20 | 26.7 | 73.3 |
| PLSRF | 20 | 29.3 | 70.7 |
| QDA | 20 | 25.4 | 74.4 |
| RF | 20 | 26.2 | 73.8 |

### Example 8—Comparison to MIBI-SPECT Tracking

Blood samples of patients with anginal complaints and scheduled for elective nuclear and CMR (cardiac magnetic resonance imaging) perfusion imaging (MIBI SPECT) are collected. MIBI-SPECT is tracking of radionuclide-labelled molecules to the well-perfused myocardial tissue target tissues and is generally considered to be the gold standard for determination of imaging in the clinic. Initial diagnosis and long-term clinical outcome of these patients are prospectively cross-correlated with expression patterns of molecular biomarkers comprising or encoded by SEQ ID NOs: 1-5103 (or a peptide sequence encoded therefrom), Genes Number 1-9280 (or a peptide sequence encoded therefrom). Cross correlations are made with diagnosis, progression (and prognosis (MACCE events)) and therapy responsiveness.

### Example 9—Accuracy of Diagnosis by Proposed MyCor Classifier Methods

In the first analysis of 244 patients, the proof of concept was verified. Data indicated very high rate of correct diagnosis, i.e., in 98.3-99.9% of the patients (without the need for patient selection). The number of false negative and false positives was extremely low, i.e., <0.05-1.7%.

The current standard-of-care diagnostic tract of ischemic heart disease requires ECG, blood work, exercise test, and nuclear and/or CT(A) scan in a specialised cardiology outpatient clinic. The observed sensitivity of current best-in-class diagnostic is 74-84%.

Diagnosis based on the proposed MyCor Classifier methods disclosed herein was superior to the current best-in-class diagnostic.

The methods, compositions, uses, and kit described herein permitted detection with a (simple) blood test. The methods provided a diagnosis (and severity) of ischemic heart disease (angina) with an accuracy >98-99.9% in an all-comers population (non-selected ‘real world’ population). No sub-stratification is needed for cardiovascular risk factors. Blood test according to the methods herein outperformed sophisticated imaging techniques like nuclear imaging and CT (angio) scans. Thus, a first-in-class diagnostic blood test for conditions comprising ischemia and/or neoangiogenesis (such as heart disease) is described in some embodiments herein.

### Example 10—BioBank Clinical Validation

This Example provides further analysis of the first 244 patients. A retrospective analysis of the first ‘real world’ patients with chest pain showed that the procedural failure of the diagnostic test was 1.3% in the last 200 patients. This study provided a proof-of-principle. Based on the analysis of the data, a classifier of 14 biomarkers had an accuracy of 98-99.9%, and a classifier of 2 biomarkers has an accuracy of 82%. Thus, without being limited by theory, a combination of platforms may be indicated. It is further contemplated that validation of quantification of ischemia (based on the methods, kits, compositions, and uses disclosed herein) would allow one to compare treatments and to continue to monitor patients.

### Example 11—Confirmatory Analysis in Ischemic Disease

In the first (“Proposed MyCor Classifier”) analysis of 244 patients (described, for example, in Examples 9 and 10), a proof-of-concept was established (alpha+beta error 0.05-1.7% in non-optimized conditions). No sub-stratification was needed for cardiovascular risk factors.

In a second analysis/study, a refined definition of an ischemic patient population is utilized for the assessed population (n=1000). In the second analysis, adjudication is further optimized, biostatistical analysis is further optimized, sub-stratification explored, optimized and standardized (FDA approved) micro array platform are used, cross-correlation with the extend of ischemia on advanced imaging (including MIBI SPECT and coronary angiography) is analysed, and retrospective and prospective analyses are performed. Based on a consideration of the above factors, a sensitivity of >99% is expected to be obtained in all patients.

Without being limited by theory, preliminary bioinformatics analysis suggest that a classifier can be generated using the blood samples of CAD patients based on in depth bioinformatics (retrospective) analysis of patient cohort by a biostatistician to optimise algorithms, including (but not limited to) Random Forrest plot analysis, (un)-scaled SVM analysis, and Bootstrapping.

Data annotation is useful for diagnosis based on the current practise in cardiology outpatient clinic involving patient interview, ECG, blood work, bicycle test, MSCT, etc., in clinical follow up at 6 months. It is contemplated that clinical follow up can reduce the false negative and false positive allocated patients in the SOC diagnostic group.

In contrast, to diagnosis based on the current practice in cardiology outpatient clinic, advanced imaging (including for example myocardial perfusion imaging with MIBI SPECT) has improved sensitivity and specificity as an advanced myocardial perfusion imaging test. Analysis was performed using Affymetrix Exon 2.0 microarray platform and samples of 244 patients re-analysed using expression profiling.

### Example 12—Product Development Status 3

In an analysis, ischemic RNA and protein signatures are further validated and expanded in a prospective study/registry in a better-defined ischemic patient population (n=5000). Adjudication is further optimized, biostatistical analysis is further optimized, sub-stratification explored, optimized and standardized (FDA approved) micro array platform are used, cross-correlation with the extend of ischemia on advanced imaging (such as MIBI SPECT, and other myocardial perfusion imaging) is analysed, and retrospective and prospective analyses are performed. Diagnostic platform(s) are identified and optimized, for example, microarray (for validation and reference), Luminex assays, Multiplex assay, qPCR, protein assay (ELISA), Nanostring, or direct sequencing.

### Example 13—Isolation of Nucleic Acids

Methods of isolating nucleic acids from cells are well known in the art. For example, methods of isolating nucleic acids from cells using solid phase binding materials without the use of a lysis solution is described in US 2005/0106602 A1, which is hereby incorporated by reference in its entirety. Methods for isolating total RNA from cells are also well known in the art. For example, methods for isolating RNA from cells using an extraction solution comprising formamide is described in U.S. Pat. No. 9,416,357 B2, which is hereby incorporated by reference in its entirety. Several commercially available kits allow for high-throughput analysis-grade nucleic acids to be isolated from cells.

In some embodiments, the method, use, or composition comprises various steps or features that are present as single steps or features (as opposed to multiple steps or features). For example, in one embodiment, the method comprises detecting the condition or a risk thereof in the subject when the expression levels of the specified different gene products are outside of the control ranges, and further includes inhibiting, ameliorating, delaying the onset of, reducing the likelihood of, treating, or preventing a condition comprising perfusion shortage by administering a medical intervention as described herein. The medical intervention may be provided in a single step or administration, or over a period of time, as is suitable in the art. Multiple features or components may be provided in alternate embodiments. In some embodiments, the method, composition, or use comprises one or more medical interventions. In some embodiments, the medical intervention comprises a modulator (such as an inhibitor or enhancer) of a nucleic acid comprising any of nucleotide sequences selected from Tables 1-6, or a peptide encoded by a nucleic acid comprising any of sequences of Tables 1-6.

### Example 14—Clinical Study of Ischemic Biomarker Analysis in Heart Disease Patients

This example covers a clinical study for detecting ischemia in patients without myonecrosis. Currently, patients analyzed for chest pain reminiscent of angina pectoris are analyzed using a combination of including ECG, standard blood works, exercise testing, and CT analysis (including calcium imaging and CT angiography) only to result in an adequate diagnosis in 74% of the cases. Even with advanced perfusion imaging, the adequate/accurate diagnosis of ischemic heart disease does not exceed 84%. In addition, setting up a multi-disciplinary analysis of these complex patients remains a logistical challenge (involving functional testing and imaging, involvement of the dept of cardiology, nuclear medicine, and radiology) and represents a laborious and costly diagnostic track (with disappointing accuracy). In addition, the (extend of) ischemia (or the biological response to ischemia) cannot be readily quantified (or expressed in AU) for use in monitoring of these patients for disease progression or therapy responsiveness.

Subjects selected for the study will be selected from among adults of at least 18 years of age presenting chest pain or other cardiac related symptoms. Excluded subjects will include those who: (1) received percutaneous coronary intervention (PCI) within the last 6 months, (2) coronary artery bypass graft surgery within the past 6 months, (3) are suspected of acute coronary syndrome (acute myocardial infarction and unstable angina) or are Troponin positive, (4) have prior documented myocardial infarction within 30 days prior to cCTA or between cCTA and ICA, (5) have tachycardia or significant arrhythmia, (6) have a known anaphylactic allergy to iodinated contrast, (7) are pregnant or have unknown pregnancy status as well as childbearing potential, (8) have a body mass index compatible with imaging, (9) requires an emergent procedure, (10) displays evidence of ongoing or active clinical instability, including acute chest pain (sudden onset), cardiogenic shock, unstable blood pressure with systolic blood pressure <90 mmHg, and severe congestive heart failure (NYHA III or IV) or acute pulmonary edema, (11) has any active, serious, life-threatening disease with a life expectancy of less than 2 months, (12) is unable to comply with study procedures.

The study will enroll patients with angina suspect complaints and will enroll two cohorts of patients. Cohort 1 (evaluate for sensitivity) will include subjects with angina disease and Cohort 2 of controls (evaluate for specificity) will include subjects without angina disease. The sample size of approximate 180 will provide 90% power to establish that the studied ischemic biomarker profile method is superior over the standard method (Standard Method) at two-sided significance level of 0.05. In the sample size calculation, it is assumed that the sensitivity and specificity are at least 92% for Test Method and no more than 80% for the Control Method.

The study will enroll at least 75 health subjects without angina disease into Cohort 2. In addition, the study will enroll clinical evaluation subjects who present themselves to the participating sites for clinical evaluation for potential angina disease. It is expected that 30% of them will have angina disease (to be included in Cohort 1 analysis) and 70% of them will not have angina disease (to be included in Cohort 2 analysis). The study will enroll at least 600 clinical evaluation subjects to reach at least 180 Cohort 1 subjects.

Subjects who are determined to have the disease by the methods of the present study and Standard Methods may be treated (the investigator can decide to follow them without immediate treatment). All other subjects (subjects who are determined to have angina by only one method and subjects who are determined to no having angina by both methods) will be followed without treatment unless the investigator has determined that it is unethical not to treat the subjects based on well documented clinical observations. If the following treatment occurs within 6 months of chest pain, the subject will be stratified to the treatment group.

A thorough clinical work up which consists of multiple exams and visits to the clinic would be required of the patient, as the next step. An analytical framework presented in FIG. 11, demonstrates the complex decision making involved. For patients, who are suspected of CAD, will undergo invasive coronary angiography furthering their risk of radiation exposure. A test often ordered and completed weeks after initial complaint of chest pain. FIG. 12 provides a thorough summary of sensitivity and specificity results of invasive tests currently used to diagnosis CAD. Study assessments will be performed at the visits and time points outlined in the Time and Events Schedule FIG. 13.

A total of 1000 subjects will be enrolled. Subjects who provide informed consent will provide blood samples at Baseline (10 ml) and prior to imaging (15 ml). The study site will reach out to the subject at month 6 and 12 and capture prognosis, treatment related and if any, hospitalization details.

Any subject who voluntarily withdraws consent or is discontinued from the study prior to completion will be considered as withdrawn from the study. Subjects may be discontinued from the study under any of the following circumstances: (1) occurrence of intolerable AE, as assessed by the investigator or designee; (2) withdrawal of consent; (3) lost to follow-up; (4) Administrative reasons (e.g., sponsor decision); (5) major violation of the protocol; (6) if, in the opinion of the qualified investigator, it is in the best interest of the subject; (7) non-compliance with study requirements and restrictions. Replacement subjects are to be added at the sponsor's discretion, in agreement with the principal investigator.

Subjects will undergo phlebotomy and provide a total of 10 ml blood in an sterile RNA stabilizer tube and 5 ml whole blood at Baseline and prior to cardiac imaging. The investigational site will prepare the samples, as instructed, by the sponsor. The investigational site will be store the RNA collected samples in a secured research area at room temperature prior to shipment to the sponsor. The sponsor will work with each clinical site to determine the appropriate shipment schedule.

Medical histories will be obtained according to the site's SOPs. Medical histories will include demographic data (date of birth, sex, race, and ethnicity); histories of acute, chronic, and infectious disease; surgical and oncologic histories; and any reported conditions affecting major body systems. All findings on medical history will be evaluated by the investigator for clinical significance. All medications (prescription and non-prescription, herbal medications/natural health products, and investigational drugs) taken by the subjects during the 30 days prior to consent will be recorded in the source documentation as medication history. The physical examination, assessing the subject's overall health and physical condition, will be performed according to the site's SOPs. Height, weight, and BMI will be recorded. Vital signs will be measured and managed according to the site's SOPs. Vital signs will consist of systolic and diastolic blood pressure (mmHg), heart rate (bpm), respiratory rate (breaths/min), and oral temperature (° C.). Safety 12-lead ECGs will be performed and interpreted according to the site's SOPs or if complete Telemetry is available, data is acceptable. If the following treatment occurs within 6 months of chest pain, the subject will be stratified to treatment group: (1) treatment with beta blockers, antiangina Medications, antihypertensive drug, calcium channel blocker and anticoagulant; (2) angioplasty, coronary stent, cardiac catherization, revascularization, coronary angioplasty; (3) coronary artery bypass surgery and hybrid coronary revascularization.

All analyses will be performed using SAS version 9.3 or higher, run on the Microsoft Windows Server 2003 R2 operating system.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

For each method of described herein, relevant compositions for use in the method are expressly contemplated, uses of compositions in the method, and, as applicable, methods of making a medicament for use in the method are also expressly contemplated. For example, for methods of inhibiting, ameliorating, delaying the onset of, reducing the likelihood of, treating, or preventing a condition comprising perfusion shortage (or neoangiogenesis), corresponding compositions for use in the corresponding method are also contemplated, as are uses of the corresponding composition to inhibit, ameliorate, delaying the onset of, reduce the likelihood of, treat, or prevent a condition comprising perfusion shortage. In some cases, disclosed nucleotide sequences as medical compounds or targets may be used to promote ischemic conditions, such as in tumor angiogenesis.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. For example, “about 5”, shall include the number 5. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

Lengthy table referenced here
US20230416822A1-20231228-T00001
Please refer to the end of the specification for access instructions.

Lengthy table referenced here
US20230416822A1-20231228-T00002
Please refer to the end of the specification for access instructions.

Lengthy table referenced here
US20230416822A1-20231228-T00003
Please refer to the end of the specification for access instructions.

Lengthy table referenced here
US20230416822A1-20231228-T00004
Please refer to the end of the specification for access instructions.

Lengthy table referenced here
US20230416822A1-20231228-T00005
Please refer to the end of the specification for access instructions.

Lengthy table referenced here
US20230416822A1-20231228-T00006
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20230416822A1). An electronic copy of the table will also be available from the USPTO upon request and payment of the fee set forth in 37 CFR 1.19(b)(3).

1. A method for analyzing a biological sample, comprising:
(a) receiving the biological sample from a subject;
(b) wherein when the subject is a female subject, assaying expression levels of two or more genes from a female targeted set of genes to generate an output comprising the expression levels from (b) indicative of the female subject's having a condition selected from ischemic cardiovascular disease, ischemic heart disease, coronary artery disease, cardiovascular congenital disease, hypertrophic or dilating cardiomyopathy, heart failure;
(c) wherein when the subject is a male subject, assaying expression levels of two or more genes from a male targeted set of genes to generate an output comprising the expression levels from (c) indicative of the male subject's having a condition selected from ischemic cardiovascular disease, ischemic heart disease, coronary artery disease, cardiovascular congenital disease, hypertrophic or dilating cardiomyopathy, heart failure; and
(d) providing a treatment to the subject, the treatment selected from the group consisting of beta-blockers, calcium channels antagonists, nitrates, aspirin, cholesterol-lowering compounds, angiotensin converting enzyme (ACE) inhibitors, and ranolazine.

2-4. (canceled)

5. The method of claim 1, wherein the female target set of genes comprises one or more, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, or three hundred or more of the genes selected from the group consisting of ZNF807, LOC100130331, LOC100130872, LINC01502, FAM66A, SRGAP3-AS3, MIR1185-1, MIR1266, MIR1261, MIR4262, MTRNR2L10, MIR3928, LOC100507065, LOC100507642, MIR1273F, MIR4519, MIR4799, MIR4747, PTPRU, LOC100996660, DNAL4, XXYLT1-AS2, LOC101927230, LOC101927377, LOC101927479, LOC101927503, LINC01186, LOC101927735, LOC101927880, LINC01486, LOC101928775, RHOXF1P1, LOC101929108, LOC101929330, LOC101929696, CEACAM5, LAMTOR5, CELP, SLC27A4, RCC1, STMN2, CAPN11, FDX1L, MIR155HG, CSMD2, PNMA5, HSPA12B, CLN5, LRRC38, COL3A1, ARHGEF19, PROKR2, COX6C, PTRH1, KRTAP13-1, ASB9, CRYGC, VTI1A, BEST3, SLC22A31, ADIG, LINC01104, PYDC2, PRSS3P2, OTUD7A, DCC, RBM46, TCEANC, ADAMTS17, EFNA5, CELSR2, U2AF1L4, PIKFYVE, KCTD6, PTPLB, EMX1, OR9I1, JAZF1, LAMB4, FOXC2, NCOA6, MESDC2, FBX028, DNMBP, U2SURP, ABCA6, CBX5, CBLC, KIF4A, C20orfI 66-AS1, NUDT8, NUP210P1, SMCOI, MAP7D2, ALX3, FAM133B, SUN2, HECTD1, WIPI2, SNORD53, GJA8, DHDH, GPR4, MRGPRG-AS 1, LOC283731, LINC00304, ZNF763, IGHV1-58, LOC284933, RBMY1A3P, GPX5, MED4, C6orfI5, GUCY1A2, GUCA2A, HUNK, TFAP2E, POTEA, LEUTX, OR2T3, IGFALS, C19orf45, FAM99A, IGFL3, OR2M1P, FAM90A25P, C8orf86, TMEM8C, PABPN1L, LIG4, FLJ26245, KRT16P2, PRSS57, LTBP3, MIR215, ARSF, MEF2C, IQSEC3, LOC441179, ZNRF2P1, MT1M, MTIF2, MY07A, NDUFB9, ATP2B1, MIR422A, CEND1, LARS, UFM1, PDHA2, CES1P1, PLTP, LURAP1, MRPS21, MED1, PPP1R3C, RBM41, ASIC4, ZNF821, PPP1R9A, TRMU, UTP6, CCDC177, TBC1D24, GPR158, NYNRIN, BAI2, GBA3, PTPRO, RAC3, CDH26, RPLPO, BGN, FAM204A, PJA1, MT1IP, SHBG, LOC646513, SLA, SKOR2, SLC34A1, SNRPD1, SOX9, SP1, MIR532, TDG, TNNI3, DNLZ, LOC729739, CBA, VIPR2, USP7, DDX39B, ICE2, DCSTAMP, HIST1H4J, ZNF484, REG4, LRRIQ1, FAM126A, DOCK7, RNMT, ST3GAL5, GAL3ST3, SLC25A21, FATE1, UNC5A, TBX18, FIBP, DLGAP1, MGME1, SYCE1, HAND1, TNFRSF8, MAP4K4, GOSR1, SETDB1, SLC23A2, DOPEY2, and TBX5-AS1.

6-7. (canceled)

8. The method of claim 1, wherein the male target set of genes comprises one or more, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, or three hundred or more of the genes selected from the group consisting of SNORD115-37, SNORD115-24, SNORA11B, MIR876, COL4A2-AS2, LOC100130373, LOC100130548, LOC100131496, LINC00240, BCL2L11, MED 16, ERVV-2, KRTAP21-3, LOC100289230, LOC100289283, MIR1275, MIR1912, MIR1302-7, MIR1231, MIR1238, MIR1179, MIR1908, MIR1237, MIR2116, CSN1S2BP, MIR3148, MIR4280, MIR3116-1, MIR3129, MIR4275, MIR4326, MIR4254, LINC00673, MIR3926-2, MIR3678, MIR548Z, MIR3657, MIR3935, LOC100505478, LINC01364, C16orf95, LINC00837, MIR3529, MIR4475, MIR4492, MIR4784, MIR4732, MIR4684, MIR4773-2, MIR4429, MIR4442, LINC00507, LOC100996345, CYYR1-AS1, LOC100996635, C D K4, LOC101927053, PDZRN3-AS1, MEOX2-AS1, LINCOI 186, NA, GBAT2, LINC01214, LOC101928101, LOC101928118, NA, LOC101928195, LOC101928385, TCONS_00029157, LOC101928668, LINC01450, NA, LOC101928823, LOC101928919, LINC01262, LOC101929031, LOC101929159, LOC101929468, LOC101929497, ZNF267, CHERP, IFI44, FUT9, RFPL2, IFI44L, MAPRE2, SLC27A5, CHD1, RABL2B, FSTL1, MRPL3, GLB1L3, GPR182, CHRM5, SMIM12, LRRC37B, SMYD4, TBCB, KLHDC3, RPL39L, CLTB, PRSS30P, PCP2, FITM2, SNTN, TMEM68, OTUD6A, CRP, DYNLL2, CRYGD, NA, TMC8, CSTB, SLC35F3, CCDC140, WDSUB1, SH3D19, TEX28, FEZF1-AS1, ESC02, ZNF645, CYP51A1, DAB1, ZCCHC12, DNMT3A, DRP2, EGR2, ABCA2, CDY2B, SENP5, EVI2B, ALCAM, ABCD1, F12, FAH, OR5A2, CCDC89, FABP5P3, FGF4, FGF14, DIP2A, ATP1 IB, NUP210, EXOC6B, ARC, SASH1, GRIP1, SEC61G, SCRIB, POLA2, GABARAPL1, DEFB108B, DEFB113, FTH1P3, CDRT15L2, HEATR9, ALS2CL, ASPM, OR2F1, GATA2, PLA2G2D, GCNT1, AGOI, OR7A5, OR1J2, SNORA70, GNAI3, BCL9L, OTOGL, SLC46A3, EWSAT1, LINC00927, C16orf54, MAMSTR, MCHR1, LOC284825, IGHD2-21, IGHD2-15, EOGT, C8orf31, IGLV10-54, IGLV3-12, SNX24, ARHGAP35, SCG3, PYCR2, SLC39A3, H3F3B, ANXA5, CXXC1, CDR2L, HNF4A, HNRNPK, SNORD56B, KRTAP20-1, KRTAP20-2, LOC339166, ZFP69, LOC340074, KIAA1875, OR2T8, PAQR9, IFNGR1, IGFBP1, KRTAPIO-IO, IL13RA1, KCNA3, KCNA5, TPTE2P6, LHFPL3, KCNJ10, LINC00162, KCNS3, KRTAP5-1, CC2D2B, RPRML, LRRC14B, OR52N2, LAMA4, LIPE, LINC00200, LINC00668, ZNF880, LOC400756, ASB18, LRRDI, LDLRAD2, LYN, MIR127, MIR210, MIR181A1, MIR9-1, MIR98, MIR99B, MIR17HG, MAFG, MAS1, MAX, MLF1, SUGT1P1, RPS26P11, MYOIO, NDUFB9, NKX3-1, NOS1, NRGN, MIR375, DUOX2, RRP15, NMD3, KLHL5, RPL26L1, EGFL7, PCYT1A, CDC40, UFC1, ERGIC3, SF3B6, PFDN4, PIK3C2G, DDIT4, EGLN1, FAM20A, HEATR3, CEP72, THSD1, PSME2, MIR362, MIR494, MIR520C, MIR51662, MIR502, MIR508, ERMN, RANBP10, SH3RF1, FBRSL1, RET, EXOC4, TGIF2, RPL3L, BCORL1, CCL5, SDCBP, RRAGC, XYLT2, ACOT6, POPDC3, LINC00535, CCT6P1, TSG1, NA, BLVRA, NARFL, CSMD1, SGSH, NDST4, NA, EFTUD1P1, SIPA1, SLC5A4, PCP4L1, SLC9A2, LYNX1, MIR539, CAPN15, SPAG1, SPRR3, SQLE, SRI, SCARNA2, SCARNA16, TACR1, SNORD66, SNORD84, SNORD91A, SNORD97, MIR552, MIR563, MIR591, TCF19, TNP1, MIR660, SDHAP3, LOC729224, FAHD2CP, SEC14L6, MEIS1-AS3, WNT9A, SNORD113-3, SNORD114-3, SNORD1 14-9, SNORD114-18, SNORD114-27, SNORD114-30, MIR758, MIR668, ZNF134, PPDPF, OR5H2, CERS4, OGFOD2, LIN28A, NEK11, DCAKD, GPR157, EFHC2, CXXC4, PBX4, CPTP, NPRL3, OR2B2, AKAP17A, PCDH11Y, FAM103A1, TRAPPC9, HYAL3, CASP5, TTTY11, RAB34, NACAP1, SPOP, ZMYND12, LLPH, C22orf23, GTPBP3, SUV420H2, HAT1, SHANK3, KIAA1755, DENR, NPFF, MARCO, JRKL, PAGE1, PABPC4, SYNGAP1, SGPL1, BUD31, HIST1H3F, SYT8, MAP7, ZFAND2A, RGN, VAPB, RCSD1, SLIT2, NCR2, PAGE4, RNF7, MTFR1, CD59, and ATP2C2.

9. The method of claim 1, further comprising obtaining a range of control expression levels of the female targeted set of genes and comparing the expression levels of (b) with the range of control expression levels of the female targeted set of genes.

10. (canceled)

11. The method of claim 1, further comprising obtaining a range of control expression levels of the male targeted set of genes and comparing the expression levels of (c) with the range of control expression levels of the male targeted set of genes.

12-13. (canceled)

14. The method of claim 1, wherein the biological sample comprises isolated hematopoietic endothelial precursor cells (EPCs), fractions thereof, or secretions thereof.

15-18. (canceled)

19. A method for analyzing a biological sample obtained from a subject, comprising:
(i) subjecting a plurality of hematopoietic endothelial precursor cells (EPCs), fractions of the plurality of hematopoietic EPCs, secretions of the plurality of hematopoietic EPCs, or any combination thereof isolated from the biological sample, to a gene expression analysis, wherein the gene expression analysis comprises assaying expression levels of two or more genes from a plurality of identified genes;
(ii) generating an output comprising the gene expression levels, wherein the gene expression levels are indicative of the subject having a condition selected from ischemic cardiovascular disease, ischemic heart disease, coronary artery disease, cardiovascular congenital disease, hypertrophic or dilating cardiomyopathy, heart failure; and
(iii) providing a treatment to the subject, the treatment selected from the group consisting of beta-blockers, calcium channels antagonists, nitrates, aspirin, cholesterol-lowering compounds, angiotensin converting enzyme (ACE) inhibitors, and ranolazine.

20. The method of claim 19, wherein the plurality of hematopoietic EPCs comprises a cell surface marker of Flk-I/KDR.

21-24. (canceled)

25. The method of claim 19, wherein the plurality of identified genes comprises a female targeted set of genes and a male targeted set of genes, wherein the female targeted set of genes comprises a plurality of genes listed in Tables 1, 3, and 5, wherein the male targeted set of genes comprises a plurality of genes listed in Tables 2, 4, and 6.

26-27. (canceled)

28. The method of claim 19, further comprising obtaining a range of control expression levels of a female targeted set of genes or of a male targeted set of genes and comparing the gene expression levels from (i) to a range of control expression levels.

29-67. (canceled)

68. The method of claim 1, wherein when the subject is a female subject, further comprising assaying less than 60 genes from the plurality of genes listed in Tables 1, 3, and 5.

69. The method of claim 1, wherein when the subject is a male subject, further comprising assaying less than 40 genes from the plurality of genes listed in Tables 2, 4, and 6.

70. The method of claim 1, wherein the sample comprises hematopoietic EPCs, and wherein the hematopoietic EPCs are positive for makers selected from the group consisting of VEGFR2, CD309, Flt1, Flt4, KDRT, VEGFRT, CXCR4, CD31, CD34, CD105, CD133, Sca-1, and c-Kit.

71. The method of claim 1, wherein the sample comprises isolated Flk1+ cells, fractions thereof, or secretions thereof.

72. The method of claim 19, wherein the female target set of genes comprises one or more, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, or three hundred or more of the genes selected from the group consisting of ZNF807, LOC100130331, LOC100130872, LINC01502, FAM66A, SRGAP3-AS3, MIR1185-1, MIR1266, MIR1261, MIR4262, MTRNR2L10, MIR3928, LOC100507065, LOC100507642, MIR1273F, MIR4519, MIR4799, MIR4747, PTPRU, LOC100996660, DNAL4, XXYLT1-AS2, LOC101927230, LOC101927377, LOC101927479, LOC101927503, LINC01186, LOC101927735, LOC101927880, LINC01486, LOC101928775, RHOXF1P1, LOC101929108, LOC101929330, LOC101929696, CEACAM5, LAMTOR5, CELP, SLC27A4, RCC1, STMN2, CAPN11, FDX1L, MIR155HG, CSMD2, PNMA5, HSPA12B, CLN5, LRRC38, COL3A1, ARHGEF19, PROKR2, COX6C, PTRH1, KRTAP13-1, ASB9, CRYGC, VTI1A, BEST3, SLC22A31, ADIG, LINC01104, PYDC2, PRSS3P2, OTUD7A, DCC, RBM46, TCEANC, ADAMTS17, EFNA5, CELSR2, U2AF1L4, PIKFYVE, KCTD6, PTPLB, EMX1, OR9I1, JAZF1, LAMB4, FOXC2, NCOA6, MESDC2, FBX028, DNMBP, U2SURP, ABCA6, CBX5, CBLC, KIF4A, C20orfI 66-AS1, NUDT8, NUP210P1, SMCOI, MAP7D2, ALX3, FAM133B, SUN2, HECTD1, WIPI2, SNORD53, GJA8, DHDH, GPR4, MRGPRG-AS 1, LOC283731, LINC00304, ZNF763, IGHV1-58, LOC284933, RBMY1A3P, GPX5, MED4, C6orfI5, GUCY1A2, GUCA2A, HUNK, TFAP2E, POTEA, LEUTX, OR2T3, IGFALS, C19orf45, FAM99A, IGFL3, OR2M1P, FAM90A25P, C8orf86, TMEM8C, PABPN1L, LIG4, FLJ26245, KRT16P2, PRSS57, LTBP3, MIR215, ARSF, MEF2C, IQSEC3, LOC441179, ZNRF2P1, MT1M, MTIF2, MY07A, NDUFB9, ATP2B1, MIR422A, CEND1, LARS, UFM1, PDHA2, CES1P1, PLTP, LURAP1, MRPS21, MED1, PPP1R3C, RBM41, ASIC4, ZNF821, PPP1R9A, TRMU, UTP6, CCDC177, TBC1D24, GPR158, NYNRIN, BAI2, GBA3, PTPRO, RAC3, CDH26, RPLPO, BGN, FAM204A, PJA1, MT1IP, SHBG, LOC646513, SLA, SKOR2, SLC34A1, SNRPD1, SOX9, SP1, MIR532, TDG, TNNI3, DNLZ, LOC729739, CBA, VIPR2, USP7, DDX39B, ICE2, DCSTAMP, HIST1H4J, ZNF484, REG4, LRRIQ1, FAM126A, DOCK7, RNMT, ST3GAL5, GAL3ST3, SLC25A21, FATE1, UNC5A, TBX18, FIBP, DLGAP1, MGME1, SYCE1, HAND1, TNFRSF8, MAP4K4, GOSR1, SETDB1, SLC23A2, DOPEY2, and TBX5-AS1.

73. The method of claim 19, wherein the male target set of genes comprises one or more, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, or three hundred or more of the genes selected from the group consisting of SNORD115-37, SNORD115-24, SNORA11B, MIR876, COL4A2-AS2, LOC100130373, LOC100130548, LOC100131496, LINC00240, BCL2L11, MED 16, ERVV-2, KRTAP21-3, LOC100289230, LOC100289283, MIR1275, MIR1912, MIR1302-7, MIR1231, MIR1238, MIR1179, MIR1908, MIR1237, MIR2116, CSN1S2BP, MIR3148, MIR4280, MIR3116-1, MIR3129, MIR4275, MIR4326, MIR4254, LINC00673, MIR3926-2, MIR3678, MIR548Z, MIR3657, MIR3935, LOC100505478, LINC01364, C16orf95, LINC00837, MIR3529, MIR4475, MIR4492, MIR4784, MIR4732, MIR4684, MIR4773-2, MIR4429, MIR4442, LINC00507, LOC100996345, CYYR1-AS1, LOC100996635, CDK4, LOC101927053, PDZRN3-AS1, MEOX2-AS1, LINCOI 186, NA, GBAT2, LINC01214, LOC101928101, LOC101928118, NA, LOC101928195, LOC101928385, TCONS_00029157, LOC101928668, LINC01450, NA, LOC101928823, LOC101928919, LINC01262, LOC101929031, LOC101929159, LOC101929468, LOC101929497, ZNF267, CHERP, IFI44, FUT9, RFPL2, IFI44L, MAPRE2, SLC27A5, CHD1, RABL2B, FSTL1, MRPL3, GLB1L3, GPR182, CHRM5, SMIM12, LRRC37B, SMYD4, TBCB, KLHDC3, RPL39L, CLTB, PRSS30P, PCP2, FITM2, SNTN, TMEM68, OTUD6A, CRP, DYNLL2, CRYGD, NA, TMC8, CSTB, SLC35F3, CCDC140, WDSUB1, SH3D19, TEX28, FEZF1-AS1, ESC02, ZNF645, CYP51A1, DAB1, ZCCHC12, DNMT3A, DRP2, EGR2, ABCA2, CDY2B, SENP5, EVI2B, ALCAM, ABCD1, F12, FAH, OR5A2, CCDC89, FABP5P3, FGF4, FGF14, DIP2A, ATP1 IB, NUP210, EXOC6B, ARC, SASH1, GRIP1, SEC61G, SCRIB, POLA2, GABARAPL1, DEFB108B, DEFB113, FTH1P3, CDRT15L2, HEATR9, ALS2CL, ASPM, OR2F1, GATA2, PLA2G2D, GCNT1, AGOI, OR7A5, OR1J2, SNORA70, GNAI3, BCL9L, OTOGL, SLC46A3, EWSAT1, LINC00927, C16orf54, MAMSTR, MCHR1, LOC284825, IGHD2-21, IGHD2-15, EOGT, C8orf31, IGLV10-54, IGLV3-12, SNX24, ARHGAP35, SCG3, PYCR2, SLC39A3, H3F3B, ANXA5, CXXC1, CDR2L, HNF4A, HNRNPK, SNORD56B, KRTAP20-1, KRTAP20-2, LOC339166, ZFP69, LOC340074, KIAA1875, OR2T8, PAQR9, IFNGR1, IGFBP1, KRTAPIO-IO, IL13RA1, KCNA3, KCNA5, TPTE2P6, LHFPL3, KCNJ10, LINC00162, KCNS3, KRTAP5-1, CC2D2B, RPRML, LRRC14B, OR52N2, LAMA4, LIPE, LINC00200, LINC00668, ZNF880, LOC400756, ASB18, LRRDI, LDLRAD2, LYN, MIR127, MIR210, MIR181A1, MIR9-1, MIR98, MIR99B, MIR17HG, MAFG, MAS1, MAX, MLF1, SUGT1P1, RPS26P11, MYOIO, NDUFB9, NKX3-1, NOS1, NRGN, MIR375, DUOX2, RRP15, NMD3, KLHL5, RPL26L1, EGFL7, PCYT1A, CDC40, UFC1, ERGIC3, SF3B6, PFDN4, PIK3C2G, DDIT4, EGLN1, FAM20A, HEATR3, CEP72, THSD1, PSME2, MIR362, MIR494, MIR520C, MIR51662, MIR502, MIR508, ERMN, RANBP10, SH3RF1, FBRSL1, RET, EXOC4, TGIF2, RPL3L, BCORL1, CCL5, SDCBP, RRAGC, XYLT2, ACOT6, POPDC3, LINC00535, CCT6P1, TSG1, NA, BLVRA, NARFL, CSMD1, SGSH, NDST4, NA, EFTUD1P1, SIPA1, SLC5A4, PCP4L1, SLC9A2, LYNX1, MIR539, CAPN15, SPAG1, SPRR3, SQLE, SRI, SCARNA2, SCARNA16, TACR1, SNORD66, SNORD84, SNORD91A, SNORD97, MIR552, MIR563, MIR591, TCF19, TNP1, MIR660, SDHAP3, LOC729224, FAHD2CP, SEC14L6, MEIS1-AS3, WNT9A, SNORD113-3, SNORD114-3, SNORD1 14-9, SNORD114-18, SNORD114-27, SNORD114-30, MIR758, MIR668, ZNF134, PPDPF, OR5H2, CERS4, OGFOD2, LIN28A, NEK11, DCAKD, GPR157, EFHC2, CXXC4, PBX4, CPTP, NPRL3, OR2B2, AKAP17A, PCDH11Y, FAM103A1, TRAPPC9, HYAL3, CASP5, TTTY11, RAB34, NACAP1, SPOP, ZMYND12, LLPH, C22orf23, GTPBP3, SUV420H2, HAT1, SHANK3, KIAA1755, DENR, NPFF, MARCO, JRKL, PAGE1, PABPC4, SYNGAP1, SGPL1, BUD31, HIST1H3F, SYT8, MAP7, ZFAND2A, RGN, VAPB, RCSD1, SLIT2, NCR2, PAGE4, RNF7, MTFR1, CD59, and ATP2C2.

74. The method of claim 19, wherein when the subject is a female subject, further comprising assaying less than 60 genes from the plurality of genes listed in Tables 1, 3, and 5.

75. The method of claim 19, wherein when the subject is a male subject, further comprising assaying less than 40 genes from the plurality of genes listed in Tables 2, 4, and 6.

76. The method of claim 19, wherein the sample comprises hematopoietic EPCs, and wherein the hematopoietic EPCs are positive for makers selected from the group consisting of VEGFR2, CD309, Flt1, Flt4, KDRT, VEGFRT, CXCR4, CD31, CD34, CD105, CD133, Sca-1, and c-Kit.

78. The method of claim 19, wherein the sample comprises isolated Flk1+ cells, fractions thereof, or secretions thereof.
